SEQUENCE LISTING (30 sequences)

SEQ ID NO: 1 - DNA, 365 bp, Artificial Sequence
  source: /note="Description of Artificial Sequence: Synthetic polynucleotide"
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctca

SEQ ID NO: 2 - protein, 121 aa, Artificial Sequence
  source: /note="Description of Artificial Sequence: Synthetic polypeptide"
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 3 - DNA, 24 bp, Artificial Sequence
  source: /note="Description of Artificial Sequence: Synthetic oligonucleotide"
ggattcacctttagcagatctgcc

SEQ ID NO: 4 - protein, 8 aa, Artificial Sequence
  source: /note="Description of Artificial Sequence: Synthetic peptide"
GFTFSRSA

SEQ ID NO: 5 - DNA, 24 bp, Artificial Sequence
  source: /note="Description of Artificial Sequence: Synthetic oligonucleotide"
attagtggtagtggtggtcgaaca

SEQ ID NO: 6 - protein, 8 aa, Artificial Sequence
  source: /note="Description of Artificial Sequence: Synthetic peptide"
ISGSGGRT

SEQ ID NO: 7 - DNA, 45 bp, Artificial Sequence
  source: /note="Description of Artificial Sequence: Synthetic oligonucleotide"
gcgaaagattcgtatactaccagttggtacggaggtatggacgtc

SEQ ID NO: 8 - protein, 15 aa, Artificial Sequence
  source: /note="Description of Artificial Sequence: Synthetic peptide"
AKDSYTTSWYGGMDV

SEQ ID NO: 9 - DNA, 323 bp, Artificial Sequence
  source: /note="Description of Artificial Sequence: Synthetic polynucleotide"
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacga

SEQ ID NO: 10 - protein, 107 aa, Artificial Sequence
  source: /note="Description of Artificial Sequence: Synthetic polypeptide"
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKR

SEQ ID NO: 11 - DNA, 18 bp, Artificial Sequence
  source: /note="Description of Artificial Sequence: Synthetic oligonucleotide"
cagggtattttcagctgg

SEQ ID NO: 12 - protein, 6 aa, Artificial Sequence
  source: /note="Description of Artificial Sequence: Synthetic peptide"
QGIFSW

SEQ ID NO: 13 - DNA, 9 bp, Artificial Sequence
  source: /note="Description of Artificial Sequence: Synthetic oligonucleotide"
gctgcttcc

SEQ ID NO: 14 - protein, 3 aa, Artificial Sequence
  source: /note="Description of Artificial Sequence: Synthetic peptide"
AAS

SEQ ID NO: 15 - DNA, 27 bp, Artificial Sequence
  source: /note="Description of Artificial Sequence: Synthetic oligonucleotide"
caacaggctaacagtgtcccgatcacc

SEQ ID NO: 16 - protein, 9 aa, Artificial Sequence
  source: /note="Description of Artificial Sequence: Synthetic peptide"
QQANSVPIT

SEQ ID NO: 17 - DNA, 1349 bp, Artificial Sequence
  source: /note="Description of Artificial Sequence: Synthetic polynucleotide"
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 448 aa, Artificial Sequence
  source: /note="Description of Artificial Sequence: Synthetic polypeptide"
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 644 bp, Artificial Sequence
  source: /note="Description of Artificial Sequence: Synthetic polynucleotide"
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 213 aa, Artificial Sequence
  source: /note="Description of Artificial Sequence: Synthetic polypeptide"
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, Artificial Sequence
  source: /note="Description of Artificial Sequence: Synthetic peptide"
GFTFRDYA

SEQ ID NO: 22 - protein, 8 aa, Artificial Sequence
  source: /note="Description of Artificial Sequence: Synthetic peptide"
ISGSGGNT

SEQ ID NO: 23 - protein, 16 aa, Artificial Sequence
  source: /note="Description of Artificial Sequence: Synthetic peptide"
AKDRLSITIRPRYYGL

SEQ ID NO: 24 - protein, 11 aa, Artificial Sequence
  source: /note="Description of Artificial Sequence: Synthetic peptide"
QSLLYSIGYNY

SEQ ID NO: 25 - protein, 3 aa, Artificial Sequence
  source: /note="Description of Artificial Sequence: Synthetic peptide"
LGS

SEQ ID NO: 26 - protein, 9 aa, Artificial Sequence
  source: /note="Description of Artificial Sequence: Synthetic peptide"
MQALQTPYT

SEQ ID NO: 27 - protein, 124 aa, Artificial Sequence
  source: /note="Description of Artificial Sequence: Synthetic polypeptide"
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 28 - protein, 112 aa, Artificial Sequence [sequence omitted]

SEQ ID NO: 29 - protein, 451 aa, Artificial Sequence
  source: /note="Description of Artificial Sequence: Synthetic polypeptide"
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 219 aa, Artificial Sequence
  source: /note="Description of Artificial Sequence: Synthetic polypeptide"
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0340101 A1
(43) Pub. Date: Oct. 26, 2023

(54) METHODS FOR TREATING OR PREVENTING ALLERGIC ASTHMA BY ADMINISTERING AN IL-33 ANTAGONIST AND/OR AN IL-4R ANTAGONIST

(71) Applicants: SANOFI BIOTECHNOLOGY, Paris (FR); REGENERON PHARMACEUTICALS, INC., Tarrytown, NY (US)

(72) Inventors: Helene GOULAOUIC, Paris (FR); El-Bdaoui HADDAD, Bridgewater, NJ (US); Sara HAMON, Tarrytown, NY (US); Sivan HAREL, Tarrytown, NY (US); Georgios KALLIOLIAS, Tarrytown, NY (US); Marcella RUDDY, Tarrytown, NY (US)

(21) Appl. No.: 17/786,226

(22) PCT Filed: Dec. 22, 2020
(86) PCT No.: PCT/US2020/066559

§ 371 Date: Jun. 16, 2022

Related U.S. Application Data
(60) Provisional application No. 62/964,970, filed on Jan. 23, 2020.
(60) Provisional application No. 62/952,996, filed on Dec. 23, 2019.

(51) Int. Cl. A61P 37/08; C07K 16/24; A61P 11/06; C07K 16/28

(52) U.S. Cl. CPC C07K 16/244; A61P 11/06; A61P 37/08; C07K 16/2866; A61K 39/00

(57) ABSTRACT

Methods for treating or preventing allergic asthma and associated conditions in a subject are provided. Certain methods disclosed here comprise administering to a subject in need thereof a therapeutic composition comprising an interleukin-33 (IL-33) antagonist, such as an anti-IL-33 antibody. Other methods disclosed here comprise administering to a subject in need thereof a therapeutic composition comprising an interleukin-4R (IL-4R) antagonist, such as an anti-IL-4R antibody. Still other methods disclosed here comprise administering to a subject in need thereof a first therapeutic composition comprising an interleukin-33 (IL-33) antagonist, such as an anti-IL-33 antibody, and a second therapeutic composition comprising an interleukin-4 receptor (IL-4R) antagonist, such as an anti-IL-4R antibody. Allergic asthma-associated signature genes are provided. Methods of altering (e.g., decreasing) an expression level of one or more allergic asthma-associated signature genes in a subject having allergic asthma are provided.

### RELATED APPLICATIONS

This application is a 35 U.S.C. §371 filing of International Patent Application No. PCT/US2020/066559, filed Dec. 22, 2020, which claims priority to U.S. Provisional Pat. Application Serial Nos. 62/952,996, filed Dec. 23, 2019, and 62/964,970, filed Jan. 23, 2020. The entire disclosure of each of these applications is hereby incorporated herein by reference in its entirety.

### SEQUENCE LISTING

[0001.1] The instant application contains a Sequence Listing which has been submitted electronically in ASCII format and is hereby incorporated by reference in its entirety. Said ASCII copy, created on Jun. 16, 2022, is named 729332_SA9-284US_ST25.txt and is 25,972 bytes in size.

### FIELD OF THE INVENTION

The invention relates to the treatment and/or prevention of allergic asthma and related conditions. More specifically, the invention relates to the administration of an interleukin-33 (IL-33) antagonist to treat or prevent allergic asthma in a patient in need thereof. The invention also relates to the administration of an interleukin-4 (IL-4R) antagonist to treat or prevent allergic asthma in a patient in need thereof. Finally, the invention relates to the administration of an IL-33 (IL-33) antagonist and an interleukin-4 receptor (IL-4R) antagonist to treat or prevent allergic asthma in a patient in need thereof.

### BACKGROUND

Asthma is a chronic inflammatory disease of the airways characterized by airway hyper-responsiveness, acute and chronic bronchoconstriction, airway edema, and mucus plugging. The inflammation component of asthma is thought to involve many cell types, including mast cells, eosinophils, T lymphocytes, neutrophils, and epithelial cells, and their biological products. Patients with asthma most often present with symptoms of wheezing, shortness of breath, cough, and chest tightness. For most asthma patients, a regimen of controller therapy and bronchodilator therapy are used to provide long-term control. Inhaled corticosteroids (ICS) are considered the “gold standard” in controlling asthma symptoms, and inhaled beta2-agonists are the most effective bronchodilators currently available.

Type 2-high asthma is the most prevalent type of persistent asthma (Fahy (2015) Nat. Rev. Immunol. 15:57-65). It includes the overlapping phenotypes allergic asthma (characterized by increased expression of specific immunoglobulin E (IgE) to aeroallergens) and eosinophilic asthma (characterized by blood and/or airway/tissue eosinophilia) (Fahy, Supra; Campo et al. (2013) J. Investig. Allergol. Clin. Immunol. 23:76-88; Wenzel (2012) Clin Exp Allergy 42:650-8).

Allergic asthma is the most common type of asthma. Allergic sensitization is a strong risk factor for asthma inception and severity in children and in adults (Gough et al. (2015) Pediatr. Allergy Immunol. 26:431-437). Current allergic asthma therapies that address symptoms and the ongoing inflammatory process of the disease do not affect the underlying, dysregulated immune response and, therefore, are very limited in controlling allergic asthma progression (Dhami et al. (2017) Eur. J. Allergy Clin. Immunol. 72(12):1825-1848)).

A need exists in the art for novel targeted therapies for the treatment and/or prevention of asthma, e.g., allergic asthma.

### BRIEF SUMMARY OF THE INVENTION

According to one aspect, a method for treating allergic asthma in a subject in need thereof comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided for use to treat allergic asthma in a subject in need thereof.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. According to certain exemplary embodiments, the antibody or antigen-binding fragment thereof comprises REGN3500.

In certain exemplary embodiments, the antibody or antigen-binding fragment thereof is administered intravenously at a dose of 10 mg/kg. In certain exemplary embodiments, the antibody or antigen binding fragment thereof is administered subcutaneously at a dose of about 0.1 mg to about 600 mg, about 100 mg to about 400 mg, or about 300 mg. In certain exemplary embodiments, the antibody or antigen binding fragment thereof is administered subcutaneously at an initial dose of about 600 mg or about 300 mg. In certain exemplary embodiments, the antibody or antigen binding fragment thereof is administered subcutaneously in one or more secondary doses of about 300 mg.

According to another aspect, a method for treating allergic asthma in a subject in need thereof comprising administering to the subject an antibody or antigen-binding fragment thereof that specifically binds interleukin-4R (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4R (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to treat allergic asthma in a subject in need thereof.

In certain exemplary embodiments, the antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the antibody or antigen binding fragment thereof is administered at a dose of about 0.1 mg to about 600 mg, about 100 mg to about 400 mg, or about 300 mg. In certain exemplary embodiments, the antibody or antigen binding fragment thereof is administered at an initial dose of about 600 mg. In certain exemplary embodiments, the antibody or antigen binding fragment thereof is administered in one or more secondary doses of about 300 mg.

In certain exemplary embodiments, the antibody or antigen-binding fragment thereof is administered every week (q1w), every other week (q2w), every three weeks (q3w), or every four weeks (q4w). In certain exemplary embodiments, the antibody or antigen-binding fragment thereof is administered every other week (q2w).

In certain exemplary embodiments, the antibody or antigen-binding fragment thereof is administered subcutaneously. In certain exemplary embodiments, the antibody or antigen-binding fragment thereof is administered subcutaneously using an autoinjector, a needle and syringe, or a pen delivery device.

According to another aspect, a method for treating allergic asthma in a subject in need thereof comprising administering to the subject an initial dose of about 600 mg of an antibody or antigen-binding fragment thereof that specifically binds interleukin-4R (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, and one or more subsequent doses of about 300 mg of the antibody or antigen-binding fragment thereof, is provided. In another aspect, an initial dose of about 600 mg of an antibody or antigen-binding fragment thereof that specifically binds interleukin-4R (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, and one or more subsequent doses of about 300 mg of the antibody or antigen-binding fragment thereof, is provided for use to treat allergic asthma in a subject in need thereof.

In certain exemplary embodiments, the antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28.

According to another aspect, a method for treating allergic asthma in a subject in need thereof comprising administering to the subject a first antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, and a second antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, a first antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, and a second antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to treat allergic asthma in a subject in need thereof.

In certain exemplary embodiments, the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the first antibody or antigen-binding fragment thereof comprises REGN3500.

In certain exemplary embodiments, the second antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the second antibody or antigen-binding fragment thereof comprises dupilumab.

In certain exemplary embodiments, the second antibody or antigen binding fragment thereof is administered at a dose of about 0.1 mg to about 600 mg, about 100 mg to about 400 mg, or about 300 mg. In certain exemplary embodiments, the second antibody or antigen binding fragment thereof is administered at an initial dose of about 600 mg. In certain exemplary embodiments, the second antibody or antigen binding fragment thereof is administered in one or more subsequent doses of about 300 mg of the antibody or antigen binding fragment thereof.

In certain exemplary embodiments, the second antibody or antigen-binding fragment thereof is administered every week (q1w), every other week (q2w), once every three weeks (q3w), or once every four weeks (q4w). In certain exemplary embodiments, the second antibody or antigen-binding fragment thereof is administered every other week (q2w).

In certain exemplary embodiments, the second antibody or antigen-binding fragment thereof is administered subcutaneously. In certain exemplary embodiments, the antibody or antigen-binding fragment thereof is administered subcutaneously using an autoinjector, a needle and syringe, or a pen delivery device.

In certain exemplary embodiments, the first antibody or antigen-binding fragment thereof is administered intravenously at a dose of 10 mg/kg. In certain exemplary embodiments, the first antibody or antigen binding fragment thereof is administered subcutaneously at a dose of about 0.1 mg to about 600 mg, about 100 mg to about 400 mg, or about 300 mg. In certain exemplary embodiments, the first antibody or antigen binding fragment thereof is administered subcutaneously at an initial dose of about 600 mg or about 300 mg. In certain exemplary embodiments, the first antibody or antigen binding fragment thereof is administered subcutaneously in one or more secondary doses of about 300 mg.

According to another aspect, a method for treating allergic asthma in a subject in need thereof comprising administering to the subject a first antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, wherein the first antibody or antigen-binding fragment thereof is administered at a single dose of 10 mg/kg, and a second antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, wherein the second antibody or antigen-binding fragment thereof is administered at an initial dose of 600 mg and one or more subsequent doses of about 300 mg, is provided. In one aspect, a first antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, wherein the first antibody or antigen-binding fragment thereof is administered at a single dose of 10 mg/kg, and a second antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, wherein the second antibody or antigen-binding fragment thereof is administered at an initial dose of 600 mg and one or more subsequent doses of about 300 mg, is provided for use to treat allergic asthma in a subject in need thereof.

In certain exemplary embodiments, the allergic asthma is mild allergic asthma. In certain exemplary embodiments, the allergic asthma is mild persistent allergic asthma.

In certain exemplary embodiments, the subject is allergic to house dust mite allergen (HDM). In certain exemplary embodiments, the subject is a non-smoker. In certain exemplary embodiments, the subject is clinically stable and requires short-acting inhaled β2 agonist (SABA) use on a per needed basis to control asthma symptoms.

In certain exemplary embodiments, loss of asthma control (LOAC) is reduced in the subject. In certain exemplary embodiments, an asthma symptom selected from the group consisting of cough, wheezing, and short-acting inhaled β2 agonist use is reduced in the subject.

In certain exemplary embodiments, one or more asthma-associated parameter(s) are improved in the subject. In certain exemplary embodiments, the asthma-associated parameter is selected from the group consisting of forced expiratory volume in 1 second (FEV1), peak expiratory flow (PEF), forced vital capacity (FVC), forced expiratory flow (FEF) 25%-75%, and reduction of the frequency or the dosage of short-acting inhaled β2 agonist use in the subject. In certain exemplary embodiments, pre-bronchodilator FEV1 is improved in the subject.

In certain exemplary embodiments, blood eosinophil levels are reduced in the subject.

In certain exemplary embodiments, one or both of asthma control questionnaire 5-question version (ACQ-5) score and asthma quality of life questionnaire with standardized activities (AQLQ) score are improved in the subject.

In certain exemplary embodiments, the frequency or the dosage of SABA use in the subject is reduced.

In certain exemplary embodiments, BAC-induced lung inflammation is reduced in the subject.

In certain exemplary embodiments, level of a type 2 cytokine is decreased in the subject. In certain exemplary embodiments, the type 2 cytokine is selected from the group consisting of IL-13 and IL-5. In certain exemplary embodiments, the level of the type 2 cytokine is measured by determining an mRNA level of one or more type 2 mediator genes, and wherein the mRNA level is decreased by at least about 50%, 60%, 70%, 80%, or 90%. In certain exemplary embodiments, a level of a cytokine or chemokine selected from the group consisting of tumor necrosis factor-alpha (TNFα), TARC, pulmonary and activation-regulated chemokine (PARC), CCL1, CCL26, FCER2, SIGLEC8, CCL17, and eotaxin-3 is decreased in the subject.

In certain exemplary embodiments, early allergen response (EAR) or late allergen response (LAR) is reduced in the subject. In certain exemplary embodiments, FEV1 is improved in the subject by at least 20%, 30%, 40%, 50%, 60%, or 70%. In certain exemplary embodiments, FeNO levels are reduced in the subject. In certain exemplary embodiments, serum levels of sST2, IL-33, calcitonin, or matrix metalloproteinase-12 (MMP12) are reduced in the subject. In certain exemplary embodiments, serum levels of CCL26, CCL17, or SIGLEC8 are reduced in the subject. In certain exemplary embodiments, serum levels of ASAP1-IT1, AX747757, BC042385, PABPC1P2, AB209315, AX748268, TCEAL5, CCL13, CLC, CACNG8, GPR82, GATA1, PRSS33, FFAR3, LGALS12, ASB2, PTGDR2, IL-13, IL-5, PTGDS, or RD3 are reduced in the subject.

According to another aspect, a method for reducing a cytokine level or a chemokine level in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided for use to reduce a cytokine level or a chemokine level in a subject having allergic asthma.

In certain exemplary embodiments, the cytokine is one or both of IL-13 and IL-5. In certain exemplary embodiments, the cytokine or chemokine is selected from the group consisting of TNFα, TARC, PARC, CCL1, CCL26, FCER2, SIGLEC8, CCL17 and eotaxin-3.

In certain exemplary embodiments, serum levels of sST2, IL-33, calcitonin or MMP12 are reduced in the subject. In certain exemplary embodiments, serum levels of CCL26, CCL17 or SIGLEC8 are reduced in the subject.

In certain exemplary embodiments, the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the anti-IL-33 antibody or antigen-binding fragment thereof comprises a heavy chain comprising the amino acid sequence of SEQ ID NO; 18 and a light chain comprising the amino acid sequence of SEQ ID NO: 20.

In certain exemplary embodiments, the method further comprises administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

According to another aspect, a method for reducing expression of one or more allergic asthma signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided to reduce expression of one or more allergic asthma signature genes in a subject having allergic asthma.

In certain exemplary embodiments, the one or more allergic asthma signature genes are selected from the group consisting of BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, P1K3R6, LTC4S, CLEC11A, TRABD2A, DLGAP3, VDR, DKFZp686M11215, SIGLEC12, BC016361, BC052769, and RHOH. In certain exemplary embodiments, the one or more allergic asthma signature genes are selected from the group consisting of ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS and RD3.

In certain exemplary embodiments, the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the anti-IL-33 antibody or antigen-binding fragment thereof comprises a heavy chain comprising the amino acid sequence of SEQ ID NO; 18 and a light chain comprising the amino acid sequence of SEQ ID NO: 20.

In certain exemplary embodiments, the method further comprises administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

According to another aspect, a method for reducing expression of any combination of type 2 inflammatory cytokine and type 2 chemokine signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided to reduce expression of any combination of type 2 inflammatory cytokine and type 2 chemokine signature genes in a subject having allergic asthma.

In certain exemplary embodiments, the type 2 inflammatory cytokine and chemokine signature genes are selected from the group consisting of IL-5, CCL1, IL-13, GATA2, CCL26, FCER2, CACNG8, CLC, GATA1, LGALS12, SIGLEC8, GGT5, CCL17 and MMP10. In certain exemplary embodiments, the one or more type 2 inflammatory cytokine and chemokine signature genes are selected from the group consisting of IL-5, CCL1, IL-13, CCL26, FCER2, SIGLEC8, GGT5 and CCL17.

In certain exemplary embodiments, the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the anti-IL-33 antibody or antigen-binding fragment thereof comprises a heavy chain comprising the amino acid sequence of SEQ ID NO; 18 and a light chain comprising the amino acid sequence of SEQ ID NO: 20.

In certain exemplary embodiments, the method further comprises administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

According to another aspect, a method for reducing expression of one or more eosinophil signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided for use to reduce expression of one or more eosinophil signature genes in a subject having allergic asthma.

In certain exemplary embodiments, the one or more one or more eosinophil signature genes are selected from the group consisting of IL1RL1, ADARB1, SIGLEC8, ASB2, VSTM1, SYNE1, CLC, PTPN7 and HDC.

In certain exemplary embodiments, the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the anti-IL-33 antibody or antigen-binding fragment thereof comprises a heavy chain comprising the amino acid sequence of SEQ ID NO; 18 and a light chain comprising the amino acid sequence of SEQ ID NO: 20.

In certain exemplary embodiments, the method further comprises administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

According to another aspect, a method for reducing expression of one or more type 2 inflammatory signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided. In one aspect, an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16, is provided for use to reduce expression of one or more type 2 inflammatory signature genes in a subject having allergic asthma.

In certain exemplary embodiments, the one or more type 2 inflammatory signature genes are selected from the group consisting of IL-4, IL-13, CCL26, CCL13, CCL17. CCL11, POSTN, IL-5 and IL-9.

In certain exemplary embodiments, the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the anti-IL-33 antibody or antigen-binding fragment thereof comprises a heavy chain comprising the amino acid sequence of SEQ ID NO; 18 and a light chain comprising the amino acid sequence of SEQ ID NO: 20.

In certain exemplary embodiments, the method further comprises administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

According to another aspect, a method for reducing a cytokine level or a chemokine level in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to reduce a cytokine level or a chemokine level in a subject having allergic asthma.

In certain exemplary embodiments, the cytokine is one or both of IL-13 and IL-5.

In certain exemplary embodiments, the cytokine or chemokine is selected from the group consisting of TNFα, TARC, PARC, CCL1, CCL26, FCER2, SIGLEC8, CCL17 and eotaxin-3.

In certain exemplary embodiments, serum levels of sST2, IL-33, calcitonin or MMP12 are reduced in the subject. In certain exemplary embodiments, serum levels of CCL26, CCL17 or SIGLEC8 are reduced in the subject.

In certain exemplary embodiments, the antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the method further comprises administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

According to another aspect, a method for reducing expression of one or more allergic asthma signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to reduce expression of one or more allergic asthma signature genes in a subject having allergic asthma.

In certain exemplary embodiments, the one or more allergic asthma signature genes are selected from the group consisting of BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, P1K3R6, LTC4S, CLEC11A, TRABD2A, DLGAP3, VDR, DKFZp686M11215, SIGLEC12, BC016361, BC052769, and RHOH. In certain exemplary embodiments, the one or more allergic asthma signature genes are selected from the group consisting of ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS and RD3.

In certain exemplary embodiments, the antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the method further comprises administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

According to another aspect, a method for reducing expression of any combination of type 2 inflammatory cytokine and type 2 chemokine signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to reduce any combination of type 2 inflammatory cytokine and type 2 chemokine signature genes in a subject having allergic asthma.

In certain exemplary embodiments, the type 2 inflammatory cytokine and chemokine signature genes are selected from the group consisting of IL-5, CCL1, IL-13, GATA2, CCL26, FCER2, CACNG8, CLC, GATA1, LGALS12, SIGLEC8, GGT5, CCL17 and MMP10. In certain exemplary embodiments, the one or more type 2 inflammatory cytokine and chemokine signature genes are selected from the group consisting of IL-5, CCL1, IL-13, CCL26, FCER2, SIGLEC8, GGT5 and CCL17.

In certain exemplary embodiments, the antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the method further comprises administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

According to another aspect, a method for reducing expression of one or more eosinophil signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to reduce expression of one or more eosinophil signature genes in a subject having allergic asthma.

In certain exemplary embodiments, wherein the one or more one or more eosinophil signature genes are selected from the group consisting of IL1RL1, ADARB1, SIGLEC8, ASB2, VSTM1, SYNE1, CLC, PTPN7 and HDC.

In certain exemplary embodiments, the antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the method further comprises administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

According to another aspect, a method for reducing expression of one or more type 2 inflammatory signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided. In one aspect, an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26, is provided for use to reduce expression of one or more type 2 inflammatory signature genes in a subject having allergic asthma.

In certain exemplary embodiments, the one or more type 2 inflammatory signature genes are selected from the group consisting of IL-4, IL-13, CCL26, CCL13, CCL17. CCL11, POSTN, IL-5 and IL-9.

In certain exemplary embodiments, the antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the antibody or antigen-binding fragment thereof comprises dupilumab.

In certain exemplary embodiments, the method further comprises administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

### BRIEF DESCRIPTION OF THE FIGURES

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 depicts a diagram of the 8-week study designed to assess the effect of treatment on bronchial allergen challenge (BAC)-induced airway inflammation. The flow chart depicts the following events: induced sputum collection at baseline (just before BAC) and post-BAC (8 and 24 hours after BAC), at screening (before treatment), and at week 4 and week 8 after treatment initiation. The BAC-induced change in sputum inflammatory signature is evaluated by comparing the sputum signature at baseline and post-BAC at screening (screening change), at week 4 (week 4 change) and at week 8 (week 8 change) after treatment initiation. The effect of treatment in sputum inflammatory signature is evaluated by assessing the difference between the BAC-induced screening change and the BAC-induced week 4 change (screening to week 4 change) and the difference between the BAC-induced screening change and the BAC-induced week 8 change (screening to week 8 change).

FIG. 2 depicts a diagram of part 1 of the study. Eligible patients (up to 32 in total) are randomized 1:1:1:1 to receive REGN3500, dupilumab, REGN3500 plus dupilumab combination, or placebo.

FIG. 3 depicts a diagram of part 2 of the study. Approximately 6 patients receive fluticasone propionate inhaled 500 µg (2 puffs of 250 µg) per dose, twice per day for 4 days (total 8 doses), starting on day 1.

FIG. 4 depicts a diagram of the mechanism of action of IL-33 as an initiator and amplifier of innate and adaptive immunity. As depicted in FIG. 4, IL-33 is released following tissue damage.

FIG. 5 presents data showing that treatment with anti-IL-33 reduces inflammation in a chronic house dust mite (HDM) model of lung inflammation. This figure shows that treatment with anti-IL-33 suppresses pro-inflammatory cytokines and chemokines. Data is presented showing levels of lung eosinophils and lung neutrophils in the HDM model with and without anti-IL-33 treatment. A heat map of a lung cytokine gene panel is presented showing levels of hIL-4, IL-5, IL-1b, TNFα, IFNg, GROa, and MCP-1. Alveolar SMA testing data is presented as well.

FIG. 6 depicts a diagram of the mechanism of action of anti-IL33 in reducing Type 1 and Type 2 Inflammation. As demonstrated in this figure, IL-33 drives persistent and exacerbating lung inflammation and remodeling. Anti-IL-33 reduces multiple components of chronic HDM-driven lung inflammation.

FIG. 7 depicts a schematic of the study of Example 1, described herein. This figure shows the steps of bronchial allergen challenge in mild asthmatics. Subjects are treated with placebo, placebo and REGN3500, dupilumab and placebo, or dupilumab and REGN3500. The effect of the respective treatments is determined by RNA sequencing of sputum cells following patient inhalation of hypertonic saline expectorate sputum.

FIG. 8 depicts the expression of various signature genes related to type 2 inflammation before allergen challenge, 8 hours after the allergen challenge, and 24 hours after the allergen challenge. These results show that the top genes induced by the bronchial allergen challenge at screening are enriched for type 2 inflammation, and the particular genes of interest include, but are not limited to, IL-4, IL-5, IL-13, IL-9, IL1RL1 (IL-33 receptor), Eot-3 (CCL26), TARC (CCL17), and FCER2. The list was derived by sorting on mean relative change greater than 10-fold, FDR less than 0.05. The allergen challenge signal was reproducible, but the magnitude differed among groups. Top genes identified (listed from top to bottom) included MMP10, WNT5A, CO1B, CD1A, CCL1, CCL17, PPP1R14A, IL-9, IL-5, IL-13, FCER2, CCL26, K3AA1755, GGT5, SIGLEC8, LGALS12, GATA1, CLC, CACNG8, BC015656, AKX05132, FFAR3, CACH1, IL1RL1, HPH4, CC5AML, GATA2, TAL1, HDC, NTRX1, IL-4.

FIG. 9 depicts the top type 2 inflammatory cytokine and chemokine signature genes induced by allergen challenge at 8 or 24 hours (FC greater than or equal to 12 or p(adj) less than or equal to 0.05) and suppressed by REGN3500. These results show that REGN3500 suppressed type 2 inflammatory cytokines and chemokines including IL-5, IL-13, TARC, and Eotaxin-3. Other genes of interest suppressed by REGN3 500 and induced by the bronchial allergen challenge included CCL1, a ligand for CCR8 that attracts activated Th2 type and Treg cells, CCL26, FCER2, SIGLEC8, and CCL17.

FIG. 10 depicts an eosinophil gene signature utilized to evaluate treatment effects on sputum eosinophil levels. A set of 10 genes showed high correlation to eosinophil counts in sputum at both pre- and post-allergen challenge. This gene set includes ADARB1, ASB2, CLC, GLOD5, HDC, IL1RL1, PTPN7, SIGLEC8, SYNE1, and VSTM1. An mRNA signature improved statistical performance to detect treatment effect size (fluticasone), over sputum % eosinophils. Genes were not exclusive to eosinophils, e.g., SIGLEC8 (expressed in eosinophils, basophils and mast cells), HDC (expressed in mast cells), and VSTM1 (expressed in myeloid cells).

FIG. 11 depicts the effects of REGN3500 on eosinophil signature genes in sputum, showing a suppression of eosinophil signature genes. FIG. 11 also shows that REGN3500 did not have an effect on neutrophil signature genes. Data is presented for the genes ADARB1, ASB2, CLC, HDC, IL1RL1, PTPN7, SIGLEC8, SYNE1, and VSTM1.

FIG. 12 depicts the effects of REGN3500 on a type 2 inflammatory signature genes in sputum, showing a suppression of type 2 inflammatory signature genes. FIG. 12 also shows that type 1 inflammatory signature genes were not induced by the allergen challenge. Data is presented for IL4, IL13, CCL26, CCL13, CCL17, CCL11, POSTN, IL5, and IL9.

FIG. 13A-FIG. 13B depict data showing the mechanism of action of increase IL-33 levels in driving a self-perpetuating amplification loop that primes tissues for exacerbations. FIG. 13A -FIG. 13B includes data obtained in the HDM model. Increased IL-33 levels drove a self-perpetuating amplification loop that primed tissues for exacerbations.

FIG. 14 depicts eosinophil gene signature scores across treatment arms. The arms include placebo, fluticasone, dupilumab, REGN3500, and the combination therapy of dupilumab and REGN3500. Results are presented pre- and post-bronchial allergen challenge. These results show that both dupilumab and REGN3500 were able to reduce eosinophil gene signature scores post bronchial allergen challenge. The combination treatment of dupilumab and REGN3500 was the most effective treatment in reducing eosinophil gene signature scores post bronchial allergen challenge.

FIG. 15 depicts Type 2 signature scores across the treatment arms. The arms include placebo, fluticasone, dupilumab, REGN3500, and the combination therapy of dupilumab and REGN3500. Results are presented pre- and post-bronchial allergen challenge. These results show a lower decrease of Type 2 signature scores in the REGN3500 treatment arm than the fluticasone treatment arm.

FIG. 16 depicts allergic asthma signature genes affected by REGN3500 (at 8 and/or 24 hours). Results are presented at screening and at treatment, which occurred post-bronchial allergen challenge. Genes tested included, from top to bottom, BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, P1K3R6, LTC4S, CLEC11A, TRABD2A, DLGAP3, VDR, DKFZp686M11215, SIGLEC12, BC016361, BC052769, and RHOH.

FIG. 17 depicts the top allergic asthma signature genes induced by the bronchial allergen challenge at 24 hours and suppressed by REGN3500. Results are presented at screening and at treatment, which occurred post-bronchial allergen challenge. The genes depicted include, from top to bottom, ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS, and RD3.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the invention, the typical methods and materials are now described. All publications mentioned herein are incorporated herein by reference in their entirety.

### Methods for Reducing the Incidence of Allergic Asthma Exacerbations

The invention includes methods for reducing the incidence of allergic asthma exacerbations in a subject in need thereof comprising administering a pharmaceutical composition comprising an interleukin-33 (IL-33) antagonist. Also provided is an interleukin-33 (IL-33) antagonist for use to reduce the incidence of allergic asthma exacerbations in a subject in need thereof. The invention also includes methods for reducing the incidence of allergic asthma exacerbations in a subject in need thereof comprising administering a pharmaceutical composition comprising an interleukin-4 receptor (IL-4R) antagonist. Also provided is an interleukin-4 receptor (IL-4R) antagonist for use to reduce the incidence of allergic asthma exacerbations in a subject in need thereof. The methods featured in the invention further comprise administering to a subject in need thereof a first therapeutic composition comprising an interleukin-33 (IL-33) antagonist, and a second therapeutic composition comprising an interleukin-4 receptor (IL-4R) antagonist. Also provided is an interleukin-33 (IL-33) antagonist and an interleukin-4 receptor (IL-4R) antagonist for use to reduce the incidence of allergic asthma exacerbations in a subject in need thereof. According to certain embodiments, the IL-33 antagonist is an antibody or antigen-binding fragment thereof that specifically binds IL-33. Exemplary anti-IL-33 antibodies that can be used in the context of the methods or uses featured in the invention are described herein. According to certain embodiments, the IL-4R antagonist is an antibody or antigen-binding fragment thereof that specifically binds IL-4R. Exemplary anti-IL-4R antibodies that can be used in the context of the methods or uses featured in the invention are described herein.

As used herein, the expression “asthma exacerbation” means an increase in the severity and/or frequency and/or duration of one or more symptoms or indicia of asthma. An “asthma exacerbation” also includes any deterioration in the respiratory health of a subject that requires and or is treatable by a therapeutic intervention for asthma (such as, e.g., steroid treatment, inhaled corticosteroid treatment, hospitalization, etc.). There are two types of asthma exacerbation events: a loss of asthma control (LOAC) event and a severe exacerbation event.

According to certain embodiments, a loss of asthma control (LOAC) event is defined as one or more of the following: (a) 30% or greater reduction from baseline in morning PEF on 2 consecutive days; (b) greater than or equal to 6 additional reliever puffs of salbutamol/albuterol or levosalbutamol/levalbuterol in a 24-hour period (compared to baseline) on 2 consecutive days; (c) an increase in ICS greater than or equal to 4 times the last prescribed ICS dose (or ≥50% of the prescribed ICS dose at V2 if background therapy withdrawal completed); (d) use of systemic (oral and/or parenteral) steroid treatment; or (e) hospitalization or emergency room visit because of asthma.

In certain instances, an asthma exacerbation may be categorized as a “severe asthma exacerbation event.” A severe asthma exacerbation event means an incident requiring immediate intervention in the form of treatment with either systemic corticosteroids or with inhaled corticosteroids at four or more times the dose taken prior to the incident. According to certain embodiments, a severe asthma exacerbation event is defined as a deterioration of asthma requiring: use of systemic corticosteroids for greater than or equal to 3 days; or hospitalization or emergency room visit because of asthma, requiring systemic corticosteroids. The general expression “asthma exacerbation” therefore includes and encompasses the more specific subcategory of “severe asthma exacerbations.” Accordingly, methods for reducing the incidence of severe asthma exacerbations in a patient in need thereof are included.

A “reduction in the incidence” of an asthma exacerbation means that a subject who has received a pharmaceutical composition comprising an IL-4R antagonist experiences fewer allergic asthma exacerbations (i.e., at least one fewer exacerbation) after treatment than before treatment, or experiences no allergic asthma exacerbations for at least 4 weeks (e.g., 4, 6, 8, 12, 14, or more weeks) following initiation of treatment with the pharmaceutical composition. A “reduction in the incidence” of an asthma exacerbation alternatively means that, following administration of the pharmaceutical composition, the likelihood that a subject experiences an asthma exacerbation is decreased by at least 10% (e.g., 10%, 15%, 20%, 25%, 30%, 35%, 40%, 45%, 50%, or more) as compared to a subject who has not received the pharmaceutical composition.

The invention includes methods for reducing the incidence of allergic asthma exacerbations in a subject in need thereof comprising administering a pharmaceutical composition comprising an IL-4R antagonist to the subject as well as administering to the subject one or more maintenance doses of an inhaled corticosteroid (ICS) and/or one or more maintenance doses of a second controller, e.g., a long-acting beta-agonist (LABA) or a leukotriene receptor antagonist (LTA). Also provided is a pharmaceutical composition comprising an interleukin-4 receptor (IL-4R) antagonist for use, in combination with one or more maintenance doses of an inhaled corticosteroid (ICS) and/or one or more maintenance doses of a second controller, e.g., a long-acting beta-agonist (LABA) or a leukotriene receptor antagonist (LTA), to reduce the incidence of allergic asthma exacerbations in a subject in need thereof. The invention includes methods for reducing the incidence of allergic asthma exacerbations in a subject in need thereof comprising administering a pharmaceutical composition comprising an IL-33 antagonist to the subject as well as administering to the subject one or more maintenance doses of an inhaled corticosteroid (ICS) and/or one or more maintenance doses of a second controller, e.g., a long-acting beta-agonist (LABA) or a leukotriene receptor antagonist (LTA). Also provided is a pharmaceutical composition comprising an interleukin-33 (IL-33) antagonist for use, in combination with one or more maintenance doses of an inhaled corticosteroid (ICS) and/or one or more maintenance doses of a second controller, e.g., a long-acting beta-agonist (LABA) or a leukotriene receptor antagonist (LTA), to reduce the incidence of allergic asthma exacerbations in a subject in need thereof. Suitable ICSs include, but are not limited to, fluticasone (e.g., fluticasone propionate, e.g., Flovent™), budesonide, mometasone (e.g., mometasone furoate, e.g., Asmanex™), flunisolide (e.g., Aerobid™), dexamethasone acetate/phenobarbital/theophylline (e.g., Azmacort™), beclomethasone dipropionate HFA (Qvar™), and the like. Suitable LABAs include, but are not limited to, salmeterol (e.g., Serevent™), formoterol (e.g., Foradil™), and the like. Suitable LTAs include, but are not limited to, montelukast (e.g., Singulaire™), zafirlukast (e.g., Accolate™), and the like.

The invention includes methods for reducing the incidence of allergic asthma exacerbations in a subject in need thereof comprising administering a pharmaceutical composition comprising one or both of an IL-4R antagonist and an IL-33 antagonist to the subject as well as administering to the subject one or more reliever medications to eliminate or reduce one or more asthma-associated symptoms. Also provided is a pharmaceutical composition comprising one or both of an IL-4R antagonist and an IL-33 antagonist for use, in combination with one or more maintenance doses of an inhaled corticosteroid (ICS) and/or one or more maintenance doses of a second controller, e.g., a long-acting beta-agonist (LABA) or a leukotriene receptor antagonist (LTA), to reduce the incidence of allergic asthma exacerbations in a subject in need thereof. The invention includes methods for reducing the incidence of allergic asthma exacerbations in a subject in need thereof comprising administering a pharmaceutical composition comprising one or both of an IL-4R antagonist and an IL-33 antagonist to the subject as well as administering to the subject one or more reliever medications to eliminate or reduce one or more asthma-associated symptoms. Also provided is a pharmaceutical composition comprising one or both of an IL-4R antagonist and an IL-33 antagonist for use, in combination with one or more reliever medications to eliminate or reduce one of more asthma-associated symptoms, to reduce the incidence of allergic asthma exacerbations in a subject in need thereof. Suitable reliever medications include, but are not limited to, quick-acting beta₂-adrenergic receptor agonists such as, e.g., albuterol (i.e., salbutamol, e.g., Proventil™, Ventolin™, Xopenex™ and the like), pirbuterol (e.g., Maxair™), metaproterenol (e.g., Alupent™) and the like.

### Methods for Improving Asthma-Associated Parameters

The invention also includes methods for improving one or more asthma-associated parameters in a subject in need thereof, wherein the methods comprise administering a pharmaceutical composition comprising an IL-33 antagonist to the subject. Also provided is a pharmaceutical composition comprising an IL-33 antagonist for use in improving one or more asthma associated parameters in a subject in need thereof. The invention additionally includes methods for improving one or more asthma-associated parameters in a subject in need thereof, wherein the methods comprise administering a pharmaceutical composition comprising an IL-4R antagonist to the subject. Also provided is a pharmaceutical composition comprising an IL-4R antagonist for use in improving one or more asthma associated parameters in a subject in need thereof. The invention also includes methods for improving one or more asthma-associated parameters in a subject in need thereof, wherein the methods comprise administering a first pharmaceutical composition comprising an IL-33 antagonist and a second pharmaceutical composition comprising an IL-4R antagonist to the subject. Also provided is a first pharmaceutical composition comprising an IL-33 antagonist and a second pharmaceutical composition comprising an IL-4R antagonist for use in improving one or more asthma associated parameters in a subject in need thereof. A reduction in the incidence of an asthma exacerbation (as described above) may correlate with an improvement in one or more asthma-associated parameters; however, such a correlation is not necessarily observed in all cases.

Examples of “asthma-associated parameters” include: (1) relative percent change from baseline (e.g., at week 12) in forced expiratory volume in 1 second (FEV₁); (2) a relative percent change from baseline (e.g., at week 12) as measured by forced expiratory flow at 25-75% of the pulmonary volume (FEF25-75); (3) annualized rate of loss of asthma control events during the treatment period; (4) annualized rate of severe exacerbation events during the treatment period; (5) time to loss of asthma control events during the treatment period; (6) time to severe exacerbation events during the treatment period; (7) time to loss of asthma control events during overall study period; (8) time to severe exacerbation events during overall study period; (9) health care resource utilization; (10) change from baseline at week 12 in: i) morning and evening asthma symptom scores, ii) ACQ-5 score, iii) AQLQ score, iv) morning and evening PEF, v) number of inhalations/day of salbutamol/albuterol or levosalbutamol/levalbuterol for symptom relief, vi) nocturnal awakenings; (11) change from baseline at week 12 and week 24 in: i) 22-item Sino Nasal Outcome Test (SNOT-22), ii) Hospital Anxiety and Depression Score (HADS), iii) EuroQual questionnaire (EQ-5D-3L or EQ-5D-5L). An “improvement in an asthma-associated parameter” means an increase from baseline of one or more of FEV₁, AM PEF or PM PEF, and/or a decrease from baseline of one or more of daily albuterol/levalbuterol use, ACQ5 score, average nighttime awakenings or SNOT-22 score. As used herein, the term “baseline,” with regard to an asthma-associated parameter, means the numerical value of the asthma-associated parameter for a patient prior to or at the time of administration of a pharmaceutical composition comprising an IL-33 antagonist, the numerical value of the asthma-associated parameter for a patient prior to or at the time of administration of a pharmaceutical composition comprising an IL-4R antagonist, or the numerical value of the asthma-associated parameter for a patient prior to or at the time of administration of a first pharmaceutical composition comprising an IL-33 antagonist and a second pharmaceutical composition comprising an IL-4R antagonist to the subject.

To determine whether an asthma-associated parameter has “improved,” the parameter is quantified at baseline and at a time point after administration of the pharmaceutical composition described herein. For example, an asthma-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the asthma associated parameter (e.g., an increase or decrease, as the case may be, depending on the specific parameter being measured).

“Asthma-associated parameters” also include altered (i.e., increased or decreased) expression of one or more (e.g., 2, 3, 4, 5, 6, 7, 8, 9, 10, 11, 12, 13, 14, 15, 16, 17, 18, 19, 20 or more) allergic asthma “signature genes” compared to baseline expression. As used herein, the term “baseline,” with regard to a signature gene, means the numerical value of the expression of one or more signature genes for a patient prior to or at the time of administration of a pharmaceutical composition comprising an IL-33 antagonist, the numerical value of the expression of one or more signature genes for a patient prior to or at the time of administration of a pharmaceutical composition comprising an IL-4R antagonist, or the numerical value of the expression of one or more signature genes for a patient prior to or at the time of administration of a first pharmaceutical composition comprising an IL-33 antagonist and a second pharmaceutical composition comprising an IL-4R antagonist to the subject. According to certain exemplary embodiments, a patient is selected for treatment with a pharmaceutical composition comprising an IL-33 antagonist, a pharmaceutical composition comprising an IL-4R antagonist, or a first pharmaceutical composition comprising an IL-33 antagonist and a second pharmaceutical composition comprising an IL-4R antagonist based upon increased or decreased expression of one or more signature genes.

In certain exemplary embodiments, expression of one or more signature genes is decreased compared to baseline expression levels, e.g., expression is reduced to a level that is about 99%, about 98%, about 97%, about 96%, about 95%, about 94%, about 93%, about 92%, about 91%, about 90%, about 85%, about 80%, about 75%, about 70%, about 65%, about 60%, about 55%, about 50%, about 45%, about 40%, about 35%, about 30%, about 25%, about 20%, about 15%, about 10% or about 5% of the baseline expression level, or any ranges between these numbers.

In certain exemplary embodiments, expression of one or more signature genes is increased compared to baseline expression levels, e.g., expression is reduced to a level that is about 105%, about 110%, about 120%, about 125%, about 130%, about 135%, about 140%, about 145%, about 150%, about 175%, about 200%, about 225%, about 250%, about 275%, about 300%, about 400%, or about 500% or more of the baseline expression level, or any ranges between these numbers.

Suitable signature genes include allergic asthma signature genes, which include, but are not limited to, type 2 inflammatory signature genes, cytokine signature genes, chemokine signature genes, eosinophil signature genes, and the like.

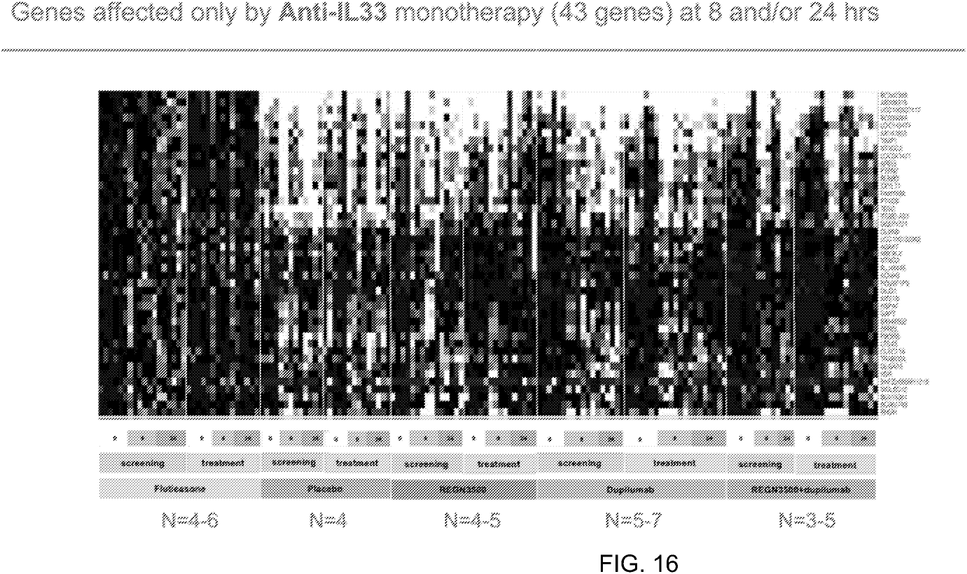

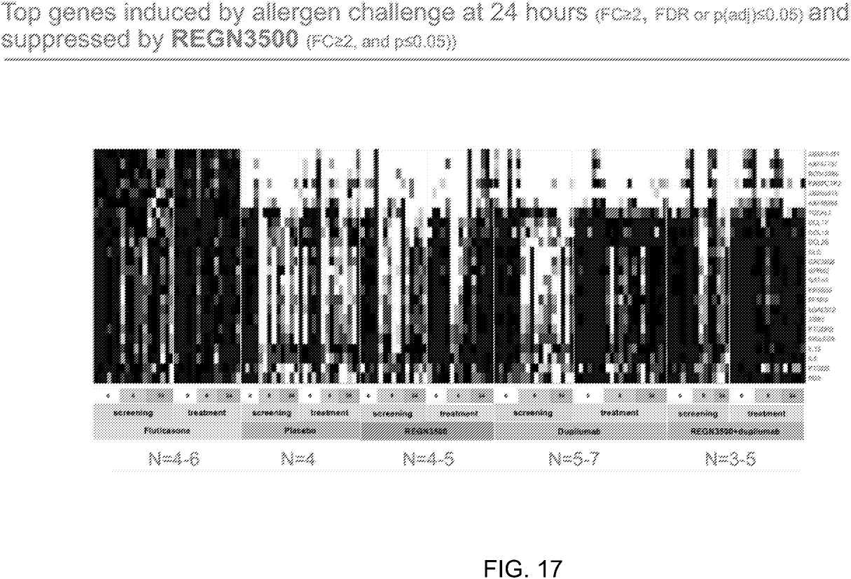

Exemplary allergic asthma signature genes are depicted at FIG. 16 and FIG. 17 and include, but are not limited to, BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, P1K3R6, LTC4S, CLEC11A, TRABD2A, DLGAP3, VDR, DKFZp686M11215, SIGLEC12, BC016361, BC052769, RHOH, ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS and RD3.

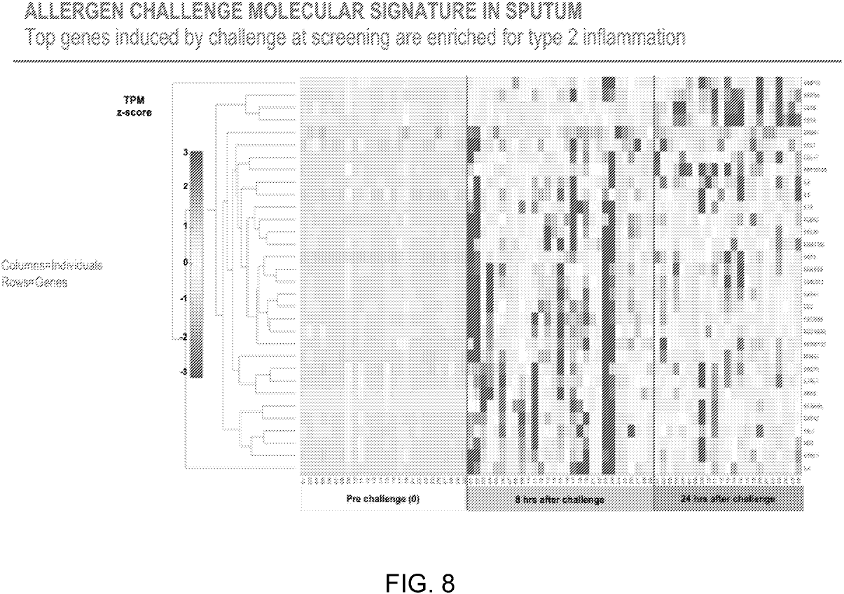

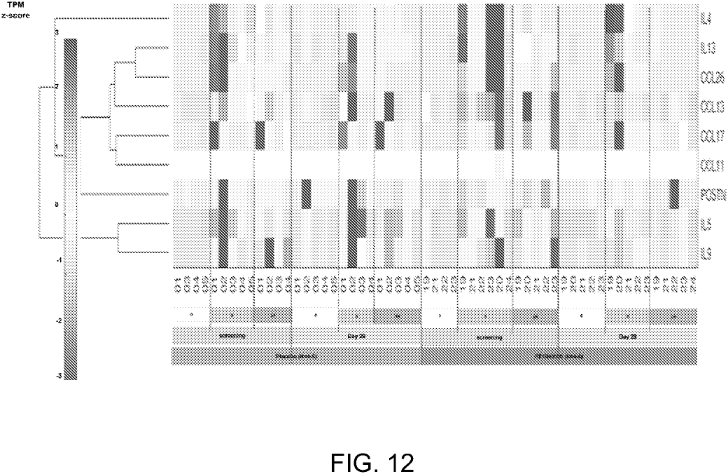

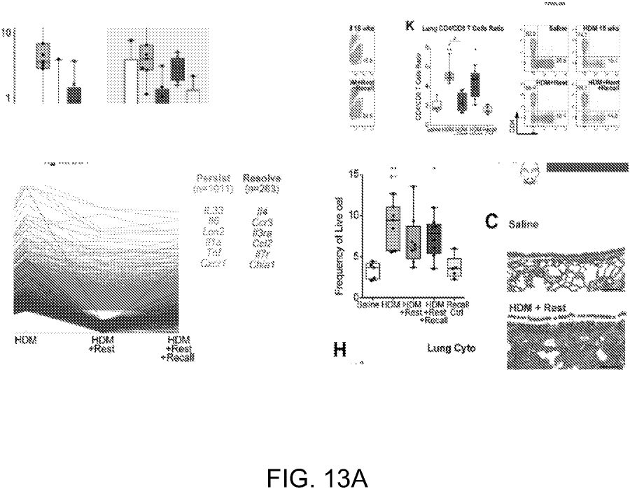

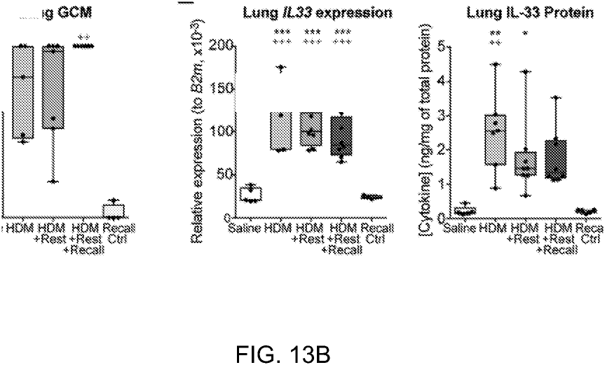

Exemplary type 2 inflammatory signature genes are depicted at FIG. 8 and FIG. 12 and include, but are not limited to, IL-4, IL-5, IL-13, IL-9, IL1RL1 (IL-33 receptor), Eot-3 (CCL26), TARC (CCL17), FCER2, MMP10, WNT5A, CO1B, CD1A, CCL1, CCL17, PPP1R14A, IL-9, IL-5, IL-13, FCER2, CCL26, K3AA1755, GGT5, SIGLEC8, LGALS12, GATA1, CLC, CACNG8, BC015656, AKX05132, FFAR3, CACH1, IL1RL1, HPH4, CC5AML, GATA2, TAL1, HDC, NTRX1, IL-4, IL4, IL13, CCL26, CCL13, CCL17, CCL11, POSTN, IL5 and IL9.

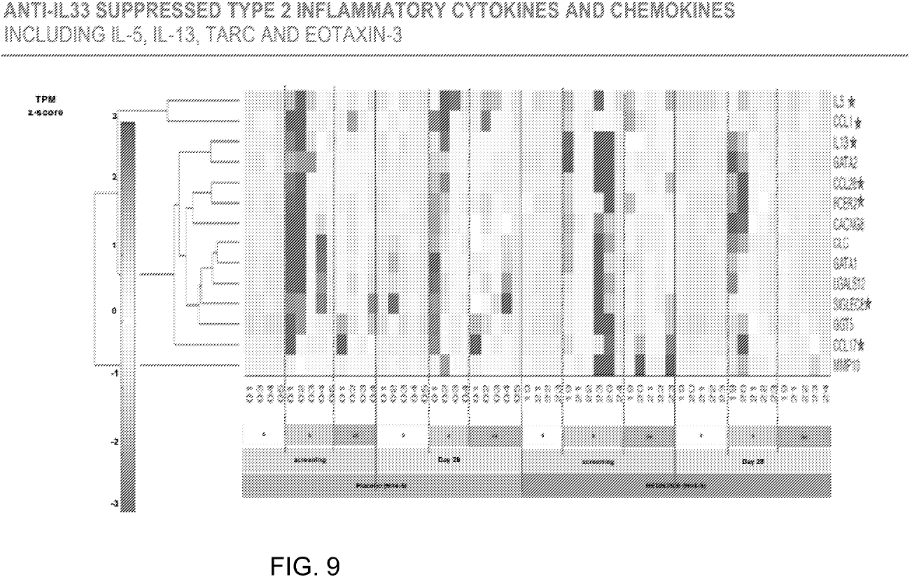

Exemplary cytokine signature genes and chemokine signature genes are depicted at FIG. 9 and include, but are not limited to, IL-5, IL-13, TARC, Eotaxin-3, CCL1, CCL26, FCER2, SIGLEC8 and CCL17.

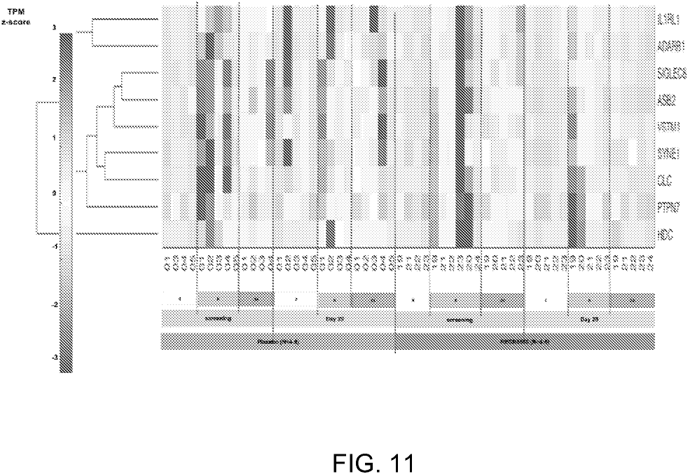

Exemplary eosinophil signature genes are depicted at FIG. 11 and include, but are not limited to, ADARB1, ASB2, CLC, HDC, IL1RL1, PTPN7, SIGLEC8, SYNE1 and VSTM1.

The level of signature genes may be detected in a biological sample using any suitable means known in the art for detecting protein, RNA (e.g., mRNA) and/or DNA, including but not limited to, Northern blotting, Western blotting, Southern blotting, immunoprecipitation, in situ hybridization, PCR (e.g., RT-PCR), array technologies (e.g., serial analysis of gene expression (SAGE), DNA microarrays, RNA seq, tiling arrays and the like), nuclease assays, and the like.

As used herein, a “biological sample” includes, but is not limited to, cell cultures or extracts thereof; biopsied material obtained from a mammal or extracts thereof; and blood, saliva, urine, feces, semen, tears, or other body fluids or extracts thereof. Exemplary biological samples include sputum and blood.

The terms “acquire” or “acquiring” as used herein, refer to obtaining possession of a physical entity, or a value, e.g., a numerical value, by “directly acquiring” or “indirectly acquiring” the physical entity or value, such as an asthma-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

### Forced Expiratory Volume in 1 Second (FEV₁)

According to certain embodiments, administration or use of an IL-4R antagonist to a patient results in an increase from baseline of forced expiratory volume in 1 second (FEV₁). In some embodiments, administration or use of an IL-33 antagonist to a patient results in an increase from baseline of forced expiratory volume in 1 second (FEV₁). In other embodiments, administration or use of an IL-4R antagonist in combination with an IL-33 (IL-33) antagonist to a patient results in an increase from baseline of forced expiratory volume in 1 second (FEV₁). Methods for measuring FEV₁ are known in the art. For example, a spirometer that meets the 2005 American Thoracic Society (ATS)/European Respiratory Society (ERS) recommendations can be used to measure FEV₁ in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV₁ (in liters).

According to certain embodiments, therapeutic methods or uses that result in an increase of FEV₁ from baseline of at least 0.05 L at week 12 following initiation of treatment with a pharmaceutical composition comprising an anti-IL-33 antagonist, a pharmaceutical composition comprising an anti-IL-4R antagonist, or a pharmaceutical composition comprising an IL-33 antagonist and an IL-4R antagonist, are provided. For example, administration of an IL-33 antagonist, an IL-4R antagonist, or an IL-33 antagonist and an IL-4R antagonist causes an increase of FEV₁ from baseline of about 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 12.

### Bronchial Allergen Challenge

According to some embodiments, administration or use of an IL-33 antagonist to a patient results in a decrease in bronchial allergen challenge (BAC)-induced lung inflammation. According to some embodiments, administration or use of an IL-4R antagonist to a patient results in a decrease in BAC-induced lung inflammation. According to some embodiments, administration or use of an IL-4R antagonist and an IL-33 antagonist to a patient results in a decrease in BAC-induced lung inflammation. The BAC is a model for testing asthma drugs and has been in use for over 30 years (Diamant et al. Inhaled allergen bronchoprovocation tests. J Allergy Clin Immunol. 2013. 132:1045-1055 e1046; Fahy et al. Analysis of cellular and biochemical constituents of induced sputum after allergen challenge: a method for studying allergic airway inflammation. J Allergy Clin Immunol. 1994. 93:1031-1039; and Inman et al. Dose-dependent effects of inhaled mometasone furoate on airway function and inflammation after allergen inhalation challenge. Am J Respir Crit Care Med. 2001. 164:569-574.) BAC involves patient inhalation of allergen that results in a biphasic airway response, which is characterized by an early (30 minutes to 2 hours post-allergen challenge) and a late (approximately 3 to 8 hours post-allergen challenge) decline in FEV1. This model facilitates the evaluation of allergic inflammatory response via measurement of changes in cell content, cytokine production, and mRNA inflammatory signatures in bronchoalveolar lavage, bronchial biopsies, or induced sputum.

### Sputum mRNA Measures

Induced sputum samples are used in clinical studies of asthma to assess airway inflammation. Studies comparing sputum from asthmatics with sputum from normal controls have found elevated concentrations of IL-33 and ST2 (Hamzaoui et al. Induced sputum levels of IL-33 and soluble ST2 in young asthmatic children. (J Asthma. 2013. 50:803-809 and Salter et al.) IL-25 and IL-33 induce Type 2 inflammation in basophils from subjects with allergic asthma. Respir Res. 2016. 17:5.), eotaxin, TARC (Heijink et al. Effect of ciclesonide treatment on allergen-induced changes in T cell regulation in asthma. Int Arch Allergy Immunol. 2008. 145:111-121 and Sekiya et al. Increased levels of a TH2-type CC chemokine thymus and activation-regulated chemokine (TARC) in serum and induced sputum of asthmatics. Allergy. 2002. 57:173-177), and both IL-5 and IL-13 (Park et al. Interleukin-13 and interleukin-5 in induced sputum of eosinophilic bronchitis: comparison with asthma. Chest. 2005. 128:1921-1927 and Peters, M.C., Z.K. Mekonnen, S. Yuan, N.R. Bhakta, P.G. Woodruff, and J.V. Fahy. Measures of gene expression in sputum cells can identify TH2-high and TH2-low subtypes of asthma. J Allergy Clin Immunol. 2014. 133:388-394) with protein and/or RNA. Sputum cytokines such as IL-4, IL-5, and IL-13 are elevated and associated with the presence of asthma symptoms and severity (Truyen et al. Evaluation of airway inflammation by quantitative Th1/Th2 cytokine mRNA measurement in sputum of asthma patients. Thorax. 2006. 61:202-208.). In previous studies, BAC in mild asthmatic patients acutely increased levels of Type 2 cytokines, such as IL-13 and IL-5 by approximately 10X in the lung. Treatment with inhaled corticosteroids significantly suppressed this BAC-mediated upregulation of protein and mRNA levels of Type 2 cytokines (Zuiker et al. Kinetics of TH2 biomarkers in sputum of asthmatics following inhaled allergen. Eur Clin Respir J. 2015. 2 and Zuiker et al. Sputum RNA signature in allergic asthmatics following allergen bronchoprovocation test. Eur Clin Respir J. 2016. 3:31324.)

According to some embodiments, administration or use of an IL-4R antagonist to a patient results in a suppression of BAC-induced upregulation of protein and/or mRNA levels of Type 2 cytokines. According to some embodiments, administration or use of an IL-33 antagonist to a patient results in a suppression of BAC-induced upregulation of protein and/or mRNA levels of Type 2 cytokines. According to some embodiments, administration or use of an IL-4R antagonist and an IL-33 antagonist to a patient results in a suppression of BAC-induced upregulation of protein and/or mRNA levels of Type 2 cytokines. According to some embodiments, administration or use of an IL-4R antagonist to a patient results in a suppression of BAC-induced upregulation of protein and/or mRNA levels of any one of CCL26, CCL17, SIGLEC8, IL-33, ST2, eotaxin, TARC, IL-4, IL-5, IL-13, ASAP1-IT1, AX747757, BC042385, PABPC1P2, AB209315, AX748268, TCEAL5, CCL13, CLC, CACNG8, GPR82, GATA1, PRSS33, FFAR3, LGALS12, ASB2, PTGDR2, PTGDS, or RD3. According to some embodiments, administration or use of an IL-33 antagonist to a patient results in a suppression of BAC-induced upregulation of protein and/or mRNA levels of any one of CCL26, CCL17, SIGLEC8, IL-33, ST2, eotaxin, TARC, IL-4, IL-5, IL-13, ASAP1-IT1, AX747757, BC042385, PABPC1P2, AB209315, AX748268, TCEAL5, CCL13, CLC, CACNG8, GPR82, GATA1, PRSS33, FFAR3, LGALS12, ASB2, PTGDR2, PTGDS, or RD3.. According to some embodiments, administration or use of an IL-4R antagonist and an IL-33 antagonist to a patient results in a suppression of BAC-induced upregulation of protein and/or mRNA levels of any one of CCL26, CCL17, SIGLEC8, IL-33, ST2, eotaxin, TARC, IL-4, IL-5, IL-13, ASAP1-IT1, AX747757, BC042385, PABPC1P2, AB209315, AX748268, TCEAL5, CCL13, CLC, CACNG8, GPR82, GATA1, PRSS33, FFAR3, LGALS12, ASB2, PTGDR2, PTGDS, or RD3..

### Sputum Cytokines and Chemokines

According to some embodiments, administration or use of an IL-4R antagonist to a patient results in a suppression of BAC-induced elevation of cytokines and chemokines related to both the IL-33 and the IL4R pathways, including IL-13, IL-5, tumor necrosis factor-alpha (TNFα), TARC, pulmonary and activation-regulated chemokine (PARC), CCL1, CCL26, FCER2, SIGLEC8, CCL17, and eotaxin-3. According to some embodiments, administration or use of an IL-33 antagonist to a patient results in a suppression of BAC-induced elevation of cytokines and chemokines related to both the IL-33 and the IL4R pathways, including IL-13, IL-5, tumor necrosis factor-alpha (TNFα), TARC, pulmonary and activation-regulated chemokine (PARC), CCL1, CCL26, FCER2, SIGLEC8, CCL17, and eotaxin-3. According to some embodiments, administration or use of an IL-4R antagonist and an IL-33 antagonist to a patient results in a suppression of BAC-induced elevation of cytokines and chemokines related to both the IL-33 and the IL4R pathways, including IL-13, IL-5, tumor necrosis factor-alpha (TNFα), TARC, pulmonary and activation-regulated chemokine (PARC), CCL1, CCL26, FCER2, SIGLEC8, CCL17, and eotaxin-3. Previous studies have shown that cytokines and chemokines may be measured in sputum induced after a BAC. Cytokines and chemokines related to both the IL-33 and the IL4R pathways, including IL-13, IL-5, tumor necrosis factor-alpha (TNFα), TARC, pulmonary and activation-regulated chemokine (PARC), CCL1, CCL26, FCER2, SIGLEC8, CCL17, and eotaxin-3, are expected to be elevated after a BAC.

### Early and Late Phase Decreased FEV1 After Bronchial Allergen Challenge

Change in pulmonary function after a BAC is a standard endpoint for most allergen challenge studies evaluating effect of inhaled corticosteroids. In sensitized patients, allergen inhalation results in an acute response characterized by bronchoconstriction within 0 to 2 hours after exposure, which is referred to as the early allergen response (EAR). This EAR is thought to represent primarily the release of preformed mast cell mediators and is typically not responsive to steroid. Early allergen response is often followed by a late allergen response (LAR) that occurs approximately 3 to 8 hours after exposure. This LAR is seen in 50% to 60% of adult asthmatic patients. The LAR coincides with the initial influx of inflammatory cells and is generally responsive to steroid. According to some embodiments, administration or use of an IL-4R antagonist to a patient results in an attenuation of BAC-induced EAR or LAR, for example as measured by FEV1. According to some embodiments, administration or use of an IL-33 antagonist to a patient results in an attenuation of BAC-induced EAR or LAR, for example as measured by FEV1. According to some embodiments, administration or use of an IL-4R antagonist and an IL-33 antagonist to a patient results in an attenuation of BAC-induced EAR or LAR, for example as measured by FEV1.

### Fractional Exhaled Nitric Oxide Measures

In a BAC, sputum eosinophils have been shown to increase in asthmatic patients who display a late phase response. Although an association of sputum eosinophils and FeNO has been reported, FeNO is not an eosinophil-specific marker and may be present in non-eosinophilic inflammation (Haldar et al. Mepolizumab and exacerbations of refractory eosinophilic asthma. N Engl J Med. 2009. 360:973-984.) Moreover, mRNA levels in bronchial tissue correlate with FeNO measures (Porsbjerg et al. IL-33 is related to innate immune activation and sensitization to HDM in mild steroid-free asthma. Clin Exp Allergy. 2016. 46:564-574).

### Serum Biomarkers

Serum levels of sST2, IL-33, calcitonin, and matrix metalloproteinase-12 (MMP12) may be increased after BAC. According to certain embodiments, administration or use of an IL-4R antagonist to a patient results in a reduction of the increase in serum levels of sST2, IL-33, calcitonin, and matrix metalloproteinase-12 (MMP12) typically seen after BAC. According to certain embodiments, administration or use of an IL-33 antagonist to a patient results in a reduction of the increase in serum levels of sST2, IL-33, calcitonin, and matrix metalloproteinase-12 (MMP12) typically seen after BAC. According to certain embodiments, administration or use of an IL-4R antagonist and an IL-33 antagonist to a patient results in a reduction of the increase in serum levels of sST2, IL-33, calcitonin, and matrix metalloproteinase-12 (MMP12) typically seen after BAC.

### FEF25-75%

According to certain embodiments, administration or use of an IL-33 antagonist, an IL-4R antagonist, or an IL-33 antagonist and an IL-4R antagonist to a patient results in an increase from baseline of FEF25-75%. Methods for measuring FEF are known in the art. For example, a spirometer that meets the 2005 American Thoracic Society (ATS)/European Respiratory Society (ERS) recommendations can be used to measure FEV₁ in a patient. The FEF25-75 (forced expiratory flow between 25% and 75%) is the speed (in liters per second) at which a person can empty the middle half of his or her air during a maximum expiration (i.e., forced vital capacity or FVC). The parameter relates to the average flow from the point at which 25 percent of the FVC has been exhaled to the point at which 75 percent of the FVC has been exhaled. The FEF25-75% of a subject provides information regarding small airway function, such that the extent of mall airway disease and/or inflammation. A change in FEF25-75 is an early indicator of obstructive lung disease. In certain embodiments, an improvement and/or increase in the FEF25-75% parameter is an improvement of at least 10%, 25%, 50% or more as compared to baseline. In certain embodiments, the methods of the invention result in normal FEF25-75% values in a subject (e.g., values ranging from 50-60% and up to 130% of the average).

### Morning and Evening Peak Expiratory Flow (AM PEF and PM PEF)

According to certain embodiments, administration or use of an IL-33 antagonist, an IL-4R antagonist, or an IL-33 antagonist and an IL-4R antagonist to a patient results in an increase from baseline of morning (AM) and/or evening (PM) peak expiratory flow (AM PEF and/or PM PEF). Methods for measuring PEF are known in the art. For example, according to one method for measuring PEF, patients are issued an electronic PEF meter for recording morning (AM) and evening (PM) PEF (as well as daily albuterol use, morning and evening asthma symptom scores, and number of nighttime awakenings due to asthma symptoms that require rescue medications). Patients are instructed on the use of the device, and written instructions on the use of the electronic PEF meter are provided to the patients. In addition, a medical professional may instruct the patients on how to record pertinent variables in the electronic PEF meter. AM PEF is generally performed within 15 minutes after arising (between 6 am and 10 am) prior to taking any albuterol. PM PEF is generally performed in the evening (between 6 pm and 10 pm) prior to taking any albuterol. Subjects should try to withhold albuterol for at least 6 hours prior to measuring their PEF. Three PEF efforts are performed by the patient and all 3 values are recorded by the electronic PEF meter. Usually the highest value is used for evaluation. Baseline AM PEF may be calculated as the mean AM measurement recorded for the 7 days prior to administration of the first dose of pharmaceutical composition comprising the IL-33 antagonist, the IL-4R antagonist, or the IL-33 antagonist and the IL-4R antagonist, and baseline PM PEF may be calculated as the mean PM measurement recorded for the 7 days prior to administration of the first dose of pharmaceutical composition comprising the IL-33 antagonist, the IL-4R antagonist, or the IL-33 antagonist and the IL-4R antagonist.

According to certain exemplary embodiments, therapeutic methods or uses that result in an increase in AM PEF and/or PM PEF from baseline of at least 1.0 L/min at week 12 following initiation of treatment with a pharmaceutical composition comprising an anti-IL-33 antagonist, an IL-4R antagonist, or an IL-33 antagonist and an IL-4R antagonist, are provided. For example, administration or use of an IL-33 antagonist, an IL-4R antagonist, or an IL-33 antagonist and an IL-4R antagonist to a subject in need thereof causes an increase in PEF from baseline of about 0.5 L/min, 1.0 L/min, 1.5 L/min, 2.0 L/min, 2.5 L/min, 3.0 L/min, 3.5 L/min, 4.0 L/min, 4.5 L/min, 5.0 L/min, 5.5 L/min, 6.0 L/min, 6.5 L/min, 7.0 L/min, 7.5 L/min, 8.0 L/min, 8.5 L/min, 9.0 L/min, 9.5 L/min, 10.0 L/min, 10.5 L/min, 11.0 L/min, 12.0 L/min, 15 L/min, 20 L/min, or more at week 12.

### Albuterol/Levalbuterol Use

According to certain embodiments, administration or use of an IL-33 antagonist, an IL-4R antagonist, or an IL-33 antagonist and an IL-4R antagonist to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of pharmaceutical composition comprising the IL-33 antagonist, the IL-4R antagonist, or the IL-33 antagonist and the IL-4R antagonist.

According to certain exemplary embodiments, therapeutic methods or uses that result in a decrease in albuterol/levalbuterol use from baseline of at least 0.25 puffs per day at week 12 following initiation of treatment with a pharmaceutical composition comprising an IL-33 antagonist, an IL-4R antagonist, or an IL-33 antagonist and an IL-4R antagonist, are provided. For example, administration or use of an IL-33 antagonist, an IL-4R antagonist, or an IL-33 antagonist and an IL-4R antagonist to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

### OCS Use

According to certain embodiments, administration or use of an IL-33 antagonist, an IL-4R antagonist, or an IL-33 antagonist and an IL-4R antagonist to a patient can be used in conjunction with an OCS such as oral prednisone. The number of OCS administrations can be recorded daily by the patients in a diary, PEF meter, or other recording device. During treatment with the pharmaceutical composition described herein, occasional short-term use of prednisone typically can be used to control acute asthmatic episodes, e.g., episodes in which bronchodilators and other antiinflammatory agents fail to control symptoms. In other aspects, prednisone is used concurrent with or as a substitution for ICS. Oral prednisone may be administered in dosages of about 5 mg, 10 mg, 15 mg, 20 mg, 25 mg, 30 mg, 35 mg or 40 mg. OCS can optionally be administered once a day or multiple times a day (e.g., twice a day, three times a day, four times a day, etc.)

In certain exemplary embodiments, methods or uses for reducing or eliminating the dependency of the subject on OCS use are provided. The reduction or elimination of steroid dependency is highly advantageous and desirable. In certain embodiments, a reduction of 50% or greater (e.g., 50%, 60%, 70%, 80%, 90% or more) in the OCS dose is achieved after administration of IL-4R antibody therapy, IL-33 antibody therapy, or IL-33 antibody therapy in combination with IL-4R antibody therapy at a period of time (e.g., at week 240). In certain embodiments, the OCS is substantially eliminated after 40 weeks, 45 weeks, 50 weeks, 52 weeks, or greater after the first dose following administration of the initial dose. In other embodiments, the level of OCS use is reduced to less than 5 mg per day (e.g., less than 5 mg, 4 mg, 3 mg, 2 mg or less per day). In other embodiments, the dependency on OCS use is substantially eliminated after 3 months, 6 months, 9 months or 1 year following treatment with IL-33 antagonist, IL-4R antagonist, or IL-33 antagonist and IL-4R antagonist.

### 5-Item Asthma Control Questionnaire (ACQ) Score

According to certain embodiments, administration or use of an IL-33 antagonist, an IL-4R antagonist, or an IL-33 antagonist and an IL-4R antagonist to a patient results in a decrease from baseline of five-item Asthma Control Questionnaire (ACQ5) score. The ACQ5 is a validated questionnaire to evaluate asthma control.

According to certain exemplary embodiments, therapeutic methods or uses that result in a decrease in ACQ5 score from baseline of at least 0.10 points at week 12 following initiation of treatment with a pharmaceutical composition comprising an IL-33 antagonist, an IL-4R antagonist, or an IL-33 antagonist and an IL-4R antagonist, are provided. For example, administration or use of an IL-33 antagonist, an IL-4R antagonist, or an IL-33 antagonist and an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 12.

### Night-Time Awakenings

According to certain embodiments, administration or use of an IL-33 antagonist, an IL-4R antagonist, or an IL-33 antagonist and an IL-4R antagonist to a patient results in a decrease from baseline of average number of nighttime awakenings.

In certain embodiments, the methods or uses decrease the average number of nighttime awakenings from baseline by at least about 0.10 times per night at week 12 following initiation of treatment. For example, administration or use of an IL-33 antagonist, an IL-4R antagonist, or an IL-33 antagonist and an IL-4R antagonist to a subject in need thereof can cause a decrease in average number of nighttime awakenings from baseline of about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 times per night, 2.0 times per night, or more at week 12.

### 22-Item Sinonasal Outcome Test (SNOT-22) Score

According to certain embodiments, administration or use of an IL-33 antagonist, an IL-4R antagonist, or an IL-33 antagonist and an IL-4R antagonist to a patient results in a decrease from baseline of 22-item Sinonasal Outcome Test (SNOT-22). The SNOT-22 is a validated questionnaire to assess the impact of chronic rhinosinusitis on quality of life (Hopkins et al 2009, Clin. Otolaryngol. 34: 447-454).

According to certain exemplary embodiments, therapeutic methods or uses that result in a decrease in SNOT-22 score from baseline of at least 1 point at week 12 following initiation of treatment with a pharmaceutical composition comprising an IL-33 antagonist, an IL-4R antagonist, or an IL-33 antagonist and an IL-4R antagonist, are provided. For example, administration or use of an IL-33 antagonist, an IL-4R antagonist, or an IL-33 antagonist and an IL-4R antagonist to a subject in need thereof can cause a decrease in SNOT-22 score from baseline of about 1, 2, 3, 4, 5, 6, 7, 8, 9, 10, 11, 12, 13 points, or more at week 12.

### Biomarkers

According to certain embodiments, administration or use of an IL-33 antagonist, an IL-4R antagonist, or an IL-33 antagonist and an IL-4R antagonist to a patient results in an improvement in lung function as measured by a biomarker. For example, the biomarker may be fractional exhaled nitric oxide (FeNO), eotaxin-3, total IgE, periostin, or thymus and activation-regulated chemokine (TARC). In certain embodiments, an improvement in lung function is indicated by a reduction or increase (as appropriate) at week 4, week 12 or week 24 following treatment.

### Methods for Treating Asthma

In some embodiments, the invention provides methods for treating allergic asthma, including, e.g., mild allergic asthma and mild persistent allergic asthma, in a subject in need thereof, wherein the methods comprise administering a pharmaceutical composition comprising an IL-33 antagonist, an IL-4R antagonist, or an IL-33 antagonist and an IL-4R antagonist to the subject. Also provided is a pharmaceutical composition comprising an anti-IL-33 antagonist, an IL-4R antagonist, or an IL-33 antagonist and an IL-4R antagonist to treat allergic asthma, including, e.g., mild allergic asthma and mild persistent allergic asthma, in a subject in need thereof. In certain embodiments, the methods are useful for treating allergic asthma in a subject. In some embodiments, the methods are useful for treating mild persistent allergic asthma in a subject.

As used herein, the term “asthma” can be used interchangeably with “intermittent asthma,” or “bronchial asthma.” “Asthma,” “bronchial asthma” and “intermittent asthma” refer to asthma in which one or any combination of the following are true: symptoms occur 2 or fewer days per week; symptoms do not interfere with normal activities; nighttime symptoms occur fewer than 2 days per month; or one or more lung function tests (e.g., forced expiratory volume in one second (FEV₁) and/or peak expiratory flow (PEF) of greater than 80%) are normal when the subject is not suffering from an asthma attack.

“Allergic asthma” refers to asthma that is triggered by allergens, e.g., inhaled allergens (e.g., perennial aeroallergens and seasonal aeroallergens), such as dust mites, pet dander, pollen, fungi and the like. In certain embodiments, the allergen is a house dust mite (HDM) allergen (e.g., a perennial aeroallergen).

As used herein, a “perennial aeroallergen” refers to airborne allergens that can be present in the environment year-round, such as dust mites, fungi, dander and the like. Perennial aeroallergens include, but are not limited to, Alternaria alternata, Aspergillus fumigatus, Aureobasidium pullulans, Candida albicans, Cladosporium herbarum, Dermatofagoides farinae, Dermatofagoides pteronyssinus, Mucor racemosus, Penicillium chrysogenum, Phoma betae, Setomelanomma rostrata, Stemphylium herbarum, cat dander, dog dander, cow dander, chicken feathers, goose feathers, duck feathers, cockroach (e.g., German cockroach, Oriental cockroach), mouse urine, peanut dust, tree nut dust, and the like.

As used herein, a “seasonal aeroallergen” refers to airborne allergens that are present in the environment seasonally, such as pollens and spores. Seasonal aeroallergens include, but are not limited to, tree pollen (e.g., birch, alder, cedar, hazel, hornbeam, horse chestnut, willow, poplar, linden, pine, maple, oak, olive and the like), grass pollen (e.g., ryegrass, cat’s tail and the like), weed pollen (e.g., ragweed, plantain, nettles, mugwort, fat hen, sorrel and the like), fungal spores that increase during particular seasons, temperatures, etc. (e.g., molds), and the like.

As used herein, the term “persistent asthma” or “persistent bronchial asthma” refers to asthma that is more severe than (bronchial) asthma/intermittent (bronchial) asthma. A subject suffering from persistent asthma or persistent bronchial asthma experiences one or more of the following: symptoms more than 2 days per week; symptoms that interfere with normal activities; nighttime symptoms that occur more than 2 days per month; or one or more lung function tests (e.g., forced expiratory volume in one second (FEV₁) and/or peak expiratory flow (PEF) of less than 80%) that are not normal when the subject is not suffering from an asthma attack; the subject relies on daily asthma control medication; the subject has taken a systemic steroid more than once in the last year after a severe asthma flare-up; or use of a short-acting beta-2 agonist more than two days per week for relief of asthma symptoms.

Asthma/intermittent asthma, bronchial asthma/intermittent bronchial asthma, and persistent asthma/persistent bronchial asthma can be categorized as “mild,” “moderate,” “severe” or “moderate-to-severe.” “Mild intermittent asthma” or “mild intermittent bronchial asthma” is defined as having symptoms less than once a week, and having forced expiratory volume in one second (FEV₁) or peak expiratory flow (PEF) ≥80%. “Mild persistent asthma” or “mild persistent bronchial asthma” differs in that symptoms frequency is greater than once per week but less than once per day, and variability in FEV₁ or PEF is <20%-30%. “Moderate intermittent asthma” or “moderate intermittent bronchial asthma” is defined as having symptoms less than once a week, and having forced expiratory volume in one second (FEV₁) or peak expiratory flow (PEF) of 60-80%. “Moderate persistent asthma” or “moderate persistent bronchial asthma” is defined as having daily symptoms, exacerbations that may affect activity and/or sleep, nocturnal symptoms more than once a week, daily use of inhaled short-acting beta-2 agonist and having forced expiratory volume in one second (FEV₁) or peak expiratory flow (PEF) of 60-80%. “Severe intermittent asthma” or “severe intermittent bronchial asthma” is defined as having symptoms less than once a week, and having forced expiratory volume in one second (FEV₁) or peak expiratory flow (PEF) of 60%. “Severe persistent asthma” or “severe persistent bronchial asthma” is defined as having daily symptoms, frequent exacerbations that may affect activity and/or sleep, frequent nocturnal symptoms, limitation of physical activities, daily use of inhaled short-acting beta-2 agonist, and having forced expiratory volume in one second (FEV₁) or peak expiratory flow (PEF) of 60%. “Moderate-to-severe intermittent asthma” or “moderate-to-severe intermittent bronchial asthma” is defined as having symptoms between those of moderate intermittent asthma/moderate intermittent bronchial asthma and severe intermittent asthma/severe intermittent bronchial asthma. “Moderate-to-severe persistent asthma” or “moderate-to-severe persistent bronchial asthma” is defined as having symptoms between those of moderate persistent asthma/moderate persistent bronchial asthma and severe persistent asthma/severe persistent bronchial asthma.

As used herein, the term “inadequately controlled asthma” refers to patients whose asthma is either “not well controlled” or “very poorly controlled” as defined by the “Expert Panel Report 3: Guidelines for the Diagnosis and Management of Asthma,” National Heart, Blood and Lung Institute, NIH, Aug. 28, 2007. “Not well controlled asthma” is defined as having symptoms greater than two days per week, nighttime awakenings one to three times per week, some limitations on normal activity, short-acting beta₂-agonist use for symptom control greater than two days per week, FEV₁ of 60-80% of predicted and/or personal best, an ATAQ score of 1-2, an ACQ score of 1.5 or greater, and an ACT score of 16-19. “Very poorly controlled asthma” is defined as having symptoms throughout the day, nighttime awakenings four times or more per week, extreme limitations on normal activity, short-acting beta₂-agonist use for symptom control several times per day, FEV₁ of less than 60% of predicted and/or personal best, an ATAQ score of 3-4, an ACQ score of N/A, and an ACT score of less than or equal to 15.

In some embodiments, a subject is identified as having “moderate-to-severe uncontrolled” asthma if the subject receives such a diagnosis from a physician, based on the Global Initiative for Asthma (GINA) 2009 Guidelines, and one or more of the following criteria: i) Existing treatment with moderate-to-high dose ICS/LABA (2 fluticasone propionate 250 µg twice daily or equipotent ICS daily dosage) with a stable dose of ICS/LABA for greater than or equal to 1 month prior to administration of an initial dose of IL-4R antagonist, IL-33 antagonist, or an initial dose of IL-33 antagonist and IL-4R antagonist; ii) FEV₁ 40 to 80% predicted normal prior to administration of an initial dose of IL-4R antagonist, IL-33 antagonist, or an initial dose of IL-33 antagonist and IL-4R antagonist; iii) ACQ-5 score greater than or equal to 1.5 prior to administration of an initial dose of IL-4R antagonist, IL-33 antagonist, or an initial dose of IL-33 antagonist and IL-4R antagonist; iv) reversibility of at least 12% and 200 mL in FEV₁ after 200 µg to 400 µg (2 to 4 inhalations) of salbutamol/albuterol prior to administration of an initial dose of IL-4R antagonist, IL-33 antagonist, or an initial dose of IL-33 antagonist and IL-4R antagonist; or v) has experienced, within 1 year prior to administration of an initial dose of IL-4R antagonist, IL-33 antagonist, or an initial dose of IL-33 antagonist and IL-4R antagonist, any of the following events: (a) treatment with greater than or equal to 1 systemic (oral or parenteral) steroid burst for worsening asthma, (b) hospitalization or an emergency/urgent medical care visit for worsening asthma.

“Severe asthma” refers to asthma in which adequate control cannot be achieved by high-dose treatment with inhaled corticosteroids and additional controllers (e.g., long-acting inhaled beta 2 agonists, montelukast, and/or theophylline) or by oral corticosteroid treatment (e.g., for at least six months per year), or is lost when the treatment is reduced. In certain embodiments, severe asthma includes asthma that is treated with high-dose ICS and at least one additional controller (e.g., LABA, montelukast, or theophylline) or oral corticosteroids >6 months/year, wherein at least one of the following occurs or would occur if treatment is reduced: ACT <20 or ACQ >1.5; at least 2 exacerbations in the last 12 months; at least 1 exacerbation treated in hospital or requiring mechanical ventilation in the last 12 months; or FEV1 <80% (if FEV1/FVC below the lower limit of normal).

“Steroid-dependent asthma” refers to asthma which requires one or more of the following treatments: frequent, short term oral corticosteroid treatment bursts in the past 12 months; regular use of high dose inhaled corticosteroids in the past 12 months; regular use of injected long acting corticosteroids; daily use of oral corticosteroids; alternate-day oral corticosteroids; or prolonged use of oral corticosteroids in the past year.

“Oral corticosteroid-dependent asthma” refers to a subject having ≥3 30-day oral corticosteroid (OCS) fills over a 12-month period and a primary asthma diagnosis within 12 months of the first OCS fill. Subjects with OCS-dependent asthma may also experience one or any combination of the following: have received physician prescribed LABA and high dose ICS (total daily dose >500 µg fluticasone propionate dry powder formulation equivalent) for at least 3 months (the ICS and LABA can be parts of a combination product, or given by separate inhalers); have received additional maintenance asthma controller medications according to standard practice of care e.g., leukotriene receptor antagonists (LTRAs), theophylline, long-acting muscarinic antagonists (LAMAs), secondary ICS and cromones; received OCS for the treatment of asthma at a dose of between ≥ 7.5 to ≤ 30 mg (prednisone or prednisolone equivalent); have received an OCS dose administered every other day (or different doses every other day); morning pre-bronchodilator (BD) FEV1 of < 80% predicted normal; have evidence of asthma as documented by post-BD (albuterol/salbutatomol) reversibility of FEV1 ≥12% and ≥200 mL (15-30 min after administration of 4 puffs of albuterol/salbutamol); or have a history of at least one asthma exacerbation event within 12 months.

In one aspect, methods for treating asthma are provided comprising: (a) selecting a patient that exhibits a blood eosinophil level of at least 300 cells per microliter; and (b) administering to the patient a pharmaceutical composition comprising an IL-33 antagonist, an IL-4R antagonist, or an IL-33 antagonist and an IL-4R antagonist. In one aspect of the composition for use, the patient exhibits a blood eosinophil level of at least 300 cells per microliter.

In another aspect, methods for treating asthma are provided comprising: (a) selecting a patient that exhibits a blood eosinophil level of 150-299 cells per microliter; and (b) administering to the patient a pharmaceutical composition comprising an IL-33 antagonist, an IL-4R antagonist, or an IL-33 antagonist and an IL-4R antagonist. In one aspect of the composition for use, the patient exhibits a blood eosinophil level of 150-299 cells per microliter.

In another aspect, methods for treating asthma are provided comprising: (a) selecting a patient that exhibits a blood eosinophil level of less than 150 cells per microliter; and (b) administering to the patient a pharmaceutical composition comprising an IL-33 antagonist, an IL-4R antagonist, or an IL-33 antagonist and an IL-4R antagonist. In one aspect of the composition for use, the patient exhibits a blood eosinophil level of less than 150 cells per microliter.

In one aspect, methods for treating asthma are provided comprising: (a) selecting a patient that exhibits a low level of periostin level; and (b) administering to the patient a pharmaceutical composition comprising an IL-33 antagonist, an IL-4R antagonist, or an IL-33 antagonist and an IL-4R antagonist. In one aspect of the composition for use, the patient exhibits a low level of periostin level.

In another aspect, methods for treating asthma are provided comprising: (a) selecting a patient that exhibits a high level of periostin; and (b) administering to the patient a pharmaceutical composition comprising an IL-33 antagonist, an IL-4R antagonist, or an IL-33 antagonist and an IL-4R antagonist. In one aspect of the composition for use, the patient exhibits a high level of periostin level.

As used herein, a “high level of periostin” refers to a blood periostin measurement of greater than or equal to about 60 ng/mL, greater than or equal to about 65 ng/mL, greater than or equal to about 70 ng/mL, greater than or equal to about 75 ng/mL, or greater than or equal to about 80 ng/mL, greater than or equal to about 85 ng/mL, greater than or equal to about 90 ng/mL, greater than or equal to about 95 ng/mL, greater than or equal to about 100 ng/mL. In particularly exemplary embodiments, a high level of periostin is greater than or equal to about 75.0 ng/mL or greater than or equal to about 74.4 ng/mL.

As used herein, a “low level of periostin” refers to a blood periostin measurement of less than about 100 ng/mL, less than about 95 ng/mL, less than about 90 ng/mL, less than about 85 ng/mL, less than about 80 ng/mL, less than about 75 ng/mL, less than about 70 ng/mL, less than about 65 ng/mL, or less than about 60 ng/mL. In particularly exemplary embodiments, a low level of periostin is less than about 75.0 ng/mL or less than about 74.4 ng/mL.

In a related aspect, methods for treating asthma comprising an add-on therapy to background therapy are provided. In a related aspect, also provided is an IL-33 antagonist for use to treat allergic asthma in a patient, wherein IL-33 antagonist is used as an add-on therapy to background therapy. In certain embodiments, an IL-33 antagonist, an IL-4R antagonist, or an IL-33 antagonist and an IL-4R antagonist is administered as an add-on therapy to an asthma patient who is on background therapy for a certain period of time (e.g., 1 week, 2 weeks, 3 weeks, 1 month, 2 months, 5 months, 12 months, 18 months, 24 months, or longer) (also called the “stable phase”). In some embodiments, the background therapy comprises a ICS and/or a LABA.

In some embodiments, the invention includes a method for reducing an asthma patient’s dependence on ICS and/or LABA for the treatment of one or more allergic asthma exacerbations comprising: (a) selecting a patient who has asthma that is not well-controlled with a background asthma therapy comprising an ICS, a LABA, or a combination thereof; and administering to the patient a pharmaceutical composition comprising an IL-33 antagonist, an IL-4R antagonist, or an IL-33 antagonist and an IL-4R antagonist. In one aspect of the composition for use, the patient has asthma that is not well-controlled with a background asthma therapy comprising an ICS, a LABA, or a combination thereof.

In some embodiments, the invention encompasses methods to treat or alleviate conditions or complications associated with asthma, such as chronic rhinosinusitis, allergic rhinitis, allergic fungal rhinosinusitis, allergic broncho-pulmonary aspergillosis, unified airway disease, Churg-Strauss syndrome, vasculitis, chronic obstructive pulmonary disease (COPD), and exercise induced bronchospasm. Also provided is a pharmaceutical composition comprising an anti-IL-33 antagonist, an IL-4R antagonist, or an IL-33 antagonist and an IL-4R antagonist to treat conditions or complications associated with asthma, such as chronic rhinosinusitis, allergic rhinitis, allergic fungal rhinosinusitis, allergic broncho-pulmonary aspergillosis, unified airway disease, Churg-Strauss syndrome, vasculitis, chronic obstructive pulmonary disease (COPD), and exercise induced bronchospasm, in a subject in need thereof.

The invention also includes methods for treating persistent asthma. Also provided is a pharmaceutical composition comprising an anti-IL-33 antagonist, an IL-4R antagonist, or an IL-33 antagonist and an IL-4R antagonist to treat persistent asthma, in a subject in need thereof. As used herein, the term “persistent asthma” means that the subject has symptoms at least once a week at day and/or at night, with the symptoms lasting a few hours to a few days. In certain alternative embodiments, the persistent asthma is “mildly persistent” (e.g., more than twice a week but less than daily with symptoms severe enough to interfere with daily activities or sleep and/or where pulmonary function is normal or reversible with inhalation of a bronchodilator), “moderately persistent” (e.g., symptoms occurring daily with sleep interrupted at least weekly and/or with pulmonary function moderately abnormal), or “severely persistent” (e.g., continuous symptoms despite the correct use of approved medications and/or where pulmonary function is severely affected).

### Interleukin-33 (IL-33) Antagonists and Interleukin-4 Receptor (IL-4R) Antagonists

Methods disclosed herein optionally comprise administering to a subject in need thereof a therapeutic composition comprising an IL-33 antagonist. As used herein, an “IL-33 antagonist” is any agent that binds to or interacts with IL-33 and inhibits the normal biological signaling function of IL-33 when IL-33 is expressed on a cell in vitro or in vivo.

Methods disclosed herein optionally comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo.

Non-limiting examples of categories of IL-33 antagonists and IL-4R antagonists include small molecule IL-33 antagonists, small molecule IL-4R antagonists, anti-IL-33 aptamers, anti-IL-4R aptamers, peptide-based IL-33 antagonists or peptide-based IL-4R antagonists (e.g., “peptibody” molecules), and antibodies or antigen-binding fragments of antibodies that specifically bind human IL-33 or human IL-4R.

According to certain embodiments, the IL-33 antagonist comprises an anti-IL-33 antibody or antigen-binding fragment thereof that can be used in the context of the methods featured in the invention are described elsewhere herein. For example, in one embodiment, the IL-33 antagonist is an antibody or antigen-binding fragment thereof that specifically binds to an IL-33, and comprises the heavy chain and light chain (complementarity determining region) CDR sequences from the heavy chain variable region (HCVR) and light chain variable region (LCVR) of SEQ ID NOs: 2 and 10, respectively. In another embodiment, the IL-33 antagonist is an antibody or antigen-binding fragment thereof that specifically binds to an IL-33, and comprises the heavy chain and light chain CDR sequences of SEQ ID NOs: 4, 6 and 8, and SEQ ID NOs: 12, 14 and 16, respectively. In another embodiment, the IL-33 antagonist is an antibody or antigen-binding fragment thereof that specifically binds to an IL-33, and comprises an HCVR/LCVR pair of SEQ ID NOs: 2 and 10, respectively.

### REGN3500 HCVR, DNA Sequence:

aggtgcagct ggtggagtct gggggaaact tggaacagcc tggggggtcc cttagactct cctgtacagc ctctggattc
acctttagca gatctgccat gaactgggtc cgccgggctc cagggaaggg gctggagtgg gtctcaggaa ttagtggtag tggtggtcga
acatactacg cagactccgt gaagggccgg ttcaccatct ccagagacaa ttccaagaat acgctatatc tgcaaatgaa cagcctgagc
gccgaggaca cggccgcata ttactgtgcg aaagattcgt atactaccag ttggtacgga ggtatggacg tctggggcca
cgggaccacg gtcaccgtct cctca (SEQ ID NO: 1).

### REGN3500 HCVR, Amino Acid Sequence:

VQLVESGGNLEQPGGSLRLSCTASGFTFSRSAMNWVRRAPGKGLEWVSGI
SGSGGRTYYADSVKGRFTISRDNSKNTLYLQMNSLSAEDTAAYYCAKDSY
TTSWYGGMDVWGHGTTVTVSS (SEQ ID NO: 2).

### REGN3500 HCDR1, DNA Sequence:

ggattcacctt tagcagatct gcc (SEQ ID NO: 3).

### REGN3500 HCDR1, Amino Acid Sequence:

GFTFSRSA (SEQ ID NO: 4).

### REGN3500 HCDR2, DNA Sequence:

attagtggtag tggtggtcga aca (SEQ ID NO: 5).

### REGN3500 HCDR2, Amino Acid Sequence:

ISGSGGRT (SEQ ID NO: 6).

### REGN3500 HCDR3, DNA Sequence:

gcgaaagattc gtatactacc agttggtacg gaggtatgga cgtc (SEQ ID NO: 7).

### REGN3500 HCDR3, Amino Acid Sequence:

AKDSYTTSWYGGMDV (SEQ ID NO: 8).

### REGN3500 LCVR, DNA Sequence:

acatccagat gacccagtct ccatcttccg tgtctgcatc tgtaggagac agagtcacca tcacttgtcg ggcgagtcag
ggtattttca gctggttagc ctggtatcag cagaaaccag gaaaagcccc taagctcctg atctatgctg cttccagttt acaaagtggg
gtcccatcaa gattcagcgg cagtggatct gggacagatt tcactctcac catcagcagc ctgcagcctg aggattttgc aatttactat
tgtcaacagg ctaacagtgt cccgatcacc ttcggccaag ggacacgact ggagattaaa cga (SEQ ID NO: 9).

### REGN3500 LCVR, Amino Acid Sequence:

IQMTQSPSSVSASVGDRVTITCRASQGIFSWLAWYQQKPGKAPKLLIYAA
SSLQSGVPSRFSGSGSGTDFTLTISSLQPEDFAIYYCQQANSVPITFGQG
TRLEIKR (SEQ ID NO: 10).

### REGN3500 LCDR1, DNA Sequence:

cagggtatttt cagctgg (SEQ ID NO: 11).

### REGN3500 LCDR1, Amino Acid Sequence:

QGIFSW (SEQ ID NO: 12).

### REGN3500 LCDR2, DNA Sequence:

gctgcttcc (SEQ ID NO: 13).

### REGN3500 LCDR2, Amino Acid Sequence:

AAS (SEQ ID NO: 14).

### REGN3500 LCDR3, DNA Sequence:

caacaggctaa cagtgtcccg atcacc (SEQ ID NO: 15).

### REGN3500 LCDR3, Amino Acid Sequence:

QQANSVPIT (SEQ ID NO: 16).

In another embodiment, the IL-33 antagonist is the REGN3500 antibody, which comprises an HC/LC pair of SEQ ID NOs: 18 and 20, respectively.

### REGN3500 Heavy Chain DNA Sequence:

aggtgcagct ggtggagtct gggggaaact tggaacagcc tggggggtcc cttagactct cctgtacagc ctctggattc
acctttagca gatctgccat gaactgggtc cgccgggctc cagggaaggg gctggagtgg gtctcaggaa ttagtggtag tggtggtcga
acatactacg cagactccgt gaagggccgg ttcaccatct ccagagacaa ttccaagaat acgctatatc tgcaaatgaa cagcctgagc
gccgaggaca cggccgcata ttactgtgcg aaagattcgt atactaccag ttggtacgga ggtatggacg tctggggcca
cgggaccacg gtcaccgtct cctcagcctc caccaagggc ccatcggtct tccccctggc gccctgctcc aggagcacct
ccgagagcac agccgccctg ggctgcctgg tcaaggacta cttccccgaa ccggtgacgg tgtcgtggaa ctcaggcgcc
ctgaccagcg gcgtgcacac cttcccggct gtcctacagt cctcaggact ctactccctc agcagcgtgg tgaccgtgcc ctccagcagc
ttgggcacga agacctacac ctgcaacgta gatcacaagc ccagcaacac caaggtggac aagagagttg agtccaaata
tggtccccca tgcccaccct gcccagcacc tgagttcctg gggggaccat cagtcttcct gttcccccca aaacccaagg acactctcat
gatctcccgg acccctgagg tcacgtgcgt ggtggtggac gtgagccagg aagaccccga ggtccagttc aactggtacg
tggatggcgt ggaggtgcat aatgccaaga caaagccgcg ggaggagcag ttcaacagca cgtaccgtgt ggtcagcgtc
ctcaccgtcc tgcaccagga ctggctgaac ggcaaggagt acaagtgcaa ggtctccaac aaaggcctcc cgtcctccat
cgagaaaacc atctccaaag ccaaagggca gccccgagag ccacaggtgt acaccctgcc cccatcccag gaggagatga
ccaagaacca ggtcagcctg acctgcctgg tcaaaggctt ctaccccagc gacatcgccg tggagtggga gagcaatggg
cagccggaga acaactacaa gaccacgcct cccgtgctgg actccgacgg ctccttcttc ctctacagca ggctcaccgt
ggacaagagc aggtggcagg aggggaatgt cttctcatgc tccgtgatgc atgaggctct gcacaaccac tacacacaga agtccctctc
cctgtctctg ggtaaatga (SEQ ID NO: 17).

### REGN3500 Heavy Chain Amino Acid Sequence:

VQLVESGGNLEQPGGSLRLSCTASGFTFSRSAMNWVRRAPGKGLEWVSGI
SGSGGRTYYADSVKGRFTISRDNSKNTLYLQMNSLSAEDTAAYYCAKDSY
TTSWYGGMDVWGHGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK (
SEQ ID NO: 18).

### REGN3500 Light Chain DNA Sequence:

acatccagat gacccagtct ccatcttccg tgtctgcatc tgtaggagac agagtcacca tcacttgtcg ggcgagtcag
ggtattttca gctggttagc ctggtatcag cagaaaccag gaaaagcccc taagctcctg atctatgctg cttccagttt acaaagtggg
gtcccatcaa gattcagcgg cagtggatct gggacagatt tcactctcac catcagcagc ctgcagcctg aggattttge aatttactat
tgtcaacagg ctaacagtgt cccgatcacc ttcggccaag ggacacgact ggagattaaa cgaactgtgg ctgcaccatc tgtcttcatc
ttcccgccat ctgatgagca gttgaaatct ggaactgcct ctgttgtgtg cctgctgaat aacttctatc ccagagaggc caaagtacag
tggaaggtgg ataacgccct ccaatcgggt aactcccagg agagtgtcac agagcaggac agcaaggaca gcacctacag
cctcagcagc accctgacgc tgagcaaagc agactacgag aaacacaaag tctacgcctg cgaagtcacc catcagggcc
tgagctcgcc cgtcacaaag agcttcaaca ggggagagtg ttag (SEQ ID NO: 19).

### REGN3500 Light Chain Amino Acid Sequence:

IQMTQSPSSVSASVGDRVTITCRASQGIFSWLAWYQQKPGKAPKLLIYAA
SSLQSGVPSRFSGSGSGTDFTLTISSLQPEDFAIYYCQQANSVPITFGQG
TRLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC (SEQ ID NO: 20).

According to certain embodiments, the IL-4R antagonist comprises an anti-IL-4R antibody or antigen-binding fragment thereof that can be used in the context of the methods featured in the invention are described elsewhere herein. For example, in one embodiment, the IL-4R antagonist is an antibody or antigen-binding fragment thereof that specifically binds to an IL-4R, and comprises the heavy chain and light chain (complementarity determining region) CDR sequences from the heavy chain variable region (HCVR) and light chain variable region (LCVR) of SEQ ID NOs: 27 and 28, respectively. In another embodiment, the IL-4R antagonist is an antibody or antigen-binding fragment thereof that specifically binds to an IL-4R, and comprises the heavy chain and light chain CDR sequences of SEQ ID NOs: 21, 22 and 23, and SEQ ID NOs: 24, 25 and 26, respectively. In another embodiment, the IL-4R antagonist is an antibody or antigen-binding fragment thereof that specifically binds to an IL-4R, and comprises an HCVR/LCVR pair of SEQ ID NOs: 27 and 28, respectively.

### Dupilumab HCDR1 Amino Acid Sequence:

GFTFRDYA (SEQ ID NO: 21).

### Dupilumab HCDR2 Amino Acid Sequence:

ISGSGGNT (SEQ ID NO: 22).

### Dupilumab HCDR3 Amino Acid Sequence:

AKDRLSITIRPRYYGL (SEQ ID NO: 23).

### Dupilumab LCDR1 Amino Acid Sequence:

QSLLYSIGYNY (SEQ ID NO: 24).

### Dupilumab LCDR2 Amino Acid Sequence:

LGS (SEQ ID NO: 25).

### Dupilumab LCDR3 Amino Acid Sequence:

MQALQTPYT (SEQ ID NO: 26).

### Dupilumab HCVR Amino Acid Sequence:

EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVS (SEQ ID NO: 27).

### Dupilumab LCVR Amino Acid Sequence:

DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIK (SEQ IDNO: 28).

In another embodiment, the IL-4R antagonist is dupilumab.

### Dupilumab HC Amino Acid Sequence:

EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
G (SEQ ID NO: 29) (amino acids 1-124 = HCVR;amino
acids 125-451 = HC constant).

### Dupilumab LC Amino Acid Sequence:

DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (SEQ ID NO: 30) (amino acids 1
-112 = LCVR; amino acids 112-219 = LC constant).

The term “human IL-33” (hIL-33) refers to a human cytokine receptor that specifically binds to interleukin-33 (IL-33). The term “human IL-4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-33 antibody, the anti-IL-4R antibody, or an antigen-binding portion thereof may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include, but are not limited to: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies featured in the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The invention encompasses antibodies having one or more mutations in the hinge, C_{H}2, or C_{H}3 region, which may be desirable, for example, in production, to improve the yield of the desired antibody form.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-33 or IL-4R, as featured in the invention, includes antibodies that bind IL-33 or IL-4R, respectively, or portion thereof, with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-33 or human IL-4R may, however, have cross-reactivity to other antigens, such as IL-33 or IL-4R molecules from other (non-human) species.

The anti-IL-33 and anti-IL-4R antibodies useful for the methods may comprise one or more amino acid substitutions, insertions, and/or deletions (e.g. 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The invention includes methods involving the use of antibodies, and antigen-binding fragments thereof, that are derived from any of the amino acid sequences disclosed herein, wherein one or more amino acids (e.g. 1, 2, 3, 4, 5, 6, 7, 8, 9, or 10 amino acids) within one or more framework and/or one or more (e.g. 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the invention.

The invention also includes methods involving the use of anti-IL33 or anti-IL-4R antibodies comprising variants of any of the HCVR, LCVR, and/or CDR amino acid sequences disclosed herein having one or more conservative substitutions. For example, the invention includes the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

### Preparation of Human Antibodies

Methods for generating human antibodies in transgenic mice are known in the art. Any such known methods can be used to make human antibodies that specifically bind to human IL-33 or human IL-4R.

Using VELOCIMMUNE® technology (see, for example, US 6,596,541, Regeneron Pharmaceuticals) or any other known method for generating monoclonal antibodies, high affinity chimeric antibodies to IL-33 or IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody featured in the invention, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully human antibodies featured in the invention. While the constant region selected may vary according to specific use, high affinity antigen-binding and target specificity characteristics reside in the variable region.

In one embodiment, human antibody or antigen-binding fragment thereof that specifically binds IL-33 that can be used in the context of the methods featured in the invention comprises the three heavy chain CDRs (HCDR1, HCDR2 and HCDR3) contained within a heavy chain variable region (HCVR) having an amino acid sequence of SEQ ID NO: 2. The antibody or antigen-binding fragment may comprise the three light chain CDRs (LCVR1, LCVR2, LCVR3) contained within a light chain variable region (LCVR) having an amino acid sequence of SEQ ID NO: 10. In another embodiment, human antibody or antigen-binding fragment thereof that specifically binds IL-4R that can be used in the context of the methods featured in the invention comprises the three heavy chain CDRs (HCDR1, HCDR2 and HCDR3) contained within a heavy chain variable region (HCVR) having an amino acid sequence of SEQ ID NO: 27. The antibody or antigen-binding fragment may comprise the three light chain CDRs (LCVR1, LCVR2, LCVR3) contained within a light chain variable region (LCVR) having an amino acid sequence of SEQ ID NO: 28.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 2 and 10.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 4/6/8/12/14/16.

In certain embodiments, the antibody or antigen-binding fragment thereof comprises HCVR/LCVR amino acid sequence pairs of SEQ ID NOs: 2 and 10.

In one embodiment, the antibody is REGN3500, which comprises the HCVR/LCVR amino acid sequence pairs of SEQ ID NOs: 2 and 10, and comprises the heavy chain/light chain amino acid sequences pair of SEQ ID NOs: 18 and 20.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 27 and 28.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 21/22/23/24/25/26.

In certain embodiments, the antibody or antigen-binding fragment thereof comprises HCVR/LCVR amino acid sequence pairs of SEQ ID NOs: 27 and 28.

In one embodiment, the antibody is dupilumab, which comprises the HCVR/LCVR amino acid sequence pairs of SEQ ID NOs: 27 and 28, and comprises the heavy chain/light chain amino acid sequences pair of SEQ ID NOs: 29 and 30.

### Pharmaceutical Compositions

The invention includes methods that comprise administering an IL-33 antagonist, an IL-4R antagonist, or an IL-33 antagonist and an IL-4R antagonist, to a patient, wherein the IL-33 antagonist, the IL-4R antagonist, or the IL-33 antagonist and the IL-4R antagonist are contained within a pharmaceutical composition. The invention also includes an IL-33 antagonist, an IL-4R antagonist, or an IL-33 antagonist and an IL-4R antagonist for use, wherein the IL-33 antagonist, the IL-4R antagonist, or the IL-33 antagonist and the IL-4R antagonist, are contained within a pharmaceutical composition. The pharmaceutical compositions featured in the invention are formulated with suitable carriers, excipients, and other agents that provide suitable transfer, delivery, tolerance, and the like. A multitude of appropriate formulations can be found in the formulary known to all pharmaceutical chemists: Remington’s Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J. Pharm. Sci. Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-33 antibodies or anti-IL-4R antibodies may be determined empirically. For example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions featured in the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition featured in the invention can be delivered subcutaneously or intravenously with a standard needle and syringe. In addition, with respect to subcutaneous delivery, a pen delivery device (e.g., an autoinjector pen) readily has applications in delivering a pharmaceutical composition featured in the invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition featured in the invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions featured in the invention may be administered using, e.g., a microcatheter (e.g., an endoscope and microcatheter), an aerosolizer, a powder dispenser, a nebulizer or an inhaler. The methods include administration of an IL-33 antagonist or an IL-4R antagonist to a subject in need thereof, in an aerosolized formulation. For example, aerosolized antibodies to IL-33 or IL-4R may be administered to treat asthma in a patient. Aerosolized antibodies can be prepared as described in, for example, US 8,178 098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition’s target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the invention are disclosed, e.g., in U.S. Pat. Application Publication No. 2012/0097565.

### Dosage

The amount of IL-33 antagonist (e.g., an anti-IL-33 antibody or antigen-binding fragment thereof) or IL-4R antagonist (e.g., anti-IL-4R antibody or antigen-binding fragment thereof) administered to a subject according to the methods or uses featured in the invention is, generally, a therapeutically effective amount. As used herein, the phrase “therapeutically effective amount” means an amount of IL-33 antagonist or IL-4R antagonist that results in one or more of: (a) a reduction in the incidence of allergic asthma exacerbations; (b) an improvement in one or more allergic asthma-associated parameters (as defined elsewhere herein); and/or (c) a detectable improvement in one or more symptoms or indicia of an upper airway inflammatory condition. A “therapeutically effective amount” also includes an amount of IL-33 antagonist or IL-4R antagonist that inhibits, prevents, lessens, or delays the progression of allergic asthma in a subject.

In the case of an anti-IL-33 antibody or an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-33 antibody or anti-IL-4R antibody. In certain embodiments, 300 mg of an anti-IL-4R antibody is administered.

The amount of IL-33 antagonist or IL-4R antagonist contained within the individual doses may be expressed in terms of milligrams of antibody per kilogram of patient body weight (i.e., mg/kg). For example, the IL-4R antagonist may be administered to a patient at a dose of about 0.0001 to about 30 mg/kg of patient body weight. In the case of an anti-IL-33 antibody or an anti-IL-4R antibody, a therapeutically effective amount can be about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of the anti-IL-33 antibody or anti-IL-4R antibody. In certain embodiments, 10 mg/kg of an anti-IL-33 antibody is administered.

In some embodiments, the dose of IL-4R antagonist or IL-33 antagonist may vary according to eosinophil count. For example, the subject may have a blood eosinophil count (high blood eosinophils) ≥300 cells/µL, or 300 - 499 cells/µL or ≥500 cells/µL (HEos); a blood eosinophil count of 200 to 299 cells/µL (moderate blood eosinophils); or a blood eosinophil count <200 cells/µL (low blood eosinophils).

In some embodiments, the dose of IL-4R antagonist or IL-33 antagonist may vary according to periostin levels. For example, the subject may have high periostin levels (e.g., ≥75.0 ng/mL or 74.4 ng/mL) or low periostin levels (e.g., <75.0 ng/mL or <74.4 ng/mL).

In some embodiments, the methods comprise an initial dose of about 5 mg/kg to about 15 mg/kg of an IL-33 antagonist, e.g., about 10 mg/kg of an IL-33 antagonist. In certain embodiments, the methods comprise an initial dose of about 200 to about 600 mg of an IL-4R antagonist, e.g., about 600 mg of an IL-4R antagonist.

In certain embodiments, the methods comprise one or more maintenance doses of about 200 to about 300 mg of the IL-4R antagonist.

In certain embodiments, ICS and LABA are administered for the duration of administration of the IL-33 antagonist. In certain embodiments, ICS and LABA are administered for the duration of administration of the IL-4R antagonist.

In certain embodiments, the initial dose comprises 600 mg of an anti-IL-4R antibody or antigen-binding fragment thereof, and the one or more maintenance doses comprises 300 mg of the antibody or antigen-binding fragment thereof administered every other week.

In other embodiments, the initial dose comprises 600 mg of an anti-IL-4R antibody or antigen-binding fragment thereof, and the one or more maintenance doses comprises 300 mg of the antibody or antigen-binding fragment thereof administered every fourth week.

In other embodiments, the initial dose comprises 600 mg of an anti-IL-4R antibody or antigen-binding fragment thereof, and the one or more maintenance doses comprises 300 mg of the antibody or antigen-binding fragment thereof administered once a week.

In other embodiments, the initial dose comprises 600 mg of an anti-IL-4R antibody or antigen-binding fragment thereof, and the one or more maintenance doses comprises 300 mg of the antibody or antigen-binding fragment thereof administered every third week.

In one embodiment, the subject is 6 to <18 years old and the IL-33 antibody or antigen-binding fragment thereof or the IL-4R antibody or antigen binding fragment thereof is administered at 2 mg/kg or 4 mg/kg.

In another embodiment, the subject is 12 to <18 years old and the IL-33 antibody or antigen-binding fragment thereof or the IL-4R antibody or antigen binding fragment thereof is administered at 2 mg/kg or 4 mg/kg.

In another embodiment, the subject is 6 to <12 years old and the IL-33 antibody or antigen-binding fragment thereof or the IL-4R antibody or antigen binding fragment thereof is administered at 2 mg/kg or 4 mg/kg.

In another embodiment, the subject is 2 to <6 years old and the IL-33 antibody or antigen-binding fragment thereof or the IL-4R antibody or antigen binding fragment thereof is administered at 2 mg/kg or 4 mg/kg.

In yet another embodiment, the subject is <2 years old and the IL-33 antibody or antigen-binding fragment thereof or the IL-4R antibody or antigen binding fragment thereof is administered at 2 mg/kg or 4 mg/kg.

### Combination Therapies

Certain embodiments of the methods featured in the invention comprise administering to the subject one or more additional therapeutic agents in combination with the IL-33 antagonist, one or more additional therapeutic agents in combination with the IL-4R antagonist, or one or more additional therapeutic agents in combination with the IL-33 antagonist and the IL-4R antagonist. Certain embodiments of the invention comprise the IL-33 antagonist, the IL-4R antagonist or the IL-33 antagonist and the IL-4R antagonist for use in combination with additional therapeutic agents. Certain embodiments of the invention comprise a combination of the IL-33 antagonist, the IL-4R antagonist or the IL-33 antagonist and the IL-4R antagonist with additional therapeutic agents for use. As used herein, the expression “in combination with” means that the additional therapeutic agents are administered before, after, or concurrent with the pharmaceutical composition comprising the IL-4R antagonist, IL-33 antagonist, or the IL-33 antagonist and the IL-4R antagonist. In some embodiments, the term “in combination with” includes sequential or concomitant administration of an IL-33 antagonist, an IL-4R antagonist, or an IL-33 antagonist and an IL-4R antagonist, and an additional therapeutic agent. The invention includes methods to treat asthma or an associated condition or complication or to reduce at least one exacerbation, comprising administration of an IL-33 antagonist, an IL-4R antagonist, or an IL-33 antagonist and an IL-4R antagonist, in combination with an additional therapeutic agent for additive or synergistic activity.

For example, when administered “before” the pharmaceutical composition comprising an IL-33 antagonist, an IL-4R antagonist, or an IL-33 antagonist and an IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-33 antagonist, the IL-4R antagonist, or the IL-33 antagonist and the IL-4R antagonist. When administered “after” the pharmaceutical composition comprising an IL-33 antagonist, an IL-4R antagonist, or an IL-33 antagonist and an IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-33 antagonist, the IL-4R antagonist, or the IL-33 antagonist and the IL-4R antagonist. Administration “concurrent” with the pharmaceutical composition comprising an IL-33 antagonist, an IL-4R antagonist, or an IL-33 antagonist and an IL-4R antagonist, means that the additional therapeutic agent is administered to the subject in a separate dosage form within less than 5 minutes (before, after, or at the same time) of administration of the pharmaceutical composition comprising the IL-33 antagonist, the IL-4R antagonist, or the IL-33 antagonist and the IL-4R antagonist, or administered to the subject as a single combined dosage formulation comprising both the additional therapeutic agent and the IL-33 antagonist, the IL-4R antagonist, or the IL-33 antagonist and the IL-4R antagonist.

The additional therapeutic agent may be, e.g., another IL-33 antagonist, another IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a short-acting beta₂ agonist (e.g., bitolterol, fenoterol, isoprenaline, isoprotenerol, levosalbutamol, levalbuterol, orciprenaline, metaproterenol, pirbuterol, procaterol, ritodrine, salbutamol, albuterol or terbutaline), a long-acting beta₂ agonist (e.g., salmeterol or formoterol), an inhaled corticosteroid (e.g., fluticasone or budesonide), a systemic corticosteroid (e.g., oral or intravenous), methylxanthine, nedocromil sodium, cromolyn sodium, or combinations thereof. For example, in certain embodiments, the pharmaceutical composition comprising an IL-4R antagonist, an IL-33 antagonist, or an IL-33 antagonist and an IL-4R antagonist, is administered with a combination comprising a long-acting beta₂ agonist and an inhaled corticosteroid (e.g., fluticasone + salmeterol [e.g., Advair® (GlaxoSmithKline)]; or budesonide + formoterol [e.g., SYMBICORT® (Astra Zeneca)]).

### Administration Regimens

According to certain embodiments, multiple doses of an IL-33 antagonist, an IL-4R antagonist, or an IL-33 antagonist and an IL-4R antagonist, may be administered to a subject over a defined time course. Such methods or uses comprise sequentially administering to a subject multiple doses of an IL-33 antagonist, an IL-4R antagonist, or an IL-33 antagonist and an IL-4R antagonist. As used herein, “sequentially administering” means that each dose of an IL-33 antagonist, an IL-4R antagonist, or an IL-33 antagonist and an IL-4R antagonist, is administered to the subject at a different point in time, e.g., on different days separated by a predetermined interval (e.g., hours, days, weeks, or months). Included are methods or uses that comprise sequentially administering to the patient a single initial dose of an IL-33 antagonist, an IL-4R antagonist, or an IL-33 antagonist and an IL-4R antagonist, followed by one or more secondary doses of the IL-33 antagonist, the IL-4R antagonist, or the IL-33 antagonist and the IL-4R antagonist, and optionally followed by one or more tertiary doses of the IL-33 antagonist, the IL-4R antagonist, or the IL-33 antagonist and the IL-4R antagonist.

The invention includes methods or uses comprising administering to a subject a pharmaceutical composition comprising an IL-33 antagonist, an IL-4R antagonist, or an IL-33 antagonist and an IL-4R antagonist at a dosing frequency of about four times a week, twice a week, once a week (q1w), once every two weeks (bi-weekly or q2w), once every three weeks (tri-weekly or q3w), once every four weeks (monthly or q4w), once every five weeks (q5w), once every six weeks (q6w), once every eight weeks (q8w), once every twelve weeks (q12w), or less frequently so long as a therapeutic response is achieved. In certain embodiments involving the administration of a pharmaceutical composition comprising an anti-IL-33 antibody or an anti-IL-4R antibody, once a week dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every two weeks dosing (bi-weekly dosing) of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every three weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every four weeks dosing (monthly dosing) of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every five weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every six weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every eight weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every twelve weeks dosing of an amount of about 75 mg, 100 mg, 150 mg, 200 mg, or 300 mg, can be employed. In one embodiment, the route of administration is subcutaneous.

The term “week” or “weeks” refers to a period of (n × 7 days) ±2 days, e.g. (n × 7 days) ±1 day, or (n × 7 days), wherein “n” designates the number of weeks, e.g. 1, 2, 3, 4, 5, 6, 8, 12 or more.

The terms “initial dose,” “subsequent dose(s),” “secondary dose(s),” and “tertiary dose(s),” refer to the temporal sequence of administration of the IL-4R antagonist or the IL-33 antagonist. Thus, the “initial dose” is the dose that is administered at the beginning of the treatment regimen (also referred to as the “baseline dose”); the “subsequent doses” or “secondary doses” are the doses that are administered after the initial dose; and the “tertiary doses” are the doses that are administered after the secondary doses. The initial, subsequent, secondary, and tertiary doses may all contain the same amount of IL-33 antagonist or IL-4R antagonist, but generally may differ from one another in terms of frequency of administration. In certain embodiments, however, the amount of IL-33 antagonist or IL-4R antagonist contained in the initial, subsequent, secondary and/or tertiary doses varies from one another (e.g., adjusted up or down as appropriate) during the course of treatment. In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg.

In certain embodiments, the initial dose is about 400 mg to about 600 mg of the IL-4R antagonist. In one embodiment, the initial dose is 400 mg of the IL-4R antagonist. In another embodiment, the initial dose is 600 mg of the IL-4R antagonist.

In certain embodiments, the subsequent dose is about 200 to about 300 mg of the IL-4R antagonist. In one embodiment, the subsequent dose is 200 mg of the IL-4R antagonist. In another embodiment, the subsequent dose is 300 mg of the IL-4R antagonist.

In certain embodiments, the initial dose is about 5 mg/kg to about 15 mg/kg of the IL-33 antagonist. In one embodiment, the initial dose is 10 mg/kg of the IL-33 antagonist.

In certain embodiments, the subsequent dose is about 5 mg/kg to about 15 mg/kg of the IL-33 antagonist. In one embodiment, the subsequent dose is 5 mg/kg of the IL-33 antagonist. In another embodiment, the subsequent dose is 10 mg/kg of the IL-33 antagonist. In another embodiment, no subsequent dose is administered to the subject (e.g., only an initial dose is administered to the subject).

In certain embodiments, the loading dose is two times the maintenance dose. In certain embodiments, the initial dose is the same amount as the maintenance dose. In certain embodiments, the initial dose is the only dose administered.

In some embodiments, the initial dose comprises 300 mg of the antibody or antigen-binding fragment thereof, and the one or more maintenance doses comprises 300 mg of the antibody or antigen-binding fragment thereof administered every other week.

In some embodiments, the initial dose comprises 300 mg of the antibody or antigen-binding fragment thereof, and the one or more maintenance doses comprises 300 mg of the antibody or antigen-binding fragment thereof administered every fourth week.

In some embodiments, a subject has mild allergic asthma, and the initial dose comprises 600 mg of the antibody or antigen-binding fragment thereof, and the one or more maintenance doses comprises 300 mg of the antibody or antigen-binding fragment thereof administered every fourth week.

In some embodiments, the initial dose comprises 10 mg/kg of the antibody or antigen-binding fragment thereof, and the one or more maintenance doses comprises 10 mg/kg of the antibody or antigen-binding fragment thereof administered every other week.

In some embodiments, the initial dose comprises 10 mg/kg of the antibody or antigen-binding fragment thereof, and the one or more maintenance doses comprises 10 mg/kg of the antibody or antigen-binding fragment thereof administered every fourth week.

In some embodiments, a subject has mild allergic asthma, and the initial dose comprises 10 mg/kg of the antibody or antigen-binding fragment thereof.

In one exemplary embodiment, each subsequent, secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-33 antagonist or IL-4R antagonist that is administered to a patient prior to the administration of the very next dose in the sequence with no intervening doses.

The methods or uses may include administering to a patient any number of secondary and/or tertiary doses of an IL-33 antagonist or an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The invention includes methods comprising sequential administration of an IL-33 antagonist, an IL-4R antagonist, or an IL-33 antagonist and an IL-4R antagonist and an additional therapeutic agent, to a patient to treat asthma or an associated condition. The invention also includes an IL-33 antagonist, an IL-4R antagonist, or an IL-33 antagonist and an IL-4R antagonist for use to a patient to treat asthma or an associated condition, wherein said patient is treated with sequential administration of an IL-33 antagonist or an IL-33 antagonist and an IL-4R antagonist and an additional therapeutic agent. In some embodiments, the methods or uses comprise administering one or more doses of an IL-33 antagonist or one or more doses of both an IL-33 antagonist and an IL-4R antagonist followed by one or more doses (e.g., 2, 3, 4, 5, 6, 7, 8, or more) of an additional therapeutic agent. For example, one or more doses of about 75 mg to about 300 mg of an IL-4R antagonist and/or one or more doses of about 5 mg/kg to about 20 mg/kg of an IL-33 antagonist may be administered after which one or more doses (e.g., 2, 3, 4, 5, 6, 7, 8, or more) of an additional therapeutic agent (e.g., an inhaled corticosteroid or a beta2-agonist or any other therapeutic agent, as described elsewhere herein) may be administered to treat, alleviate, reduce or ameliorate one or more symptoms of asthma. In some embodiments, an IL-33 antagonist and/or an IL-33 antagonist are administered at one or more doses (e.g., 2, 3, 4, 5, 6, 7, 8, or more) resulting in an improvement in one or more asthma-associated parameters followed by the administration of a second therapeutic agent to prevent recurrence of at least one symptom of asthma. Alternative embodiments pertain to concomitant administration of an IL-33 antagonist and/or an IL-4R antagonist, and an additional therapeutic agent. For example, one or more doses (e.g., 2, 3, 4, 5, 6, 7, 8, or more) of an IL-33 antagonist and/or an IL-4R antagonist are administered and an additional therapeutic agent is administered at a separate dosage at a similar or different frequency relative to an IL-33 antagonist and/or an IL-4R antagonist. In some embodiments, the additional therapeutic agent is administered before, after or concurrently with the IL-33 antagonist and/or the IL-4R antagonist.

In certain embodiments, an IL-33 antagonist and/or an IL-4R antagonist, are administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, an IL-33 antagonist and/or an IL-4R antagonist, are administered every four weeks for 12 weeks, 16 weeks, 20 weeks, 24 weeks, 28 weeks, 32 weeks, 36 weeks, 40 weeks, 44 weeks, 48 weeks or more. In specific embodiments, an IL-33 antagonist and/or an IL-4R antagonist, are administered for at least 24 weeks.

The invention includes methods for treating a subject having mild allergic asthma comprising administering to the subject a loading dose of an antibody or an antigen-binding fragment thereof that specifically binds to IL-4R, and/or an antibody or an antigen-binding fragment thereof that specifically binds to IL-33. In certain embodiments, the methods or uses comprise administering to the subject a plurality of maintenance doses of the antibody(ies) or the antigen-binding fragment(s) thereof, wherein the plurality of maintenance doses are administered during a treatment phase.

### Treatment Populations

For example, “a subject in need thereof” may include, e.g., subjects who, prior to treatment, exhibit (or have exhibited) one or more asthma-associated parameter, such as, e.g., impaired FEV₁ (e.g., less than 2.0 L), impaired FEF25-75%; impaired AM PEF (e.g., less than 400 L/min), impaired PM PEF (e.g., less than 400 L/min), an ACQ5 score of at least 2.5, at least 1 nighttime awakenings per night, and/or a SNOT-22 score of at least 20.

The methods featured in the invention include administering to a subject in need thereof a therapeutic composition comprising an IL-33 antagonist, an IL-4R antagonist, or both an IL-33 antagonist and an IL-4R antagonist. The expression “a subject in need thereof” means a human or non-human animal that exhibits one or more symptoms or indicia of asthma (e.g., allergic asthma), or who has been diagnosed with asthma. In various embodiments, the methods may be used to treat mild, moderate-to-severe, and severe allergic asthma in patients in need thereof, including mild persistent allergic asthma.

In a related embodiment, a “subject in need thereof” may be a subject who, prior to receiving an IL-4R antagonist or both an IL-33 antagonist and an IL-4R antagonist, has been prescribed or is currently taking a SABA or a combination of ICS/LABA. Examples of SABA include, but are not limited to, bitolterol, fenoterol, isoprenaline, isoprotenerol, levosalbutamol, levalbuterol, orciprenaline, metaproterenol, pirbuterol, procaterol, ritodrine, salbutamol, albuterol or terbutaline

Examples of ICS include, but are not limited to, mometasone furoate, budesonide, and fluticasone propionate. Examples of LABA include, but are not limited to, formoterol and salmeterol. Examples of ICS/LABA therapies include, but are not limited to, fluticasone/salmeterol combination therapy and budesonide/formoterol combination therapy. For example, the invention includes methods that comprise administering an IL-4R antagonist or both an IL-33 antagonist and an IL-4R antagonist to a patient who has been taking a course of SABA for two or more weeks immediately preceding the administration of the IL-4R antagonist and/or the IL-33 antagonist on a per needed basis (such prior treatments are referred to herein as “background treatments”). The invention includes therapeutic methods in which background treatments are continued in combination with administration of the IL-4R antagonist and/or the IL-33 antagonist. In yet other embodiments, the amount of the LABA is gradually decreased prior to or after the start of IL-4R antagonist and/or IL-33 antagonist administration. In some embodiments, the methods to treat patients with mild persistent asthma for at least ≥ 12 months are provided. In one embodiment, a patient with mild persistent allergic asthma may be administered an IL-4R antagonist and/or an IL-33 antagonist according to the present methods.

In some embodiments, a “subject in need thereof’ may be a subject with elevated levels of an asthma-associated biomarker. Examples of asthma-associated biomarkers include, but are not limited to, IgE, thymus and activation regulated chemokine (TARC), eotaxin-3, CEA, YKL-40, and periostin. In some embodiments, a “subject in need thereof’ may be a subject with blood eosinophils ≥ 300 cells/µL, 150-299 cells/µL, or < 150 cells/µL. In one embodiment, a “subject in need thereof’ may be a subject with elevated level of bronchial or airway inflammation as measured by the fraction of exhaled nitric oxide (FeNO).

In some embodiments, a “subject in need thereof” is selected from the group consisting of: a subject age 18 years old or older, a subject 12 years or older, a subject age 12 to 17 years old (12 to <18 years old), a subject age 6 to 11 years old (6 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months.

A normal IgE level in healthy subjects is less than about 100 kU/L (e.g., as measured using the IMMUNOCAP® assay [Phadia, Inc. Portage, MI]). Thus, the invention includes methods comprising selecting a subject who exhibits an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L, and administering to the subject a pharmaceutical composition comprising a therapeutically effective amount of an IL-33 antagonist and/or an IL-4R antagonist.

TARC levels in healthy subjects are in the range of 106 ng/L to 431 ng/L, with a mean of about 239 ng/L. (An exemplary assay system for measuring TARC level is the TARC quantitative ELISA kit offered as Cat. No. DDN00 by R&D Systems, Minneapolis, MN.) Thus, the invention involves methods comprising selecting a subject who exhibits an elevated TARC level, which is a serum TARC level greater than about 431 ng/L, greater than about 500 ng/L, greater than about 1000 ng/L, greater than about 1500 ng/L, greater than about 2000 ng/L, greater than about 2500 ng/L, greater than about 3000 ng/L, greater than about 3500 ng/L, greater than about 4000 ng/L, greater than about 4500 ng/L, or greater than about 5000 ng/L, and administering to the subject a pharmaceutical composition comprising a therapeutically effective amount of an IL-33 antagonist and/or an IL-4R antagonist.

Eotaxin-3 belongs to a group of chemokines released by airway epithelial cells, which is upregulated by the Th2 cytokines IL-4 and IL-13 (Lilly et al 1999, J. Allergy Clin. Immunol. 104: 786-790). The invention includes methods comprising administering an IL-33 antagonist and/or an IL-4R antagonist to treat patients with elevated levels of eotaxin-3, such as more than about 100 pg/ml, more than about 150 pg/ml, more than about 200 pg/ml, more than about 300 pg/ml, or more than about 350 pg/ml. Serum eotaxin-3 levels may be measured, for example, by ELISA.

Fractional exhaled NO (FeNO) is a biomarker of bronchial or airway inflammation. FeNO is produced by airway epithelial cells in response to inflammatory cytokines including IL-4 and IL-13 (Alwing et al 1993, Eur. Respir. J. 6: 1368-1370). FeNO levels in healthy adults range from 2 to 30 parts per billion (ppb). An exemplary assay for measuring FeNO is by using a NIOX instrument by Aerocrine AB, Solna, Sweden. The assessment may be conducted prior to spirometry and following a fast of at least an hour. Included here are methods comprising administering an IL-33 antagonist and/or an IL-4R antagonist, to patients with elevated levels of exhaled NO (FeNO), such as more than about 30 ppb, more than about 31 ppb, more than about 32 ppb, more than about 33 ppb, more than about 34 ppb, or more than about 35 ppb.

Carcinoembryogenic antigen (CEA) (also known as CEA cell adhesion molecule 5 [CEACAM5]) is a tumor marker that is found correlated to non-neoplastic diseases of the lung (Marechal et al 1988, Anticancer Res. 8: 677-680). CEA levels in serum may be measured by ELISA. The invention includes methods comprising administering an IL-33 antagonist and/or an IL-4R antagonist, to patients with elevated levels of CEA, such as more than about 1.0 ng/ml, more than about 1.5 ng/ml, more than about 2.0 ng/ml, more than about 2.5 ng/ml, more than about 3.0 ng/ml, more than about 4.0 ng/ml, or more than about 5.0 ng/ml.

YKL-40 (named for its N-terminal amino acids tyrosine (Y), lysine (K)and leucine (L) and its molecular mass of 40 kD) is a chitinase-like protein found to be up regulated and correlated to asthma exacerbation, IgE, and eosinophils (Tang et al 2010 Eur. Respir. J. 35: 757-760). Serum YKL-40 levels are measured by, for example, ELISA. The invention includes methods comprising administering an IL-33 antagonist and/or an IL-4R antagonist, to patients with elevated levels of YKL-40, such as more than about 40 ng/ml, more than about 50 ng/ml, more than about 100 ng/ml, more than about 150 ng/ml, more than about 200 ng/ml, or more than about 250 ng/ml.

Periostin is a secreted matricellular protein associated with fibrosis, and its expression is upregulated by recombinant IL-4 and IL-13 in cultured bronchial epithelial cells and bronchial fibroblasts (Jia et al. (2012) J. Allergy Clin. Immunol.130:647). In human asthmatic patients periostin expression levels correlate with reticular basement membrane thickness, an indicator of subepithelial fibrosis. Id. Included here are methods comprising administering an IL-33 antagonist and/or an IL-4R antagonist, to patients with elevated levels of periostin (e.g., ≥74.4 ng/mL).

Induced sputum eosinophils and neutrophils are well-established direct markers of airway inflammation (Djukanovic et al 2002, Eur. Respire. J. 37: 1S-2S). Sputum is induced with inhalation of hypertonic saline solution and processed for cell counts according to methods known in the art, for example, the guidelines of European Respiratory Society.

In some embodiments, the subjects are stratified into the following groups: a blood eosinophil count (high blood eosinophils) ≥300 cells/µL (HEos) or 300 - 499 cells/µL or ≥500 cells/µL, a blood eosinophil count of 200 to 299 cells/µL (moderate blood eosinophils), or a blood eosinophil count <200 cells/µL (low blood eosinophils), and are administered an IL-33 antagonist and/or an IL-4R antagonist, at a dose or dosing regimen based upon the eosinophil level.

In some embodiments, a subject has “eosinophilic phenotype” asthma defined by a blood eosinophil count of ≥150 cells/µL, a blood eosinophil count of ≥300 cells/µL, or a blood eosinophil count of ≥500 cells/µL, and are administered an IL-33 antagonist and/or an IL-4R antagonist.

In some embodiments, a subject has “periostin phenotype” asthma defined by a high blood periostin level as defined herein, and are administered an IL-33 antagonist and/or an IL-4R antagonist.

In some embodiments, a “subject in need thereof” is a subject that is a clinically stable, non-smoker with mild persistent allergic asthma who requires only inhaled short-acting β2 agonist (SABA) use on a per needed basis to control asthma symptoms and who is allergic to house dust mite (HDM) allergen, as determined by a skin prick test.

### Methods for Assessing Pharmacodynamic Asthma-Associated Parameters

The invention also includes methods for assessing one or more pharmacodynamic asthma-associated parameters in a subject in need thereof, caused by administration of a pharmaceutical composition comprising an IL-33 antagonist, an IL-4R antagonist, or an IL-33 antagonist and an IL-4R antagonist. A reduction in the incidence of an allergic asthma exacerbation (as described above) or an improvement in one or more asthma-associated parameters (as described above) may correlate with an improvement in one or more pharmacodynamic asthma-associated parameters; however, such a correlation is not necessarily observed in all cases.

Examples of “pharmacodynamic asthma-associated parameters” include, for example, the following: (a) biomarker expression levels; (b) serum protein and RNA analysis; (c) induced sputum eosinophils and neutrophil levels; (d) exhaled nitric oxide (FeNO); and (e) blood eosinophil count. An “improvement in a pharmacodynamic asthma-associated parameter” means, for example, a decrease from baseline of one or more biomarkers, such as periostin, TARC, eotaxin-3 or IgE, a decrease in sputum eosinophils or neutrophils, FeNO, periostin or blood eosinophil count. As used herein, the term “baseline,” with regard to a pharmacodynamic asthma-associated parameter, means the numerical value of the pharmacodynamic asthma-associated parameter for a patient prior to or at the time of administration of a pharmaceutical composition described herein.

To assess a pharmacodynamic asthma-associated parameter, the parameter is quantified at baseline and at a time point after administration of the pharmaceutical composition. For example, a pharmacodynamic asthma-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic asthma-associated parameter (e.g., an increase or decrease, as the case may be, depending on the specific parameter being measured).

In certain embodiments, administration of an IL-33 antagonist, an IL-4R antagonist or an IL-33 antagonist and an IL-4R antagonist, to a patient causes a change, such as a decrease or increase, in expression of a particular biomarker. Asthma-associated biomarkers include, but are not limited to, the following: (a) total IgE; (b) thymus and activation-regulated chemokine (TARC); (c) YKL-40; (d) carcinoembryonic antigen in serum; (e) eotaxin-3 in plasma; and (f) periostin in serum. For example, administration of an IL-33 antagonist and/or an IL-4R antagonist, to an asthma patient can cause one or more of a decrease in TARC or eotaxin-3 levels, or a decrease in total serum IgE levels. The decrease can be detected at week 1, week 2, week 3, week 4, week 5, or longer following administration of the IL-33 antagonist, the IL-4R antagonist or the IL-33 antagonist and the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-33 antagonist and/or an IL-4R antagonist, IL-4/IL-13 signaling, asthma, atopy or eosinophilic diseases (e.g., by measuring soluble IL-4Rα, IL-4, IL-13, periostin and the like). In some embodiments, RNA samples are used to determine RNA levels (non-genetic analysis), e.g., RNA levels of biomarkers, and in other embodiments, RNA samples are used for transcriptome sequencing (e.g., genetic analysis).

### EXAMPLE

The following example is put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-33 antagonist used in the following Example is the human anti-IL-33 antibody named SAR440340, which is also referred to as REGN3500 or by its international nonproprietary name (INN), itepekimab. The exemplary IL-4R antagonist used in the following Example is the human anti-IL-4R antibody named dupilumab (i.e., DUPIXENT®).

### Example 1. A Randomized, Placebo-controlled, Parallel Panel Study to Assess the Effects of REGN3500, Dupilumab, and Combination of REGN3500 Plus Dupilumab on Markers of Inflammation After Bronchial Allergen Challenge in Patients With Allergic Asthma

### Overview and Study Rationale

REGN3500 and dupilumab are fully human monoclonal antibodies (mAbs). Dupilumab is an anti-interleukin-4 receptor alpha subunit (IL-4Rα) mAb. REGN3500 targets IL-33, a pro-inflammatory cytokine that initiates and amplifies innate and adaptive inflammatory cascades (Cayrol et al. IL-33: an alarmin cytokine with crucial roles in innate immunity, inflammation and allergy. Curr Opin Immunol. 2014. 31:31-37.)

This study is to evaluate the treatment effects of REGN3500, dupilumab, and REGN3500 plus dupilumab combination, as compared with placebo, and the effect of inhaled corticosteroids on the allergic inflammatory pathways triggered by an inhaled house dust mite (HDM) bronchial allergen challenge (BAC), in HDM-sensitive adult patients with mild asthma. The inhaled BAC model has been effectively used in asthma drug development for over 30 years (Diamant et al. Inhaled allergen bronchoprovocation tests. J Allergy Clin Immunol. 2013. 132:1045-1055 e1046, Fahy et al. Analysis of cellular and biochemical constituents of induced sputum after allergen challenge: a method for studying allergic airway inflammation. J Allergy Clin Immunol. 1994. 93:1031-1039, Inman et al. Dose-dependent effects of inhaled mometasone furoate on airway function and inflammation after allergen inhalation challenge. Am J Respir Crit Care Med. 2001.164:569-574.)

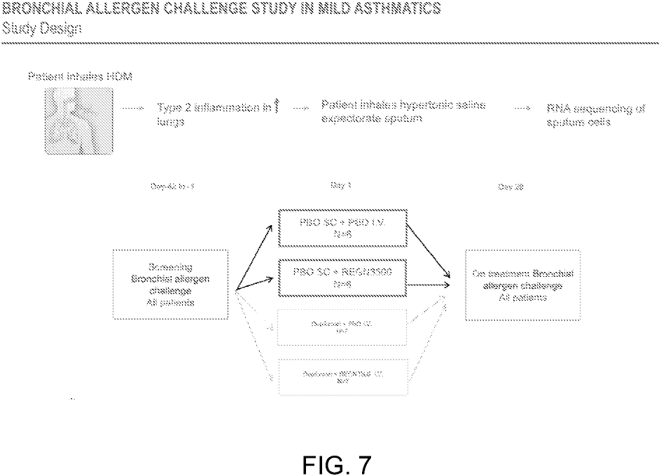

Bronchial allergen challenge involves patient inhalation of allergen that results in a biphasic airway response, which is characterized by an early (30 minutes to 2 hours post-allergen challenge) and a late (approximately 3 to 8 hours post-allergen challenge) decline in forced expiratory volume over 1 second (FEV1). This model facilitates the evaluation of allergic inflammatory response via measurement of changes in cell content, cytokine production, and mRNA inflammatory signatures in bronchoalveolar lavage, bronchial biopsies, or induced sputum (Zuiker et al. Sputum RNA signature in allergic asthmatics following allergen bronchoprovocation test. Eur Clin Respir J. 2016. 3:31324.) As shown in FIG. 7, this study leverages the BAC model to evaluate treatment-induced changes in allergic inflammation as measured in induced sputum, with a particular focus on changes in targeted selected mRNA signatures.

A key feature of this study design is the requirement of a pre-treatment BAC to allow intra-patient comparison of pre- and post-treatment effects on BAC.

The purpose of this study is to evaluate the effects of REGN3500, dupilumab, and REGN3500 plus dupilumab combination on the molecular mechanisms involved in allergic inflammation in the asthmatic airway, which are thought to contribute to asthma pathogenesis. To accomplish this goal, this study explores the expression of select markers of inflammation in the sputum of patients with mild allergic asthma, induced with a controlled BAC using HDM. Comparison of the effects of treatment with REGN3500, dupilumab, or REGN3500 plus dupilumab combination on changes in the expression of inflammatory pathway molecules in the sputum provides data on whether REGN3500 plus dupilumab combination has an additive effect.

### Objectives

The primary objective is to assess the effects of REGN3500, dupilumab, and the combination of REGN3500 plus dupilumab, compared with placebo, on changes in inflammatory gene expression signatures in sputum induced after a bronchial allergen challenge (BAC) in adults with mild allergic asthma, at week 4 after treatment initiation compared with those at screening.

The secondary objectives of the study are: to assess the safety and tolerability of limited doses of REGN3500 and the combination of REGN3500 plus dupilumab in adult patients with mild allergic asthma who undergo BACs; to assess the pharmacokinetic (PK) profile of REGN3500 in adult patients with mild allergic asthma who undergo BACs; to assess the immunogenicity of REGN3500 and dupilumab in adult patients with mild allergic asthma who undergo serial BACs; to assess target engagement of REGN3500 via measurement of total interleukin-33 (IL-33) levels in serum in adult patients with mild allergic asthma who undergo serial BACs; and to assess the effect of fluticasone on changes in inflammatory gene expression signatures in sputum induced after a BAC in adults with mild allergic asthma at day 4 after treatment initiation compared with that at screening.

The exploratory objectives of the study were in adult patients with mild allergic asthma: to assess the effects of REGN3500, dupilumab, combination of REGN3500 plus dupilumab, and placebo on changes in an inflammatory gene expression signatures in sputum induced after a BAC at week 8 after treatment initiation compared with those at screening; to assess the effects of REGN3500, dupilumab, combination of REGN3500 plus dupilumab, and placebo on changes in inflammatory cytokine proteins in sputum induced after a BAC at week 4 and week 8 after treatment initiation compared with those at screening; to assess the potential effects of REGN3500, dupilumab, combination of REGN3500 plus dupilumab, and placebo on changes in serum markers of IL-33 pathway activation after a BAC, at week 4 and week 8 after treatment initiation compared with those at screening; to assess the effects of REGN3500, dupilumab, combination of REGN3500 plus dupilumab, and placebo on lung function parameters, including changes in pre-BAC FEV1 and fractional exhaled nitric oxide (FeNO) and change in FEV1 area under the curve (AUC) at 0 to 2 hours and 3 to 8 hours after BAC, at week 4 and week 8 after treatment initiation compared with those at screening; and to compare the effect of REGN3500, dupilumab, or combination of REGN3500 plus dupilumab with that of inhaled fluticasone propionate on changes in sputum gene expression signatures, sputum cytokines, serum biomarkers of IL-33 and IL-4R activity, and on changes in FeNO and in FEV1 AUC after BAC, at week 4 and week 8 after treatment initiation compared with those at screening.

### Study Design

In this study, the inhaled BAC model is used to assess the impact of REGN3500, dupilumab, and combination of REGN3500 plus dupilumab on airway inflammation. The inhaled BAC model is a well-established and reproducible model of induced allergic airway inflammation, which allows assessment of drug treatment effects at a functional, cellular, and molecular level. Previous studies have demonstrated that application of this model may be used to evaluate treatment response via measurement of mRNAs of inflammatory genes in induced sputum (Zuiker et al. Sputum RNA signature in allergic asthmatics following allergen bronchoprovocation test. Eur Clin Respir J. 2016. 3:31324.) Several published reports have demonstrated that the inflammatory response following a BAC results in an upregulation of type 2 cytokines in both bronchial alveolar fluid and sputum (Erin et al. Optimized dialysis and protease inhibition of sputum dithiothreitol supernatants. Am J Respir Crit Care Med. 2008. 177:132-141, Huang et al. IL-13 expression at the sites of allergen challenge in patients with asthma. J Immunol. 1995. 155:2688-2694.) The hypothesis of this study is that the acute upregulation of type 2 inflammation from the BAC model allows evaluation of the effects of REGN3500, dupilumab, and the combination of REGN3500 plus dupilumab on this allergic airway inflammation.

This is a 2-part study, consisting of a randomized, double-blind, placebo-controlled component (Part 1) and an open-label component (Part 2).

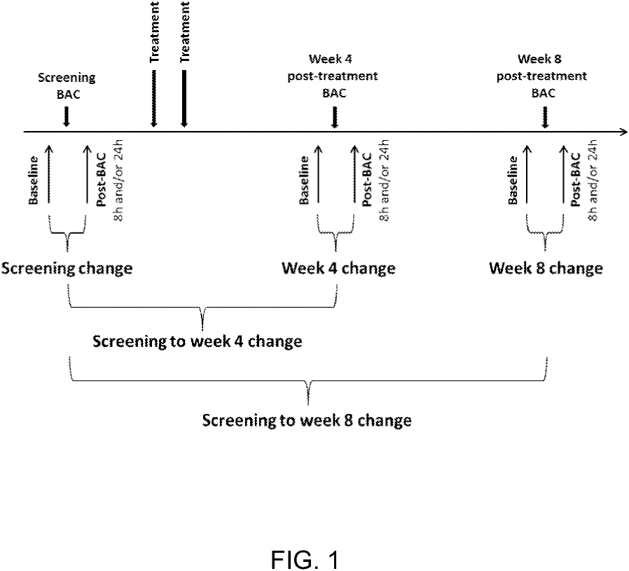

As shown in FIG. 1, in Part 1 of the study, patients are randomized to receive REGN3500 (intravenous (IV), single dose), dupilumab (subcutaneous (SC), 2 doses, 14 days apart), the combination of REGN3500 (IV, single dose) plus dupilumab (SC, 2 doses, 14 days apart), or placebo. Enrolled patients undergo BAC during the screening period, and at 4 and 8 weeks after administration of the first doses of study drug(s). The effects of the study drugs on BAC-induced lung inflammation are assessed via measurements of sputum molecular signatures (mRNAs and proteins). The sputum molecular signatures are assessed before BAC (baseline) and after BAC (post-BAC) at screening and at weeks 4 and 8 after treatment initiation (FIG. 1). The BAC-induced difference in the sputum signatures before and after BAC are evaluated at screening (screening change) and at weeks 4 and 8 post-treatment (week 4 change and week 8 change) will be observed. Since both REGN3500 and dupilumab display long half-lives, BAC change is evaluated at both week 4 and week 8 in the current study, to monitor the durability of the potential impact of study drug treatment on airway inflammation. The effect of study drug treatment on induced sputum gene signatures is evaluated by the difference between the BAC-induced screening change and the BAC-induced week 4 change (screening to week 4 change) and by the difference between the BAC-induced screening change and the BAC-induced week 8 change (screening to week 8 change).

The BAC model has been used in the development of effective, potent anti-inflammatory agents for asthma such as inhaled corticosteroids (Hansel et al. The allergen challenge. Clin Exp Allergy. 2002. 32:162-167, Inman et al. Dose-dependent effects of inhaled mometasone furoate on airway function and inflammation after allergen inhalation challenge. Am J Respir Crit Care Med. 2001. 164:569-574, and Ravensberg et al. Validated safety predictions of airway responses to house dust mite in asthma. Clin Exp Allergy. 2007. 37:100-107.) Although the typical endpoint of these studies is the change in the late phase FEV1 (drop in FEV1 seen typically 4 to 8 hours post allergen exposure), the model also has demonstrated good test retest reproducibility of inflammatory markers measured in the airway, post-BAC (Fahy et al. Analysis of cellular and biochemical constituents of induced sputum after allergen challenge: a method for studying allergic airway inflammation. J Allergy Clin Immunol. 1994. 93:1031-1039, Inman et al. Dose-dependent effects of inhaled mometasone furoate on airway function and inflammation after allergen inhalation challenge. Am J Respir Crit Care Med. 2001. 164:569-574, and Zuiker et al. Kinetics of TH2 biomarkers in sputum of asthmatics following inhaled allergen. Eur Clin Respir J. 2015. 2.)

To improve the ability to interpret results with small numbers of patients, each patient undergoes a BAC during the screening period. In order to be included in the study, each patient must demonstrate both an early FEV1 decline (from 0 to 30 minutes after BAC) as well as a late FEV1 decline (3 to 8 hours after BAC). Previous studies have demonstrated that patients who have both early and late FEV₁ decline have higher levels of type 2 cytokines at the late phase than do patients without a FEV1 decline in this period. The second BAC in Part 1 of the study is performed at approximately 4 weeks after the first dose of study treatment. A third BAC is proposed at approximately 8 weeks after treatment initiation to provide an assessment of durability of effect. These data provides the ability to create a PK/pharmacodynamic (PD) model of airway effects of REGN3500, dupilumab, and the combination of REGN3500 plus dupilumab.

Data from 6 patients is expected to provide sufficient statistical power to detect a treatment effect. Due to potential technical difficulties in successful completion of all procedures, up to 8 patients will be enrolled for each treatment group. Data from 6 patients per group is expected to allow >80% power to detect a treatment effect size of 2.5- to 5- fold on designated mRNA cytokine expression.

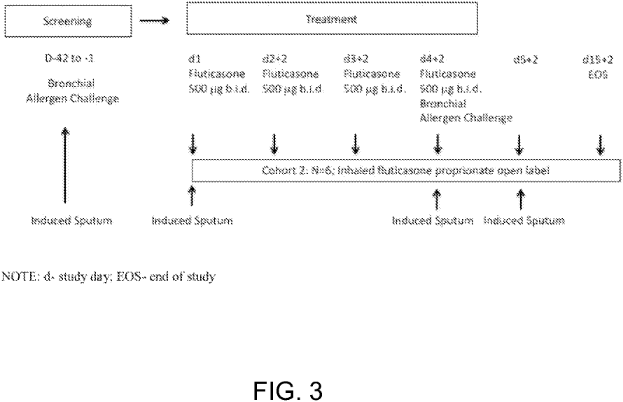

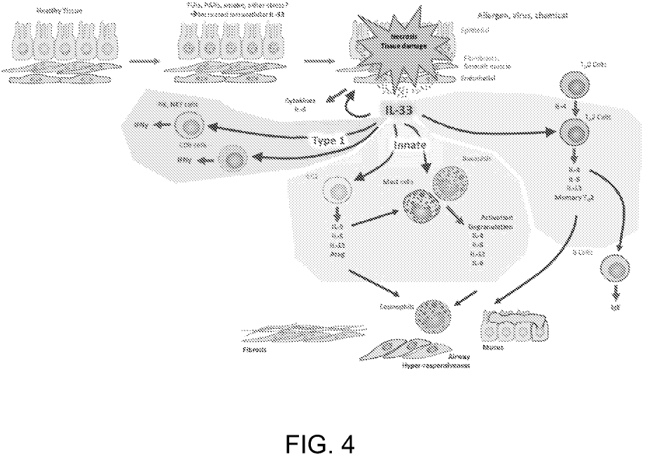

As shown in FIG. 3, in Part 2 of this study, patients undergo a screening BAC, followed by a short course of open-label, high dose inhaled fluticasone propionate. A second BAC is given 4 days after initiation of inhaled fluticasone propionate treatment. This part of the study serves as a positive control to confirm previously reported corticosteroid effects on mRNA allergic inflammatory signature (Zuiker et al. Sputum RNA signature in allergic asthmatics following allergen bronchoprovocation test. Eur Clin Respir J. 2016. 3:31324) and to provide a comparator to evaluate the effects of the study drugs on allergen induced inflammation.

This is a phase 1b study with 2 distinct parts. Part 1 is of 42 weeks duration, excluding the screening period. Part 2 is of 2 weeks duration, excluding the screening period. Part 1 and Part 2 are run concurrently.

Part 1 of this study is a randomized, double-blind, placebo-controlled, double-dummy parallel group study in mild persistent allergic asthmatic patients to assess the effect of REGN3500, dupilumab, REGN3500 plus dupilumab combination, or placebo on lung inflammation (as measured by sputum cytokine mRNAs) and measurements of the late phase inflammatory airway response to a BAC using HDM. Patients are randomized to one of the following treatment groups: REGN3500 (IV single dose); dupilumab (SC 2 doses, Q2W); combination REGN3500 (IV single dose) plus dupilumab (SC 2 doses, Q2W), and placebo (IV single dose and SC 2 doses, Q2W).

The effect of treatment on lung inflammation and on measurements of the late phase inflammatory airway response to a BAC with HDM is evaluated. Patients are followed up from days 58 to the end of study visit on day 293.

Part 2 involves open-label treatment with a short course of inhaled fluticasone propionate. Patients are administered 8 doses of inhaled fluticasone propionate over 4 days and are followed up through the end of study visit on day 15. The effects of fluticasone propionate on sputum cytokine mRNAs and measurements of the late phase airway response are used as a positive control for comparison with those measurements in Part 1.

Anti-drug antibody variables include status (positive or negative) and titer as follows: total number of patients negative in ADA assay at all time points analyzed, total number of patients positive in the ADA assay at all time points analyzed, total number of patients with pre-existing immunoreactivity, total number of patients with treatment-emergent ADA response, total number of patients with treatment-boosted ADA response, and titer category: low (titer<1,000), moderate (1,000 ≤ titer ≤10,000), and high (titer >10,000).

### Screening

All patients included in this study are clinically stable, non-smokers with mild persistent allergic asthma who require only short-acting inhaled β2 agonist use on a per need basis to control asthma symptoms and who are atopic to HDM, as determined by a skin prick test.

During the screening period (day -28 to day -1), pre-study procedures are performed on potential study patients. Patients who meet all other entry criteria undergo an inhaled HDM allergen challenge to provide baseline assessment of airway allergic response and to determine eligibility. During the screening period (day -28 to day -1), patients are required to show both an early and a late phase allergic reaction during the screening BAC.

Patients are required to tolerate sputum induction and to provide an adequate sputum sample during screening. The definition for adequate sputum sample will be given in the study manual. Patients who fail to produce an adequate sputum sample pre-BAC during the screening process may be rescreened for sputum induction with the investigator’s approval. Patients who fail to produce sputum post-BAC will be ineligible to enroll in the study.

The dose of allergen required to demonstrate an appropriate EAR for an individual patient during the screening challenge is used to calculate the dose regimen of allergen for this particular patient in the post-treatment BACs (day 29 and day 57 after treatment initiation for Part 1 and day 4 after treatment initiation for Part 2). To be included in the study, patients must demonstrate an adequate LAR defined as a drop in FEV1 of 15% or more from pre-BAC FEV1 (however, patient must not drop his/her FEV₁ below 25% predicted or <1.4 L). Procedures during the screening BAC are similar to the procedures performed in the treatment phase of the trial.

### Study Part 1

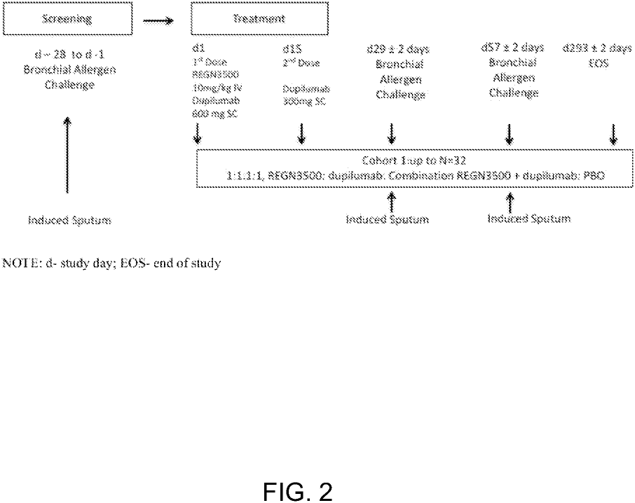

As shown in FIG. 2, eligible patients (up to 32 in total) are randomized 1:1:1:1 to receive REGN3500, dupilumab, REGN3500 plus dupilumab combination, or placebo. Because the treatment groups in this study include a first clinical dosing of REGN3500 plus dupilumab combination, Part 1 is performed in 2 phases to address any potential safety issues. The first 8 patients are randomized 1:1:1:1 to receive study drug. On day 1: patients in the REGN3500 group receive REGN3500 IV 10 mg/kg, followed by dupilumab-matching placebo SC, patients in the dupilumab group receive REGN3500-matching placebo IV followed by a loading dose of dupilumab SC 600 mg (2 injections of 300 mg), patients in the REGN3500 plus dupilumab combination will receive REGN3500 10 mg/kg IV, followed by dupilumab SC 600 mg (2 injections of 300 mg), and patients in the placebo group will receive REGN3500-matching placebo IV, followed by 2 injections of dupilumab-matching placebo SC.

On day 15 (week 2), patients assigned to the dupilumab group or the REGN3500 plus dupilumab combination group receive dupilumab SC 300 mg and all other patients receive dupilumab-matching placebo SC.

These first 8 patients complete safety assessments through day 24 (visit 7) of the study. The Regeneron Safety Oversight Committee (RSOC) will perform an unblinded safety review of the data. The remaining approximately 24 patients are enrolled once the RSOC has reviewed the safety data and approves further enrollment. These remaining patients are randomized and receive treatment as described for the first 8 patients.

All enrolled patients return to the clinic on day 24± 2 days and on day 52 ± 2 days to provide a baseline induced sputum sample. If an adequate sputum sample cannot be obtained, patients may return 72 hours later for a second attempt to produce baseline sputum. Pre-BAC sputum collection must occur at least 72 hours prior to the BAC. Seventy-two hours after the baseline induced sputum sample is collected, on day 29 ± 2 days and on day 57 ± 2 days, respectively, patients return to the clinic, and measurements of FEV1, and FeNO are performed prior to undergoing BAC with HDM. Sputum induction is performed 8 hours and 24 hours after inhaled BAC at both the days 29 ± 2 (week 4) and 57 ± 2 days (week 8) visits. Patients are monitored at least 8 hours post-BAC in house and are permitted to leave the study site when deemed stable by the study physician. The washout period between BACs will be at least 21 days.

Measurements of pulmonary function as assessed by spirometry, sputum mRNA, sputum cytokines, FeNO, and additional serum markers of IL-33 and IL-4R activity will be performed, as detailed in the Schedule of Events shown in Table 1 below. Patients will be followed after BAC at scheduled visits until the end of study visit (day 293).

TABLE 1

Schedule of Events for Part 1

| | Screening | | | | Treatment | | Post-treatment | | | | | | | | | | |
|---|---|---|---|---|---|---|---|---|---|---|---|---|---|---|---|---|---|
| | | Induced Sputum & BAC | | | | | Induced Sputum & BAC | | | | Induced Sputum & BAC | | | | | | EOS/ ET* |
| Study Procedure | Visit 1 | Visit 2 | Visit 3 | Visit 4 | Visit 5 | Visit 6 | Visit 7 | Visit 8 | Visit 9 | Visit 10 | Visit 11 | Visit 12 | Visit 13 | Visit 14 | Visit 15 | Visit 16 | Visit 17 |
| Study Day (±window days) | -28 to -1 | | | | 1 | 15 (±2) | 24 (±2) | 29 (±2) | 30 (±2) | 43 (±2) | 52 (±2) | 57 (±2) | 58 (±2) | 86 (±2) | 113 (±2) | 203 (±2) | 293 (±2) |
| Administrative | | | | | | | | | | | | | | | | | |
| Informed Consent | X^{a} | | | | | | | | | | | | | | | | |
| Genomics Sub-study Consent^{a} | X^{b} | | | | | | | | | | | | | | | | |
| Medical History | X | | | | | | | | | | | | | | | | |
| Demographics | X | | | | | | | | | | | | | | | | |
| Inclusive Exclusion | X | | | | X | | | | | | | | | | | | |
| Study Drug | | | | | | | | | | | | | | | | | |
| Randomization | | | | | X | | | | | | | | | | | | |
| Study Drug Administration^{e} | | | | | X^{e2,f2,} ^{g1,m} | X^{e2,f2,} ^{g1} | | | | | | | | | | | |
| Laboratory Testing (all baseline samples collected pre-dose) | | | | | | | | | | | | | | | | | |
| Hepatitis (HB_{S}AG, HBcAb, HCV), and HIV Screen, TB blood test^{d,e1,f1} | X | | | | | | | | | | | | | | | | |
| Urine Drug Screen and alcohol breath tests (as per site SOP) | X | | | | X | | | | | | | | | | | | |

| | Screening | | | | Treatment | | Post-treatment | | | | | | | | | | |
|---|---|---|---|---|---|---|---|---|---|---|---|---|---|---|---|---|---|
| | | Induced Sputum & BAC | | | | | Induced Sputum & BAC | | | | Induced Sputum & BAC | | | | | | EOS/ ET* |
| Study Procedure | Visit 1 | Visit 2 | Visit 3 | Visit 4 | Visit 5 | Visit 6 | Visit 7 | Visit 8 | Visit 9 | Visit 10 | Visit 11 | Visit 12 | Visit 13 | Visit 14 | Visit 15 | Visit 16 | Visit 17 |
| Study Day (±window days) | -28 to -1 | | | | 1 | 15 (±2) | 24 (±2) | 29 (±2) | 30 (±2) | 43 (±2) | 52 (±2) | 57 (±2) | 58 (±2) | 86 (±2) | 113 (±2) | 203 (±2) | 293 (±2) |
| Administrative | | | | | | | | | | | | | | | | | |
| Hematology Panel^{d,e1,f1} | X | | X | | X^{e2,f2} | X^{e2,f2} | | X | | | | X | | X | X | X | X |
| Laboratory Chemistry (including ionized calcium)^{d,e1,f1} | X | | X | | X^{e2,f2} | X^{e2,f2} | | X | | | | X | | X | X | X | X |
| Intact Parathyroid Hormone (iPTH)^{d,e1,f1} | X | | | | X^{e2,f2} | | | X | | | | X | | | X | X | X |
| 25-Hydroxy Vitamin D^{d,e1,f1} | X | | | | X^{e2,f2} | | | X | | | | X | | | X | X | X |
| Serum Pregnancy Test^{d,e1} | X | | | | | | | | | | | | | | X | X | X |
| Urine Pregnancy Test | X | | X | | X | X | | X | | | | X | | | X | X | X |
| FSH (amneorrheic women only)^{d,e1} | X | | | | | | | | | | | | | | | | |
| Urinalysis | X | | | | X | | | X | | | | X | | | X | X | X |
| Clinical Procedural Assessments | | | | | | | | | | | | | | | | | |
| Vital Signs^{e} | X^{e1} | X | X^{e1} | X | X^{e2} | X^{e2} | X^{e1} | X^{e1} | X | X | X | X^{e1} | X | X^{e1} | X^{e1} | X^{e1} | X^{e1} |
| Physical Examination | X | | | | | | | | | | | | | | X | X | X |
| Weight | X | | | | X | | | X | | | | X | | X | X | X | X |
| Height | X | | | | | | | | | | | | | | | | |

| | Screening | | | | Treatment | | Post-treatment | | | | | | | | | | |
|---|---|---|---|---|---|---|---|---|---|---|---|---|---|---|---|---|---|
| | | Induced Sputum & BAC | | | | | Induced Sputum & BAC | | | | Induced Sputum & BAC | | | | | | EOS/ ET* |
| Study Procedure | Visit 1 | Visit 2 | Visit 3 | Visit 4 | Visit 5 | Visit 6 | Visit 7 | Visit 8 | Visit 9 | Visit 10 | Visit 11 | Visit 12 | Visit 13 | Visit 14 | Visit 15 | Visit 16 | Visit 17 |
| Study Day (±window days) | -28 to -1 | | | | 1 | 15 (±2) | 24 (±2) | 29 (±2) | 30 (±2) | 43 (±2) | 52 (±2) | 57 (±2) | 58 (±2) | 86 (±2) | 113 (±2) | 203 (±2) | 293 (±2) |
| Administrative | | | | | | | | | | | | | | | | | |
| Electrocardiogram^{f,f1} | X | | X | | X^{f2} | X^{f2} | | X | | | | X | | | | | X |
| Adverse EffectsX | X | X | X | X | X | X | X | X | X | X | X | X | X | X | X | X | X |
| Concomitant Medications | X | X | X | X | X | X | X | X | X | X | X | X | X | X | X | X | X |
| Pharmacokinetic (PD) and Anti-Drug Antibody(ADA) | | | | | | | | | | | | | | | | | |
| Serum sample for REGN3500 drug concentration^{d,g} | | | | | X^{g1} | X^{g1} | | X | | X | | X | | X | X | X | X |
| Serum for dupilumab drug concentration^{d,g} | | | | | X^{g1} | X^{g1} | | X | | X | | X | | X | X | | |
| ADA (anti-REGN3500)^{d,g} | | | | | X^{g1} | | | | | | | | | | | | x |
| ADA (anti-dupilumab) sample^{d,g} | | | | | X^{g1} | | | | | | | | | | X | | |
| Procedures | | | | | | | | | | | | | | | | | |
| HDM Skin Prick Test (standard & skin sensitivity test) | X | | | | | | | | | | | | | | | | |
| In-clinic Spirometry^{h} | Xⁱ¹ | X | X^{i1,m} | X^{i1,m} | X^{i1,m} | Xⁱ¹ | X | Xⁱ¹ | Xⁱ¹ | Xⁱ¹ | X | X^{i1,m} | X^{i1,m} | X^{i1,m} | X^{i1,m} | X^{i1,m} | X^{i1,m} |
| FeNO^{i,i1} | Xⁱ¹ | | X^{i2,i3} | X^{i2,i3} | X | X | | X^{i2,i3} | X^{i2,i3} | X | | X^{i2,i3} | X^{i2,i3} | X | X | X | X |

| | Screening | | | | Treatment | | Post-treatment | | | | | | | | | | |
|---|---|---|---|---|---|---|---|---|---|---|---|---|---|---|---|---|---|
| | | Induced Sputum & BAC | | | | | Induced Sputum & BAC | | | | Induced Sputum & BAC | | | | | | EOS/ ET* |
| Study Procedure | Visit 1 | Visit 2 | Visit 3 | Visit 4 | Visit 5 | Visit 6 | Visit 7 | Visit 8 | Visit 9 | Visit 10 | Visit 11 | Visit 12 | Visit 13 | Visit 14 | Visit 15 | Visit 16 | Visit 17 |
| Study Day (±window days) | -28 to -1 | | | | 1 | 15 (±2) | 24 (±2) | 29 (±2) | 30 (±2) | 43 (±2) | 52 (±2) | 57 (±2) | 58 (±2) | 86 (±2) | 113 (±2) | 203 (±2) | 293 (±2) |
| Administrative | | | | | | | | | | | | | | | | | |
| Methacholine challenge | Xⁱ¹ | | | | | | | | | | | | | | | | |
| Induced Sputum^{j} | | X^{j1} | X^{i2,j2,} ^{m} | X^{i2,j2,} ^{m} | | | X^{j1} | X^{i3,j2,} ^{m} | X^{i3,j2,} ^{m} | | X^{j1} | X^{i3,j2,} ^{m} | X^{i3,j2,} ^{m} | | | | |
| BAC with HDM^{k} | | | X^{i3,j2,} ^{m,} ⁿ¹ | | | | | X^{i3,j2,} ^{m,n1} | | | | X^{i3,j2,} ^{m,n1} | | | | | |
| Sample Collection and Biomarkers^{d} | | | | | | | | | | | | | | | | | |
| DNA (optional)^{d,1} | | | | | X | | | | | | | | | | | | |
| Whole blood RNA Sample (optional)^{d,m} | | | X | X | X | | | X | X | | | X | X | | | | |
| Serum and plasma sST2, calcitonin, MMP12, TARC, PARC, eotaxin-3^{d,n} | X | | Xⁿ¹ | Xⁿ² | X | X | | Xⁿ¹ | Xⁿ² | | | Xⁿ¹ | Xⁿ² | X | X | X | X |
| Serum total IL-33^{d,n} | X | | Xⁿ¹ | Xⁿ² | X | X | | Xⁿ¹ | Xⁿ² | X | | Xⁿ¹ | Xⁿ¹ | X | X | X | X |
| Future biomedical research serum and plasma samples^{d} | X | | | | X | | | X | | | | X | | X | X | X | X |

*EOS/ET: End of Stud/ Early Termination; BAC: Bronchial allergen challenge; HDM: House dust mite

### Footnotes for the Schedule of Events Table 1

a. Informed consent may be obtained on a separate visit prior to the screening period.
b. Patients must sign a separate informed consent form (ICF) prior to collection of samples for DNA analysis. Patients who meet the inclusion/exclusion criteria are eligible to enroll in the study regardless of whether they choose to participate in the genomics sub-study.
c. REGN3500 (or matching placebo) is administered as a single IV dose on day 1. Dupilumab (or matching placebo) is administered SC in 2 injections on day 1 and in 1 injection on day 15. Intravenous administration of study drug is performed prior to SC administration of study drug, with at least 1 hour between the IV and SC dosing. On day 1 and day 15, following the administration of SC dose of dupilumab, patients are observed for 8 hours before dismissal. Patients may eat 2 hours after completion of infusion.
d. All blood samples (safety laboratory, PK, ADA, biomarker, and future biomedical research) should be collected after an overnight fast of at least 8 hours.
e. Vital signs measurements:
   e1. Vital signs should be collected prior to any blood draw.
   e2. On study day 1 and day 15, vital signs are collected prior to administration of study drug, immediately (within 10 minutes) after the end of IV infusion and prior to SC injection of study drug, and 1, 2, 4, and 8 hours after the end of injection.
f. Details on ECG measurements:
   f1. Electrocardiogram are performed before blood is drawn during visits that require blood draws.
   f2. On day 1 and day 15, ECG is performed prior to, immediately (within 10 minutes) after the end of the IV infusion and 4 hours after the end of the SC injection of study drug.
g. All blood samples for PK and ADA are collected prior to initiation of study drug administration.
   g1. On day 1 and day 15, samples for safety laboratory, ADA, and biomarkers are collected prior to the start of the study drug administration (fasted).
h. In-clinic spirometry is performed in accordance with American Thoracic Society criteria standards of acceptability quality control with a standard spirometer (Miller et al. Standardisation of spirometry. Eur Respir J. 2005. 26:319-338).
i. FeNO should be conducted following a fast of at least 1 hour.
   i1. FeNO measurement must be performed before spirometry and methacholine challenge.
   i2. FeNO measurement must be performed before sputum induction.
   i3. On challenge days (visit 3, visit 8, and visit 12), FeNO is measured before BAC is performed, and at 8 hours and 23 hours post-BAC.
j. Days in which sputum may be collected are shown in gray.
   j1. Pre-allergen screening sputum samples are collected at least 48 hours after the methacholine challenge performed at visit 1, and 72 hours prior to the BAC performed at visit 3. Other pre-allergen sputum samples are collected at least 72 hours prior to the BAC. If a pre-BAC sample cannot be collected on a first attempt, a repeat attempt may be performed at least 72 hours later.
   j2. On BAC days (visit 3, visit 8, visit 12), sputum is also collected at 8 hours after the challenge, and on the following day, approximately 24 hours after the BAC (visit 4, visit 9 and visit 13).
l. DNA sample should be collected on day 1. However, DNA may be collected at any visit during the course of the trial.
m. Whole blood for extraction of RNA samples should only be collected at the indicated study visits prior to BAC, spirometry, or induced sputum procedures on the indicated day. Whole blood for RNA samples should be collected prior to drug administration on study drug treatment days.
n. Biomarker samples are collected at the specified times during the study, with the following notes:
   n1. Biomarkers and total IL-33 samples on BAC days (visit 3, visit 8, and visit 12) should be collected pre-BAC and at 8 hours post-BAC (after collection of the 8 hour post-BAC sputum sample).
   n2. Biomarkers samples collected on the day following BAC (visit 4, visit 9, and visit 13) should be collected 24 hours post-BAC, after collection of the sputum sample.

### Study Part 2

As shown in FIG. 3, in Part 2 of the study, approximately 6 patients receive fluticasone propionate inhaled 500 µg (2 puffs of 250 µg) per dose, twice per day for 4 days (total 8 doses), starting on day 1. Patients receive drug in the clinic on day 1 and inhaler technique is reviewed. Patients are sent home to self-administer drug for days 2 and 3. Induced sputum samples are collected after the second dose of fluticasone on day 1. On day 4, patients receive fluticasone propionate (in the clinic) prior to BAC procedures and patients self-administer the second dose that day after sputum induction (either in the clinic or at home). Patients are given an inhaled BAC with HDM similar to that in study Part 1 after receiving the seventh of 8 doses of fluticasone propionate. Induced sputum samples are collected 8 hours, and 24 hours after the HDM challenge on day 4. Measurements of pulmonary function as assessed by spirometry, sputum mRNA, sputum cytokines, FeNO, and additional serum markers of IL-33 and IL-4R activity are performed, similarly to those described in Part 1. If in the event that a patient is unable to return on day 4, the patient may continue dosing with fluticasone propionate twice per day for up to 2 additional days. When the patient returns to the clinic they follow day 4 procedures.

Patients maintain their typical trial activity for a total duration of approximately 2 weeks.

Part 2 of this study runs in parallel with Part 1. Patients who complete Part 2 may participate in Part 1 after a washout period of at least 21 days.

TABLE 2

Schedule of Events for Part 2

| | Screening | | | | Treatment | | Post-treatment | | |
|---|---|---|---|---|---|---|---|---|---|
| | | Induced Sputum & BAC | | | Induced Sputum & BAC | | | | EOS/ ET* |
| Study Procedure | Visit 1 | Visist 2 | Visit 3 | Visit 4 | Visit 5 | Visit 6 | Visit 7 | Visit 8 | Visit 9 |
| Study Day ( [symbol] window days) | -42 to -1 | | | | | 4 (+2) | 5 (+2) | 6 (+2) | 15 (+2) |
| Administrative | | | | | | | | | |
| Informed Consent^{a} | X | | | | | | | | |
| Genomics Sub-study Consent^{b} | X | | | | | | | | |
| Medical History | X | | | | | | | | |
| Demographics | X | | | | | | | | |
| Inclusion Exclusion | X | | | | | | | | |
| Study Drug | | | | | | | | | |
| Study Drug Administration* | | | | | X [symbol] | X^{e.l} | | | |
| Laboratory Testing (all baseline samples collected pre-dose)^{d} | | | | | | | | | |
| Hepatitis (HBsAg, HBcAb, HC′V), and HIV Screen, TB blood test [symbol] | X | | | | | | | | |
| Urine/blood drug Screen (as per site SOP) [symbol] | X | | | | | | | | |
| Hemutology Panel [symbol] | X | | X | | | | X | | X |
| Laboratory Chemistry (including ionized calcium) [symbol] | X | | X | | | | X | | X |
| Serum pregnancy test [symbol] | X | | | | | | | | X |
| FSH [symbol] | X | | | | | | | | |
| Urinalysis (safety labs) | X | | X | | | | X | | X |
| Vital Signs^{e} | Χ [symbol] | X | X [symbol] | X | X [symbol] | | X | X | X |
| Physical Examination | X | | | | | | | | |

[symbol]

indicates text missing or illegible when filed

| | Screening | | | | Treatment | | Post-treatment | | |
|---|---|---|---|---|---|---|---|---|---|
| | | Induced Sputum & BAC | | | lduced Sputum & BAC | | | | EOS/ ET* |
| Study Procedure | Visit 1 | Visit 2 | Visit 3 | Visit 4 | Visit 5 | Visit 6 | Visit 7 | Visit 8 | Visit 9 |
| Study Day ( [symbol] window days) | -42 to -1 | | | | 1 | 4 (-2) | 5 (42) | 6 (+2) | 15 (+2) |
| Weight | X | | | | | | | | X |
| Height | X | | | | | | | | |
| Electrocardiogram [symbol] | X | | X | | x¹² | | X | | X |
| Adverse Events | X | X | X | X | X | | X | X | X |
| Concomitant Medications | X | X | X | X | X | | X | X | X |
| Procedures | | | | | | | | | |
| HDM Skin Prick Test (standard & skin sensitivity test) | X | | | | | | | | |
| In-clinic Spirometry [symbol] | X [symbol] | X [symbol] | X [symbol] | X [symbol] | X [symbol] | X [symbol] | X [symbol] | X [symbol] | X [symbol] |
| FeNO [symbol] | X [symbol] | X [symbol] | X [symbol] | X [symbol] | X [symbol] | X [symbol] | X [symbol] | X [symbol] | X [symbol] |
| Methacholine Challenge | X [symbol] | | | | | | | | |
| Induced Sputum³ | | X [symbol] | X [symbol] | X [symbol] | X [symbol] | X [symbol] | X [symbol] | | |
| BAC with HDM [symbol] | | | X | | | X | | | |
| Sample Collection and Biomarkers | | | | | | | | | |
| DNA (optional)^{k} | | | | | X | | | | |
| Whole blood RNA sample (optional) [symbol] | | | X | X | X | X | X | | X |
| Serum/plasma sST2, calcitonin, MMP12, TARC, PARC, entaxin-3 [symbol] | X | | X [symbol] | X [symbol] | | X [symbol] | X [symbol] | X | |
| Serum total IL-33 [symbol] | X | X | X [symbol] | X | | X [symbol] | X [symbol] | X | X |
| Future biomedical research serum and | X | | | | | | X | | |

[symbol]

indicates text missing or illegible when filed

| | Screening | | | | Treatment | | Post-treatment | | |
|---|---|---|---|---|---|---|---|---|---|
| | | Induced Sputum & BAC | | | Induced Sputum & BAC | | | | EOS/ ET* |
| Study Procedure | Visit 1 | Visit 2 | Visit 3 | Visit 4 | Visit 6 | Visit 6 | Visit 7 | Visit 8 | Visit 9 |
| Study Day (±window days) | -42 to -1 | | | | 1 | 4 (+2) | 5 (+2) | 6 (* 2) | 15 (+2) |
| plasma samplesⁿ | | | | | | | | | |

*EOS/ET: End of Study/ Early Termination: BAC: Bronchial allergen challenge: HDM: House dust mile

### Footnotes for the Schedule of Events Table 2

a. Informed consent may be obtained on a separate visit prior to the screening period.
b. Patients must sign a separate informed consent form (ICF) prior to collection of samples for DNA analysis. Patients who meet the inclusion/exclusion criteria are eligible to enroll in the study regardless of whether they choose to participate in the genomics sub-study.
c. Patients begin self-administration of inhaled fluticasone propionate twice daily for 4 consecutive days, beginning on day 1. Patients receive fluticasone propionate in clinic on day 1 and at home on days 2 and 3. On day 4, patients receive fluticasone propionate (in the clinic) prior to BAC procedures and patient self-administers the second dose that day after sputum induction (either in the clinic or at home).
d. All blood samples (safety laboratory, biomarker, and future biomedical research) should be collected after an overnight fast of at least 8 hours.
e. Details on vital signs measurements:
   el. Vital signs should be collected prior to any blood draw.
   e2. On study day 1, vital signs are collected prior to administration of study drug, immediately (within 10 minutes), and 1, 2, 4, 8, and 12 hours after the end fluticasone administration.
f. Details on ECG measurements:
   f1. Electrocardiogram is performed before blood is drawn.
   f2. On day 1, ECG should be performed prior to and immediately (within 10 minutes) after the end of administration of fluticasone propionate.
g. In-clinic spirometry is performed in accordance with American Thoracic Society criteria standards of acceptability quality control with a standard spirometer (Miller et al. Standardisation of spirometry. Eur Respir J. 2005. 26:319-338.).
h. FeNO should be conducted following a fast of at least 1 hour.
   h1. FeNO measurement must be performed before spirometry and methacholine challenge.
   h2. FeNO measurement must be performed before sputum induction.
   h3. FeNO is measured prior to BAC and at 8 hours and 23 hours post-BAC on BAC days.
   h4. FeNO measurement on day 1 should be pre-dose.
i. For the screening and treatment periods, all pre-BAC sputum samples must be collected 72 hours prior to the BAC.
   i1. During screening, pre-BAC induced sputum sample is collected at least 72 hours before the BAC. If a pre-BAC sample cannot be collected on a first attempt at screening, a repeat may be performed at least 72 hours later. On visit 3, sputum is collected at 8 hours after the challenge, and on the following day, approximately 24 hours after the BAC (visit 4).
   i2. Induced sputum samples (pre-BAC) are collected after the second dose of fluticasone on day 1. A repeat of the pre-BAC sputum sample is not permitted for the on treatment BAC.
   i3. On day 4, patients are given a BAC with HDM similar to that in study Part 1 after receiving a first dose of fluticasone. Induced sputum samples are collected 8 hours and 24 hours after BAC on day 4.
k. DNA sample should be collected on day 1, but may be collected at any visit during the course of the trial.
l. Whole blood RNA samples should only be collected at visits indicated in the table prior to BAC, spirometry, or induced sputum procedures on that day. Whole blood RNA samples should be collected prior to drug administration.
m. Biomarker samples may be collected at times during the study, as specified in the study chart, with the following notes:
   m1. Biomarkers and total IL-33 samples on BAC days should be collected pre-BAC and 8 hours post-BAC, after the 8 hour post-BAC sputum sample was collected.
   m2. Biomarkers collected on the day following BAC should be collected 24 hours after BAC, after the collection of sputum sample

### Study Population

The patient population in this study is adult asthmatics who are allergic to HDM allergen. Numerous studies have demonstrated that BAC induces a marked and reproducible upregulation of inflammatory gene signatures, which may be measured in the induced sputum of allergic asthma patients. Bronchial allergen challenge (BAC) provides an opportunity to assess a potential impact of REGN3500 and dupilumab on lung inflammation. When conducted by experienced investigators in the appropriate setting, BAC is safe and well-tolerated in the patient population proposed for this study. However, because there is a risk of inducing severe, acute bronchoconstriction or anaphylaxis (Diamant et al. Inhaled allergen bronchoprovocation tests. J Allergy Clin Immunol. 2013. 132:1045-1055 e1046), BAC will not be performed on patients with severe or unstable asthma.

Demographic and baseline characteristics include standard demography (e.g., age, gender, race, ethnicity, weight, height), disease characteristics, including medical history and medication history for each patient, and biomarkers (total IL-33, sST2, calcitonin, and MMP12).

Enrollment includes up to 38 patients in the UK for the 2 parts of the study (approximately 32 patients for Part 1, and approximately 6 patients for Part 2).

Up to approximately 38 non-smoking adult patients (male and female), 18 to 60 years of age, with mild persistent allergic asthma are enrolled in this study. Patients included in this study should be clinically stable and only require short-acting β2 agonist on a per-needed basis to control asthma symptoms. Patients included in this study should be allergic to HDM as determined by a skin prick test.

### Study Cohorts

There are 2 parts in this study. Patients in Part 1 of the study constitute Cohort 1, and patients in Part 2 constitute Cohort 2.

In Part 1, approximately 32 patients are randomized 1:1:1:1 to receive REGN3500 (IV single dose), dupilumab (SC 2 doses, Q2W), combination REGN3500 (IV single dose) plus dupilumab (SC 2 doses, Q2W), or placebo (IV single dose and SC 2 doses, Q2W), with approximately 8 patients per treatment group. Approximately 8 patients are assigned to a treatment group in order to obtain data for 6 patients per treatment group in Cohort 1.

In Part 2, approximately 6 patients are treated open-label with inhaled fluticasone propionate in Cohort 2.

### Inclusion Criteria

A patient must meet the following criteria to be eligible for inclusion in the study: 1. Male or female aged between 18 and 60 years inclusive. 2. Has a body mass index (BMI) of 17 to 33 kg/m2 at pre-study (BMI= weight [kg]/ height [m]2) at screening. 3. Has a history of mild allergic asthma for at least 6 months with typical symptoms including cough, wheezing, requiring only treatment with short acting β2 agonist on an as needed basis and is clinically stable. Patient, aside from history of asthma, is judged to be in good health based on medical history, physical examination, vital sign measurements, ECG, and laboratory safety tests performed at the screening and/or prior administration of initial dose of study drug. 4. Has pre-bronchodilator forced expiratory volume FEV1 at screening that is ≥70% of predicted. 5. Is a non-smoker or ex-smoker for at least 12 months. Patient must have less than or equal to a cumulative tobacco exposure of 5 pack-years, where each pack-year = (number of cigarettes smoked per day x number of years of smoking) ÷ 20. 6. Demonstrates atopy to HDM, confirmed by a positive skin-prick test (wheal should be 3 mm or greater than the negative control to define a positive reaction) at screening. 7. Is a dual responder to inhaled BAC as manifested by positive allergen-induced early and late airway bronchoconstriction wherein EAR is defined as a fall in FEV1 of at least 20% from pre-challenge post diluent baseline values during the 30 minutes after inhaled BAC and LAR is defined by a fall from post diluent value of FEV1 of 215% on at least 3 occasions, 2 of which must be consecutive, between 3 to 8 hours following administration of the final concentration of allergen. 8. Is able to tolerate sputum induction and produce adequate sputum after BAC during the screening period (either at the 8-hour or 24-hour post-BAC time points). 9. Understands the alternative treatments available and the risks associated with the study, is able to perform the study procedures, and voluntarily agrees to participate by giving written informed consent. 10. Is willing to comply with the restrictions specified in the study protocol, including the prohibited medications and procedures. 11. Demonstrates a positive response to methacholine challenge test at screening (a fall in FEV1 220% compared to the post-diluent value with methacholine PC20 ≤16 mg/mL).

### Exclusion Criteria

1. Has a history of life-threatening asthma, defined as an asthma episode that required intubation and/or was associated with hypercapnia, respiratory arrest and/or hypoxic seizures. 2. Has been hospitalized or has attended the emergency room for asthma in the 12 months prior to screening. 3. Has had asthma exacerbations or respiratory tract infections within 4 weeks prior to screening or prior to administration of initial dose of study drug. 4. Has diagnosis of other airway/ pulmonary diseases such as Chronic Obstructive Pulmonary Disease (COPD) as defined by the Global Initiative for Chronic Obstructive Lung Disease (GOLD) guidelines (updated 2013), a history of cystic fibrosis, bronchiectasis or alpha-1 antitrypsin deficiency or restrictive lung disease. 5. 5. After screening BAC, patient has a decline in FEV1 below 25% of their predicted and/or FEV1 <1.4 L or has a symptomatic drop in FEV1 associated with shortness of breath unresolved with bronchodilators within a reasonable timeframe (approximately 30 minutes) after allergen exposure. 6. Has a history of severe allergies or history of an anaphylactic reaction or significant intolerability to prescription or non-prescription drugs or food. 7. Has taken oral or systemic corticosteroids within 8 weeks or inhaled corticosteroids/nasal corticosteroids within 4 weeks of screening and/or prior to randomization. 8. Has used any other asthma medications, aside from short acting β-agonists (e.g., leukotrienes receptor antagonist, muscarinic antagonist, terbutaline, theophylline beta blockers, digoxin, NSAIDs, MAO inhibitors or tricyclic antidepressants), within 4 weeks of screening or prior to administration of initial dose of study drug. For approved drugs or investigational biologic agents (e.g., anti-IgE or anti-IL5), has used within 6 months of screening or at least 5 half-lives, whichever is greater. 9. Has had treatment with an investigational drug within 8 weeks or within 5 half-lives (if known), whichever is longer, prior to screening or prior to administration of initial dose of study drug. 10. Has received treatment with a live (attenuated) vaccine within 12 weeks prior to screening. 11. Has an estimated glomerular filtration rate (eGFR) of <60 mL/min/1.73 m² at screening, based on MDRD equation. 12. Is positive for HBsAg, HBcAb, or HCV at screening. 13. Has a known history of human immunodeficiency virus (HIV) infection or HIV seropositivity at the screening visit. 14. Has a positive result in a blood test for tuberculosis (TB) at screening. 15. Has a history of tuberculosis or systemic fungal diseases. 16. Has a diagnosed current or recent (within previous 2 months of screening) bacterial, protozoal, viral, parasitic infection; is suspected of or is at high risk of having a parasitic infection. 17. Has a history of clinically significant neurologic, endocrine, gastrointestinal, cardiovascular, hematological, hepatic, immunological, renal, or any other organ system disease (aside from asthma or other mild allergic disease such as allergic rhinitis). Patients with a history of uncomplicated nephrolithiasis (kidney stones) may be enrolled in the study at the discretion of the investigator. 18. Has had major surgery in the last 2 months or is anticipating surgery during the study or follow-up period. 19. History of cancer, with the exceptions of: patients with adequately treated basal cell carcinoma or carcinoma in situ of the cervix and patients with other malignancies that have been successfully treated for >10 years prior to screening where, in the judgement of both the investigator and the treating physician, appropriate follow-up has revealed no evidence of recurrence through time of screening. 20. Has positive urine drug test results during screening or prior to randomization (e.g., amphetamines/methamphetamines, barbiturates, benzodiazepines, cannabinoids, cocaine, opiates, and cotinine), unless in the opinion of the investigator, the positive test results may be due to the patient’s current permitted medications. 21. Has a history of drug or alcohol abuse within a year prior to the screening visit. 22. Has consumed alcohol within the 48-hour period prior to the screening. 23. Is unwilling to comply with the study’s restrictions on alcohol consumption. During the study, the use of alcohol by adults will be limited to no more than 2 drinks per day (1 drink being equivalent to 12 ounces regular beer, 5 ounces wine, or 1.5 ounces of 80 proof distilled spirits). Patient must refrain from alcohol consumption within 48 hours of a study visit involving a BAC. 24. Is unwilling to comply with the study’s restrictions on caffeine consumption. Patients must refrain from caffeine for 8 hours before all study visits. Decaffeinated products are permitted. 25. Is under the age of legal consent or is mentally or legally incapacitated. 26. Has history of any illness, which, in the opinion of the study investigator, might confound the results of the study or in which participation in the study would pose additional risks to the patient. 27. Has a new exercise routine or a major change to a previous exercise routine within 4 weeks prior to the screening visit. Patients who are unwilling to maintain a similar level of exercise for the duration of the study or to refrain from unusually strenuous exercise for the duration of the trial will be excluded. 28. Is pregnant, or intends to become pregnant, or is lactating. 29. Is a sexually active woman of childbearing potential who is unwilling to practice highly effective contraception prior to the start of the first treatment, during the study, and for at least 4 months after the last dose. Highly effective contraceptive measures include stable use of oral contraceptives associated with inhibition of ovulation (such as contraceptives containing estrogen/progesterone or high dose progesterone) for 2 or more menstrual cycles prior to screening; intrauterine device; intrauterine hormone releasing system; bilateral tubal ligation; vasectomized partner; and or sexual abstinence. Contraception for male patients is not required. 30. Has a known sensitivity to doxycycline and/or tetracycline or to any of the components of the investigational product formulation.

### Study Treatments

### Part 1

REGN3500 is supplied as a lyophilized powder. Placebo that matches REGN3500 (REGN3500-matching placebo) is prepared in the same formulation as REGN3500 but without the addition of protein (i.e., active substance, anti-IL-33 monoclonal antibody). Vials of REGN3500 or REGN3500-matching placebo are reconstituted with sterile water prior to infusion. REGN3500 and REGN3500-matching placebo are administered IV by the investigator, or other qualified study personnel on day 1.

Dupilumab is supplied in pre-filled syringes, each of which can deliver 2 mL of a 150 mg/mL solution (300 mg) of study drug. Placebo that matches dupilumab (dupilumab-matching placebo) is prepared in the same formulation as dupilumab, without the addition of protein. Dupilumab or dupilumab-matching placebo is administered SC by the investigator, or other qualified study personnel, in 2 injections on day 1 (600 mg) and in 1 injection (300 mg) on day 15. All SC injections are in the abdomen.

Patients are randomly assigned evenly to receive REGN3500, dupilumab, REGN3500 plus dupilumab combination, or placebo in one of the following treatment regimens: REGN3500: REGN3500 IV 10 mg/kg plus 2 injections of dupilumab-matching placebo SC on day 1; one injection of dupilumab-matching placebo SC on day 15; Dupilumab: REGN3500-matching placebo for IV plus 2 injections of dupilumab SC 300 mg (600 mg total loading dose) on day 1; one injection of dupilumab SC 300 mg on day 15; REGN3500 Plus Dupilumab Combination: REGN3500 IV 10 mg/kg plus 2 injections of dupilumab SC 300 mg (600 mg total loading dose) on day 1; one injection dupilumab SC 300 mg on day 15; and Placebo: REGN3500-matching placebo IV plus 2 injections of dupilumab-matching placebo SC on day 1; one injection of dupilumab-matching placebo SC on day 15. All patients receive the IV infusion first, followed by the SC injection. Patients are observed for a minimum of 1 hour between the SC injection and the IV infusion.

### Part 2

Fluticasone propionate (250 µg/puff) is supplied in metered dose inhalers and administered by inhalation of 500 µg (2 puffs of 250 µg) per dose twice daily on day 1 through day 4.

### Treatment Assignment

In Part 1 of the study, randomization occurs in 2 separate phases. Eight patients are randomized 1:1:1:1 to receive REGN3500, dupilumab, REGN3500 plus dupilumab combination, or placebo according to a central randomization scheme provided by an interactive voice response system (IVRS)/interactive web response system (IWRS) to the designated study pharmacist (or qualified designee). After the first 8 patients have completed safety assessments through the day 24 (visit 7) study visit and the RSOC has reviewed the safety data and approved further enrollment, another approximately 24 patients may be randomized 1:1:1:1 to receive REGN3500, dupilumab, REGN3500 plus dupilumab combination, or placebo similarly to the first 8 patients.

In Part 2 of the study, approximately 6 patients are enrolled and treated open-label with fluticasone propionate. Patients who complete Part 2 may participate in Part 1 after a washout period of at least 21 days.

For Part 1, study patients, the principal investigators, and study site personnel will remain blinded to all randomization assignments throughout the study. The Regeneron Study Director, Medical Monitor, Study Monitor, and any other Regeneron and contract research organization (CRO) personnel who are in regular contact with the study site remain blinded to all patient randomization assignments.

Selected individuals not involved in the conduct of the study may have access to unblinded data as needed for safety review or other data review.

Blinded study drug kits coded with a medication numbering system are used. In order to maintain the blind, lists linking these codes with product lot numbers are not accessible to individuals involved in study conduct.

Anti-drug antibody and drug concentration results are not communicated to the sites, and the sponsor operational team does not have access to results associated with patient identification until after the final database lock. The bioanalytical analyst, bioanalytical team representative, and clinical pharmacology representative responsible for determining serum drug concentration levels, ADAs, and biomarkers is not blinded to the dosing information.

### Treatment Logistics and Accountability

For Part 1 of the study, a medication numbering system is used to label blinded investigational study drug. Lists linking medication numbers with product lot numbers are maintained by the groups (or companies) responsible for study drug packaging. In order to maintain the blind, these lists are not accessible to individuals involved in the conduct of the study. This automated system is also used to manage the expiration dating of the investigational study drugs, in accordance with the EMA Reflection paper on the use of interactive response technologies (IVRS) in clinical trials, with particular emphasis on the handling of expiration date (EMA/INS/GCP/600788/2011, December 2013). Dupilumab drug labels will include the expiration dates. Expiration dates are not included on drug labels for REGN3500.

For Part 2, open-label study drug displays the product lot number and expiration date on the label. Study drug will be stored at the site at a temperature of 2° C. to 8° C.

REGN3500, dupilumab, and the matching placebo for each study drug are shipped at a temperature of 2° C. to 8° C. to the investigator or designee at regular intervals or as needed during the study. At specified time points during the study (e.g., interim site monitoring visits), at the site close-out visit, and following drug reconciliation and documentation by the site monitor, all opened and unopened study drug are destroyed or returned to the sponsor or designee.

All drug accountability records must be kept current. The investigator must be able to account for all opened and unopened study drug. These records should contain the dates, quantity, and study medication that was: dispensed to each patient, returned from each patient (if applicable), and disposed of at the site or returned to the sponsor or designee. All accountability records must be made available for inspection by the sponsor and regulatory agency inspectors; photocopies must be provided to the sponsor at the conclusion of the study.

All drug compliance records must be kept current and made available for inspection by the sponsor and regulatory agency inspectors.

### Concomitant Medications and Procedures

Any treatment administered from the time of informed consent to the end of the treatment period is considered concomitant medication. This includes medications that were started before the study and are ongoing during the study. Any concomitant medications must be reviewed and approved by the Regeneron Medical Monitor. Information on concomitant medication for each patient is recorded at each study visit from screening through the end of study.

Initiation of treatment with any new prescription medication is prohibited from the time of screening until the end of study visit, unless agreed to by the principal investigator or designee and the medical monitor.

In addition, per the eligibility criteria, the following medications and procedures are prohibited during the study: leukotriene receptor antagonist, muscarinic antagonist, terbutaline, theophylline, beta blockers, digoxin, NSAIDs, MAO inhibitors, or tricyclic antidepressants, Roflumilast, and cromoglycate; biologic therapy (such as an anti-IgE, anti-IL-5) or immunotherapy (subcutaneous immunotherapy (SCIT), sublingual immunotherapy (SLIT), or oral immunotherapy (OIT)); treatment with a live (attenuated) vaccine; oral and systemic corticosteroids; treatment with an inhaled or nasal corticosteroid; onset of new exercise routine or major change to a previous exercise routine within 4 weeks prior to the screening visit (patients must have been willing to maintain a similar level of exercise for the duration of the study and refrain from unusually strenuous exercise for the duration of the trial); during the study, the use of alcohol by adults will be limited to no more than 2 drinks per day (1 drink being equivalent to 12 ounces regular beer, 5 ounces wine, or 1.5 ounces of 80 proof distilled spirits) and patients must refrain from alcohol consumption for the 48-hour period prior to the screening and on-allergen study visit; and patients must refrain from caffeine for 8 hours before all study visits (decaffeinated products are permitted.)

The following medications are permitted: short acting β-agonists; thyroid replacement therapy by patients who have been on stable doses for >6 months prior to screening; vitamins and calcium supplements; over-the-counter antihistamines and decongestants; paracetamol (care should be taken to adhere to all guidance related to paracetamol administration and to not exceed local maximally allowed daily doses); laxatives; antacids; and heat-killed vaccine.

### Study Procedures

The following procedures are performed for the sole purpose of determining study eligibility or characterizing the baseline population: demographics, medical history, HIV, hepatitis and drug screening, HDM skin prick tests, and methacholine challenge.

Safety Procedures: patient safety is monitored via AEs reported by the patients or observed by the Investigator and via clinical laboratory tests (e.g., biochemistry, hematology, and urinalysis), vital signs, and standard 12-lead ECG automatic reading. Clinically significant abnormalities (if any) are monitored until resolution or until clinically stable.

Vital Signs: vital signs, including temperature, blood pressure, pulse, and respiration, are collected, after at least 5 minutes of rest, pre-dose at time points according to Table 1 and Table 2.

Physical Examination: a thorough and complete physical examination, including height and weight, is performed at time points according to Table 1 and Table 2. Care should be taken to examine and assess any abnormalities that may be present, as indicated by patient’s medical history.

Electrocardiogram: electrocardiograms should be performed before blood is drawn during visits requiring blood draws. A standard 12-lead ECG is performed at the time points specified in Table 1 and Table 2. Heart rate is recorded from the ventricular rate, and the PR, QRS, RR, and QT intervals are recorded. The ECG strips or reports are retained with the source. For the ECG procedure, 12-lead ECGs are digitally recorded systematically after the patient has been in the supine position for at least 10 minutes. The electrodes are positioned in the same location for each ECG recording throughout the study. Each ECG consists of a 10-second recording of the 12 leads simultaneously, leading to a single 12 lead ECG (25 mm/s, 10 mm/mV) printout with evaluation (HR, PR, QRS, RR, QT intervals, and QTc) including date, time, initials and number of the patient, signature of the research physician, and at least 3 complexes for each lead. The reading is used for immediate safety assessment. The investigator’s medical opinion and ECG values are recorded in the eCRF.

HDM Skin Prick Tests: at the screening visit, a standard skin prick test using HDM allergen is performed to confirm inclusion criteria. Following this, a serial skin prick test using diluted HDM solutions is performed to determine skin sensitivity. The skin sensitivity is used in determining the allergen dose regimen to be used during the screening BAC.

Methacholine Challenge: at the screening visit, a methacholine challenge is performed to confirm inclusion criteria and determine the allergen dose regimen used during the screening BAC. The methacholine challenge is performed according to ATS/ERS (1999) guidelines (Crapo RO, et al. Guidelines for methacholine and exercise challenge testing-1999. This official statement of the American Thoracic Society was adopted by the ATS Board of Directors, July 1999. Am J Respir Crit Care Med. 2000 Jan;161(1):309-29), using a 2-minute tidal breathing protocol.

### Laboratory Testing

Hematology, chemistry, urinalysis, and pregnancy testing samples are analyzed by a central laboratory. Detailed instructions for blood sample collection are in the laboratory manual provided to study sites.

Samples for laboratory testing will be collected at visits according to Table 1. Tests will include:

Blood Chemistry

| | | |
|---|---|---|
| Sodium | Total protein, serum | Total bilirubin |
| Potassium | Creatinine | Total cholesterol^{∗} |
| Chloride | Blood urea nitrogen (BUN) | Triglycerides |
| Carbon dioxide | Aspartate aminotransferase (AST) | Uric acid |
| Calcium | Alanine aminotransferase (ALT) | Creatine phosphokinase (CPK) |
| Glucose (fasting) | Alkaline phosphatase | Ionized Calcium |
| Albumin | Lactate dehydrogenase (LDH) | |

∗(low-density lipoprotein [LDL] and high-density lipoprotein [HDL])

| | |
|---|---|
| Hematology | |
| Hemoglobin | Differential: |
| Hematocrit | Neutrophils |
| Red blood cells (RBCs) | Lymphocytes |
| White blood cells (WBCs) | Monocytes |
| Red cell indices | Basophils |
| Platelet count | Eosinophils |

Urinalysis

| | | |
|---|---|---|
| Color | Glucose | RBC |
| Clarity | Blood | Hyaline and other casts |
| pH | Bilirubin | Bacteria |
| Specific gravity | Leukocyte esterase | Epithelial cells |
| Ketones | Nitrite | Crystals |
| Protein | WBC | Yeast |

### Other Laboratory Tests

Patients are tested for follicle-stimulating hormone (FSH) levels (menopausal women only), and undergo serum and urine pregnancy testing (women only) at time points listed in Table 1 and Table 2. Samples are collected for assessment of serum/plasma sST2, and total IL-33, calcitonin, MMP12, TARC, PARC, eotaxin-3. Tests for HIV, HBsAg, HBcAb, and HCV, and blood test for TB are performed. In addition urine samples are collected for a drug screening. Intact parathyroid hormone (iPTH), 25-hydroxy Vitamin D samples are collected in Part 1 (Table 1) and are not collected in study Part 2.

### Pharmacokinetic and Anti-Drug Antibody Procedures

Samples for drug concentration are collected at time points listed in Table 1. Any unused samples may be used for exploratory biomarker research.

Anti-Drug Antibody Measurements and Samples: samples for ADA assessments are collected at time points listed in Table 1. Results from any exploratory analyses are reported separately from the clinical study report. Unused samples collected for ADA analyses may be used for future biomedical research.

Pharmacodynamic Procedures: pharmacodynamic procedures include BAC, sputum induction, measurement of gene expression levels in sputum mRNA, in-clinic measures of FEV1, FeNO, and measures of circulating biomarkers.

Bronchial Allergen Challenge: the BAC using HDM is performed, as per standard procedures leveraging the Cockcroft allergen calculation (Cockcroft et al. The links between allergen skin test sensitivity, airway responsiveness and airway response to allergen. Allergy. 2005 Jan;60(1):56-59) and as delineated in the study manual to confirm the presence of an early and late phase response at screening visits specified in Table 1 and Table 2. Post-treatment BAC is performed utilizing the dose regimen calculated from the screening BAC. The EAR is measured as the largest decrease in FEV1 within the first 120 minutes (30 minutes for inclusion at screening). The LAR is measured as the decrease in FEV1 at 3 to 8 hours after allergen inhalation.

Sputum Induction: sputum induction is performed at time points according to Table 1 and Table 2. Hypertonic saline (4.5% NaCl) is nebulized and inhaled through the mouth, with the nose held closed with a clip, for 4 periods of 5 minutes. Spirometry is performed approximately 7 minutes after each 5-minute period of induction as a safety procedure. Sputum induction is generally well-tolerated, although some patients develop wheeze and dyspnea. Any airway constriction caused by sputum induction with hypertonic saline may be quickly reversed by treatment with an inhaled short-acting β2 agonist (Wong et al. Safety of one method of sputum induction in asthmatic subjects. Am J Respir Crit Care Med. 1997. 156:299-303.).

Measurements of Gene Expression Levels in Induced Sputum: induced sputum samples are collected at time points according to Table 1 and Table 2. Sputum is processed into RNA and gene expression analyses are performed using either Taqman assays, RNAseq or Nanostring. The genes studied are those believed to be relevant to the pathophysiology of asthma (Type 1 and Type 2 inflammation), target engagement, and mechanism of action of REGN3500, dupilumab, and/or REGN3500 plus dupilumab combination therapy. The molecular signature for Type 1 inflammation may include the genes for IFNγ, CXCL9, CXCL10, CXCL11, IL-8, MPO, and neutrophil elastase. The molecular signature for Type 2 inflammation may include the genes for IL-4, IL-5, IL-13, IL-9, CCL17, CCL26, CCL13, and CCL11. The list of genes studied may be altered or expanded as additional potentially relevant or novel biomarkers are discovered during the study.

Spirometry: In-clinic spirometry is performed in accordance with American Thoracic Society criteria standards of acceptability quality control with a standard spirometer (Miller et al. Force. Standardisation of spirometry. Eur Respir J. 2005. 26:319-338.). During the study (screening and treatment, and post-treatment periods), patients are required to undergo in-clinic spirometry (FEV1) measurement at every scheduled study visit (Table 1 and Table 2).

Fractional Exhaled Nitric Oxide: measurement of FeNO level in asthmatic patients is used as a marker of airway inflammation. Fractional exhaled nitric oxide is analyzed from exhaled breath condensates. Patients are instructed to refrain from eating and drinking nitrate rich foods for at least 2 hours AND any food or drink for at least 1 hour prior to FeNO measurements and FeNO measurements should be made prior to any spirometry. During the study (screening, treatment, and post-treatment periods), patients are required to undergo FeNO measurement as specified (Table 1 and Table 2).

Circulating Biomarkers: circulating biomarker samples are collected at time points according to Table 1 and Table 2. Biomarker measurements are performed in serum and plasma samples to determine effects on biomarkers of inflammatory diseases pathobiology or relevant physiological and pathogenic processes. The biomarkers studied are ones believed to be relevant to the pathophysiology of diseases, target engagement, mechanism of action of REGN3500 (and/or combination therapy) and possible toxicities. Biomarkers studied in the blood may include but need not be limited to, IL-33, soluble ST2, calcitonin, and MMP12.

Future Biomedical Research: the future biomedical research samples, as well as unused PK and ADA samples, will be stored for up to 15 years after the final date of the database lock. The unused samples may be utilized for future biomedical research, including research on inflammatory diseases. After 15 years, any samples that remain will be destroyed.

Genomics Sub-study - Optional: patients who agree to participate in the genomics sub-study are required to sign a separate genomics sub-study ICF before collection of the samples. Patients are not required to participate in the genomics sub-study in order to enroll in the primary study. Both DNA and whole blood RNA samples are covered in the genomics sub-study. Samples for DNA extraction should be collected on day 1/baseline (pre-dose), but may be collected at any study visit. Samples for whole blood RNA should be collected as specified in the study chart. DNA samples for the genomics sub-study are double-coded as defined by the International Council of Harmonisation (ICH) guideline E15. Sub-study samples will be stored for up to 15 years after the final date of the database lock and may be used for research purposes. The purpose of the genomic analyses is to identify genomic associations with clinical or biomarker response, other clinical outcome measures and possible AEs. In addition, associations between genomic variants and prognosis or progression of as well as other diseases may also be studied. These data may be used or combined with data collected from other studies to identify and validate genomic markers related to the study drug or diseases. Analyses may include sequence determination or single nucleotide polymorphism studies of candidate genes and surrounding genomic regions. Other methods, including whole exome sequencing, whole genome sequencing, DNA copy number variation, and transcriptome sequencing may also be utilized. The list of methods may be expanded to include novel methodology that may be developed during the course of this study or sample storage period.

### Endpoints

### Sputum mRNA Measures

Induced sputum samples have been used in clinical studies of asthma to assess airway inflammation. Studies comparing sputum from asthmatics with sputum from normal controls have found elevated concentrations of IL-33 and ST2 (Hamzaoui et al. Induced sputum levels of IL-33 and soluble ST2 in young asthmatic children. J Asthma. 2013. 50:803-809 and Salter et al. IL-25 and IL-33 induce Type 2 inflammation in basophils from subjects with allergic asthma. Respir Res. 2016. 17:5), eotaxin, TARC (Heijink et al. Effect of ciclesonide treatment on allergen-induced changes in T cell regulation in asthma. Int Arch Allergy Immunol. 2008. 145:111-121 and Sekiya et al. Increased levels of a TH2-type CC chemokine thymus and activation-regulated chemokine (TARC) in serum and induced sputum of asthmatics. Allergy. 2002. 57:173-177) and both IL-5 and IL-13 (Park et al. Interleukin-13 and interleukin-5 in induced sputum of eosinophilic bronchitis: comparison with asthma. Chest. 2005. 128:1921-1927 and Peters et al. Measures of gene expression in sputum cells can identify TH2-high and TH2-low subtypes of asthma. J Allergy Clin Immunol. 2014. 133:388-394) with protein and/or RNA. Sputum cytokines such as IL-4, IL-5, and IL-13 are elevated and associated with the presence of asthma symptoms and severity (Truyen et al. Evaluation of airway inflammation by quantitative Th1/Th2 cytokine mRNA measurement in sputum of asthma patients. Thorax. 2006. 61:202-208.)

In previous studies, BAC in mild asthmatic patients acutely increased levels of Type 2 cytokines, such as IL-13 and IL-5 by approximately 10X in the lung. Treatment with inhaled corticosteroids significantly suppressed this BAC-mediated upregulation of protein and mRNA levels of Type 2 cytokines (Zuiker et al. Kinetics of TH2 biomarkers in sputum of asthmatics following inhaled allergen. Eur Clin Respir J. 2015. 2 and Zuiker et al. Sputum RNA signature in allergic asthmatics following allergen bronchoprovocation test. Eur Clin Respir J. 2016. 3:31324).

The endpoints in this study are designed to investigate the effect of REGN3500, dupilumab, and combined REGN3500 plus dupilumab treatment on Type 2 inflammatory gene expression. In addition, sputum mRNA measurements are analyzed to assess a broader gene expression profile, which includes genes involved in type 1 and 2 inflammation and genes that reflect change in cellular content.

### Sputum Cytokines and Chemokines

Previous studies have shown that cytokines and chemokines may be measured in sputum induced after a BAC. Though the effect size of changes seen in previous studies have shown that measurement of mRNA gene signatures may be superior to that of protein signatures, this study collects samples for cytokine and chemokine protein evaluation as an exploratory endpoint. Cytokines and chemokines related to both the IL-33 and the IL4R pathways, including IL-13, IL-5, tumor necrosis factor-alpha (TNFα), TARC, pulmonary and activation-regulated chemokine (PARC), and eotaxin-3, are expected to be elevated after a BAC. This increase in cytokines and chemokines is expected to be blunted by treatment with REGN3500 and/or dupilumab.

### Early and Late Phase Decreased FEV1 After Bronchial Allergen Challenge

Change in pulmonary function after a BAC is a standard endpoint for most allergen challenge studies evaluating effect of inhaled corticosteroids. In sensitized patients, allergen inhalation results in an acute response characterized by bronchoconstriction within 0 to 2 hours after exposure, which is referred to as the early allergen response (EAR). This EAR is thought to represent primarily the release of preformed mast cell mediators and is typically not responsive to steroid. Early allergen response is often followed by a late allergen response (LAR) that occurs approximately 3 to 8 hours after exposure. This LAR is seen in 50% to 60% of adult asthmatic patients but will be required for entry for all patients in this study. The LAR coincides with the initial influx of inflammatory cells and is generally responsive to steroid.

The reproducibility of these FEV1 endpoints is reported to be sufficient to demonstrate an approximately 50% attenuation of the EAR and/or the LAR, with >90% power, if a crossover study design is implemented with 12 patients. For this parallel design study, the goal is to evaluate changes in sputum gene signatures that reflect the change in the inflammatory stimulus, which ultimately lead to changes in FEV1.

### Fractional Exhaled Nitric Oxide Measures

In a BAC, sputum eosinophils have been shown to increase in asthmatic patients who display a late phase response. Although an association of sputum eosinophils and FeNO has been reported, FeNO is not an eosinophil-specific marker and may be present in non-eosinophilic inflammation (Haldar et al. Mepolizumab and exacerbations of refractory eosinophilic asthma. N Engl J Med. 2009. 360:973-984.). A strong correlation between FeNO levels at baseline and REGN3500 and/or dupilumab response, as evaluated by change in mRNA levels of Type 1 and Type 2 genes, will support the utility of FeNO, a relatively simple and inexpensive measure, as a PD biomarker for responsive patients for future studies.

### Statistical Analysis

The study is powered for the primary endpoint, suppression of sputum IL-13, IL-5 and ST2 mRNA for an active treatment (rather than relative to placebo), not change in the LAR, which is historically the primary endpoint of BAC trials (typically ~20 patients per treatment group). Six patients per treatment group provides >99% power at 2-sided 0.05 significance level to detect a change in IL-5 and IL-13 gene expression levels that is equivalent to the change observed with inhaled corticosteroids (maximum observed effect size of 3.7 based on treatment difference over placebo). In order to ensure that sputum sample is produced post-BAC and is adequate for mRNA measures, approximately 8 patients per treatment group may be enrolled in Part 1 of the study.

Efficacy endpoints will be analyzed using the full analysis set (FAS). For part 1 of the study, the FAS includes all randomized patients and is based on the treatment allocated (as randomized).

Safety Analysis Set: the safety analysis set (SAF) includes all randomized patients who received at least 1 dose of study medication. Patients are be analyzed as-treated. All safety analyses are summarized based on the SAF.

Pharmacokinetic Analysis Set: the PK analysis set includes all treated patients who received any study drug and who had at least 1 non-missing post-dose PK results following the administration of study drug.

Anti-Drug Antibody (ADA) Analysis Set: the ADA population includes all treated patients who received any study drug and who had at least 1 non-missing ADA result after first dose of the study drug.

### Statistical Methods

For continuous variables, descriptive statistics include the following information: the number of patients reflected in the calculation (n), mean, median, standard deviation, Q1, Q3, minimum, and maximum. For categorical or ordinal data, frequencies and percentages are displayed for each category.

For patient disposition, the following will be provided: the total number of screened patients: met the inclusion criteria regarding the target indication and signed the ICF; the total number of randomized patients: received a randomization number; the total number of patients in each analysis set; the total number of patients who discontinued the study, and the reasons for discontinuation; a listing of patients treated but not randomized, patients randomized but not treated, and patients randomized but not treated as randomized; and a listing of patients prematurely discontinued from treatment, along with reasons for discontinuation.

Demographic and baseline characteristics are summarized descriptively by treatment group and by all patients combined.

Primary Efficacy Analysis: for each individual patient in the study, a BAC using HDM is performed at screening and at day 29. Fold-change in induced IL-13 mRNA from pre-BAC values is obtained 8 hours and 24 hours after challenge (at screening and day 29). The difference between the fold change from pre-BAC values at screening and fold change from pre-BAC values at day 29 (8 hours and 24 hours) is computed for each patient. These fold changes from baseline between the screening and day 29 visit are compared between the REGN3500, dupilumab, REGN3500 plus dupilumab combination, and placebo groups. Additionally, the effect of REGN3500 on a broader type 2 allergen signature is assessed. Fold-change difference in mRNA expression levels is analyzed separately using analysis of covariance model (ANCOVA). Descriptive statistics are also provided by treatment and time point on mRNA levels and fold changes from baseline.

Secondary Efficacy Analysis: for the gene expression based endpoints, the continuous efficacy variables use the same analysis method as the primary analysis. Pharmacokinetic parameters of REGN3500 are summarized by arithmetic mean, SD, coefficient of variation (CV, in %), minimum, Q1, median, Q3, maximum and number of observations.

Safety Analysis: treatment compliance/administration and all clinical safety variables is analyzed using the SAF. The safety analysis is based on the SAF. This includes reported TEAEs and other safety information (i.e., clinical laboratory evaluations, vital signs, and 12-lead ECG results). A summary of safety results is presented for each treatment group. For safety variables, 3 observation periods are defined: the pretreatment period is defined as the time from signing the ICF to before the first dose of study drug. The treatment period is defined as the day from first dose of study drug to the last dose of study drug + 7 days. The posttreatment period is defined as the time after the last dose of study drug + 7 days. Treatment-emergent adverse events (TEAEs) are defined as those that are not present at baseline or represent the exacerbation of a pre-existing condition during the on-treatment period.

Vital Signs: vital signs (temperature, pulse, blood pressure, and respiration rate) are summarized by baseline and change from baseline to each scheduled assessment time with descriptive statistics.

Laboratory Tests: laboratory test results are summarized by baseline and change from baseline to each scheduled assessment time with descriptive statistics. Number and percentage of patients with a potentially clinically significant value (PCSV) at any post-randomization time point are summarized for each clinical laboratory test. Shift tables based on baseline normal/abnormal and other tabular and graphical methods may be used to present the results for laboratory tests of interest.

Treatment Exposure: the duration of exposure during the study is presented by treatment group (placebo pooled) and calculated as: (date of last study drug injection - date of first drug injection) + 7. The number (%) of patients randomized and exposed to double-blind study drug are presented by specific time periods for each treatment group. In addition, duration of exposure during the study is summarized for each treatment group using number of patients, mean, SD, minimum, Q1, median, Q3, and maximum.

Treatment Compliance: the compliance with protocol-defined investigational product is calculated as follows: treatment Compliance = (number of investigational product injections during exposure period) / (number of planned investigational product injections during exposure period) × 100%. The treatment compliance is presented by specific ranges for each treatment group in Part 1 of the study.

Analysis of Drug Concentration Data: drug and total target concentrations at each sampling time are summarized using descriptive statistics. Summary of drug and total target concentrations is presented by nominal time point (i.e., the time points specified in the protocol). Plots of the concentrations of REGN3500 and total IL-33 are presented over time (linear and log scales). When the scale is linear, concentrations below the lower limit of quantification (LLOQ) are set to zero. In the log-scaled figures, concentrations below the LLOQ are imputed as LLOQ/2.

Analysis of Anti-Drug Antibody Data: incidence of positive response in the ADA assay for REGN3500 and/or dupilumab is assessed as absolute occurrence (n) and percent of patients (%), by group. Listing of all ADA titer levels is provided for patients positive in the ADA assay for REGN3500 and/or dupilumab. Plots of concentrations of functional REGN3500 may be examined and the potential influence of ADA on individual concentration-time profiles may be evaluated. Assessment of the potential impact of ADA for REGN3500 and/or dupilumab on safety and efficacy may be explored.

Analysis of Pharmacodynamic and Biomarker Data: pharmacodynamic effects of REGN3500, dupilumab and REGN3500 + dupilumab on sputum gene expression (mRNA) results are summarized as mean log2 fold-change (FCH) values (SD, Q1, Q2, SEM, minimum, maximum and number of observations) of cytokine gene expression normalized to housekeeping gene expression. Changes in gene expression are evaluated as a change after allergen inhalation challenge as well as change over time from pre-treatment baseline. Increases in cytokine gene expression after allergen inhalation challenges at week 4 and week 8 are compared with pre-dose levels to investigate a potential mitigating effects of REGN3500, dupilumab, or a combination of REGN3500 and dupilumab relative to placebo. Pharmacodynamic effects of REGN3500, dupilumab and a combination of REGN3500 and dupilumab on FEV1 are summarized as measured values (SD, Q1, Q3, SEM, minimum, mean, median, maximum, and number of observations), percentage change from baseline (SD, Q1, Q3, SEM, minimum, mean, median, maximum, and number of observations) at day 29 (in-clinic FEV1). Pharmacodynamic parameters (circulating markers and FeNO) of REGN3500, dupilumab and a combination of REGN3500 and dupilumab are summarized by measured values (SD, Q1, Q3, SEM, minimum, mean, median, maximum, and number of observations), change from baseline (SD, Q1, Q3, SEM, minimum, mean, median, maximum, and number of observations) and percentage change from baseline (SD, Q1, Q3, SEM, minimum, mean, median, maximum, and number of observations). Correlation analyses between baseline concentration of IL-33 and other baseline biomarkers (calcitonin, sST2, and MMP12, TARC, PARC, eotaxin-3 and FeNO) is performed. A scatter plot with Pearson or Spearman correlation is provided for each correlation analysis.

Additional Statistical Data Handling Conventions: definition of baseline. Unless otherwise specified, the baseline assessment for all measurements is the latest available valid measurement taken prior to the administration of the study drug. For most variables, day 1 procedures and assessments are considered to be baseline.

General rules for handling missing data: unless otherwise specified below, missing sampling or concentration values are not imputed, but left missing in the calculation of derived PK parameters. If the actual sampling time is missing, but a valid concentration value has been measured, the scheduled protocol time may be used for the calculation of derived PK parameters. A missing pre-dose value with SC administration is set to 0 for the PK calculations. If the start date of an AE or concomitant medication is incomplete or missing, it will be assumed to have occurred on or after the intake of study medication, except if an incomplete date (e.g., month and year) clearly indicates that the event started prior to treatment. If the partial date indicates the same month or year of the intake of study medication date, then the start date by the study medication intake date will be imputed; otherwise, the missing day or month by the first day or the first month is imputed. No imputations for missing laboratory data, ECG data, vital sign data, or physical examination data are made. Assessments taken outside of protocol allowable windows will be displayed according to the CRF assessment recorded by the investigator. Extra assessments (laboratory data or vital signs associated with non-protocol clinical visits or obtained in the course of investigating or managing AEs) are included in listings, but not summaries. If more than one laboratory value is available for a given visit, the first observation is used in summaries and all observations are presented in listings.

### Results

Treatment with REGN3500 and dupilumab monotherapy significantly inhibited allergen-induced eosinophilic signature and broader type 2 inflammatory signature, including CCL26, CCL17, and SIGLEC8. Many of the genes suppressed by REGN3500 were also suppressed by dupilumab, albeit with different dynamics. Several genes were suppressed in only one of the arms suggesting that the two molecules regulate both overlapping and distinct pathways. The combination of both molecules provided suppression of type 2 inflammation similar to that observed with monotherapies.

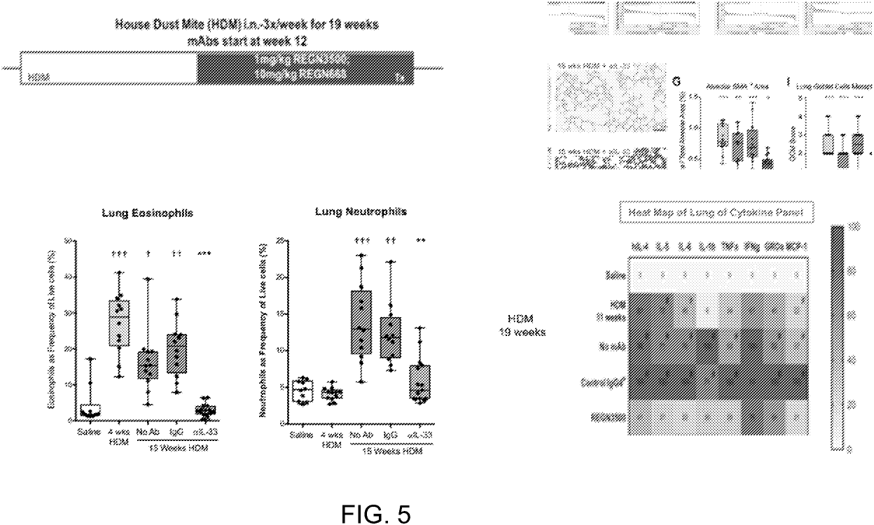

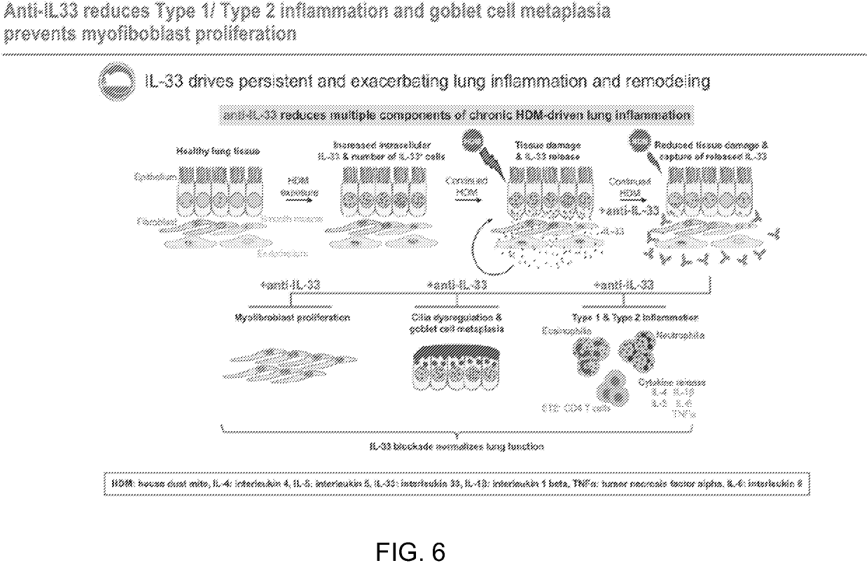

FIG. 5 presents data showing that treatment with REGN3500 reduced inflammation in a chronic house dust mite (HDM) model of lung inflammation. Treatment with REGN3 500 suppressed pro-inflammatory cytokines and chemokines. These results were based on data showing levels of lung eosinophils and lung neutrophils in the HDM model, with and without anti-IL-33 treatment. Other data presented includes a heat map of lung of cytokine panel showing levels of hIL-4, IL-5, IL-1b, TNFα, IFNg, GROa, and MCP-1. Alveolar SMA testing was also performed.

An analysis of the bronchial allergen challenge molecular signature in sputum is shown in FIG. 8, which depicts the expression of various genes related to type 2 inflammation before allergen challenge, 8 hours after the allergen challenge, and 24 hours after the allergen challenge. The top genes induced by the bronchial allergen challenge at screening were enriched for type 2 inflammation, including IL-4, IL-5, IL-13, IL-9, IL1RL1 (IL-33 receptor), Eot-3 (CCL26), TARC (CCL17), and FCER2.

An analysis of the suppression by REGN3500 of various genes induced by the allergen challenge is shown in FIG. 9, which shows that various genes of type 2 inflammatory cytokines and chemokines that that were induced in the bronchial allergen challenge were suppressed by REGN3500, including IL-5, IL-13, Eot-3 (CCL26), and TARC (CCL17). Other genes suppressed by REGN3500 and induced by the bronchial allergen challenge included CCL1 (a ligand for CCR8 that attracts activated Th2 type and Treg cells), CCL26, FCER2, SIGLEC8, and CCL17.

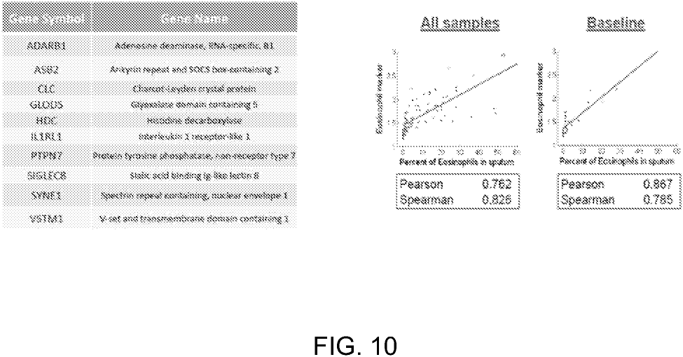

The gene signature utilized to evaluate treatment effects on sputum eosinophils is presented in FIG. 10. A set of 10 genes showed high correlation to eosinophils counts in sputum, both pre- and post-allergen challenge. These genes included ADARB1, ASB2, CLC, GLOD5, HDC, IL1RL1, PTPN7, SIGLEC8, SYNE1, and VSTM1. These genes are not exclusive to eosinophils, e.g., SIGLEC8 is expressed in eosinophils, basophils, and mast cells; HDC is expressed in mast cells; and VSTM1 is expressed in myeloid cells.

FIG. 11 shows that REGN3500 treatment suppressed eosinophil signature genes in sputum. Data is presented for ADARB1, ASB2, CLC, HDC, IL1RL1, PTPN7, SIGLEC8, SYNE1, and VSTM1. No anti-IL-33 (REGN3500) treatment-mediated effects were noted on neutrophil signature genes.

FIG. 12 shows that REGN3500 treatment suppressed type 2 inflammatory signature genes in sputum. Data is presented for IL-4, IL-13, CCL26, CCL13, CCL17, CCL11, POSTN, IL-5, and IL-9. FIG. 12 also shows that type 1 inflammatory signature genes were not induced by the allergen challenge.

Collectively, the data of FIGS. 8-12 show that the reduction in blood eosinophils was a consistent pharmacodynamic effect of anti-IL-33. No anti-IL-33 mediated reduction in neutrophils was observed. Further, no anti-IL-33 mediated reduction in other circulating type 2 inflammatory mediators was observed.

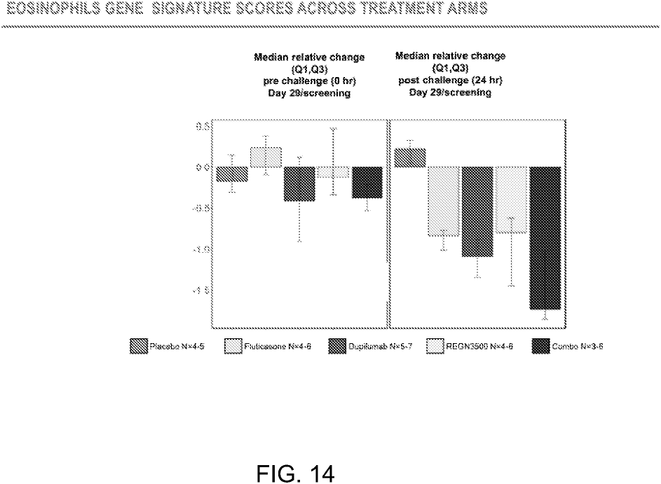

FIG. 14 shows that both dupilumab and REGN3500 were able to reduce eosinophil gene signature scores post bronchial allergen challenge. The combination treatment of dupilumab and REGN3500 was the most effective treatment in reducing eosinophil gene signature scores post bronchial allergen challenge. FIG. 14 depicts eosinophil gene signature scores across treatment arms. The arms include placebo, fluticasone, dupilumab, REGN3500, and the combination therapy of dupilumab and REGN3500. Results are presented pre and post bronchial allergen challenge.

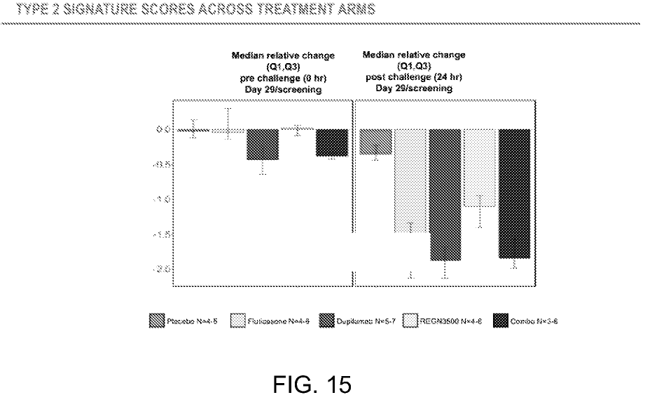

FIG. 15 shows a lower decrease of type 2 signature scores in the REGN3500 treatment arm than the fluticasone treatment arm. FIG. 15 depicts type 2 signature scores across the treatment arms. The arms include placebo, fluticasone, dupilumab, REGN3500, and the combination therapy of dupilumab and REGN3500. Results are presented pre- and post-bronchial allergen challenge.

FIG. 16 presents data to show the genes affected by the various treatment arms at 8 and 24 hours post bronchial allergen challenge. FIG. 16 depicts genes effected by placebo, fluticasone, dupilumab, REGN3500, and the combination therapy of dupilumab and REGN3500. Results are presented at screening and at treatment which occurs post bronchial allergen challenge. Genes tested include, from top to bottom, BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, P1K3R6, LTC4S, CLEC11A, TRABD2A, DLGAP3, VDR, DKFZp686M11215, SIGLEC12, BC016361, BC052769, and RHOH.

FIG. 17 shows, from top to bottom, that the top genes induced by the bronchial allergen challenge at 24 hours and suppressed by REGN3500 were ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS, and RD3. FIG. 17 depicts genes effected by placebo, fluticasone, dupilumab, REGN3500, and the combination therapy of dupilumab and REGN3500. Results are presented at screening and at treatment which occurs post bronchial allergen challenge.

What is claimed is:

1. A method for treating allergic asthma in a subject in need thereof comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for treating allergic asthma in a subject in need thereof comprising administering to the subject an antibody or antigen-binding fragment thereof that specifically binds interleukin-4R (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

2. The method of claim 1, wherein the antibody or antigen-binding fragment thereof that specifically binds interleukin-33 (IL-33):
(a) comprises a heavy chain variable region (HCVR) comprising the amino acid sequence of SEQ ID NO: 2 and a light chain variable region (LCVR) comprising the amino acid sequence of SEQ ID NO: 10; or
(b) comprises a heavy chain comprising the amino acid sequence of SEQ ID NO: 18 and a light chain comprising the amino acid sequence of SEQ ID NO: 20; or
(c) is administered intravenously at a dose of 10 mg/kg; or
(d) is administered subcutaneously at a dose of about 0.1 mg to about 600 mg, about 100 mg to about 400 mg, or about 300 mg; or
(e) is administered subcutaneously at an initial dose of about 600 mg or about 300 mg; or
(f) is administered subcutaneously in one or more secondary doses of about 300 mg; or
(g) is administered every week (q1w), every other week (q2w), every three weeks (q3w), or every four weeks (q4w); or
(h) is administered every other week (q2w); or
(i) is administered subcutaneously; or
(j) is administered subcutaneously using an autoinjector, a needle and syringe, or a pen delivery device.

3-8. (canceled)

9. The method of claim 1, wherein the antibody or antigen-binding fragment thereof that specifically binds interleukin-4R (IL-4R):
(a) comprises a heavy chain variable region (HCVR) comprising the amino acid sequence of SEQ ID NO: 27 and a light chain variable region (LCVR) comprising the amino acid sequence of SEQ ID NO: 28; or
(b) comprises dupilumab; or
(c) is administered at a dose of about 0.1 mg to about 600 mg, about 100 mg to about 400 mg, or about 300 mg; or
(d) is administered at an initial dose of about 600 mg; or
(e) is administered in one or more secondary doses of about 300 mg; or
(f) is administered every week (q1w), every other week (q2w), every three weeks (q3w), or every four weeks (q4w); or
(g) is administered every other week (q2w); or
(h) is administered subcutaneously; or
(i) is administered subcutaneously using an autoinjector, a needle and syringe, or a pen delivery device.

10-19. (canceled)

20. A method for treating allergic asthma in a subject in need thereof comprising administering to the subject:
a first antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; and
a second antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

21. The method of claim 20, wherein:
(a) the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10; or
(b) the first antibody or antigen-binding fragment thereof comprises a heavy chain comprising the amino acid sequence of SEQ ID NO: 18 and a light chain comprising the amino acid sequence of SEQ ID NO: 20; or
(c) the second antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28; or
(d) the second antibody or antigen-binding fragment thereof comprises dupilumab.

22-24. (canceled)

25. The method of claim 20, wherein:
(a) the second antibody or antigen binding fragment thereof is administered at a dose of about 0.1 mg to about 600 mg, about 100 mg to about 400 mg, or about 300 mg, or
(b) the second antibody or antigen binding fragment thereof is administered at an initial dose of about 600 mg; or
(c) the second antibody or antigen binding fragment thereof is administered in one or more subsequent doses of about 300 mg of the antibody or antigen binding fragment thereof; or
(d) the second antibody or antigen-binding fragment thereof is administered every week (q1w), every other week (q2w), once every three weeks (q3w), or once every four weeks (q4w); or
(e) the second antibody or antigen-binding fragment thereof is administered every other week (q2w); or
(f) the second antibody or antigen-binding fragment thereof is administered subcutaneously; or
(g) the second antibody or antigen-binding fragment thereof is administered subcutaneously using an autoinjector, a needle and syringe, or a pen delivery device.

26-31. (canceled)

32. The method of claim 20, wherein:
(a) the first antibody or antigen binding fragment thereof is administered subcutaneously at a dose of about 0.1 mg to about 600 mg, about 100 mg to about 400 mg, or about 300 mg; or
(b) the first antibody or antigen binding fragment thereof is administered subcutaneously at an initial dose of about 600 mg or about 300 mg; or
(c) the first antibody or antigen binding fragment thereof is administered subcutaneously in one or more secondary doses of about 300 mg; or
(d) the first antibody or antigen-binding fragment thereof is administered intravenously at a dose of 10 mg/kg.

33-36. (canceled)

37. The method of claim 1,wherein:
(a) the allergic asthma is mild allergic asthma, optionally wherein the allergic asthma is mild persistent allergic asthma; or
(b) the subject is allergic to house dust mite (HDM) allergen; or
(c) the subject is a non-smoker; or
(d) the subject is clinically stable and requires short-acting inhaled β2 agonist (SABA) use on a per needed basis to control asthma symptoms; or
(e) loss of asthma control (LOAC) is reduced in the subject; or
(f) an asthma symptom selected from the group consisting of cough, wheezing, and short-acting inhaled β2 agonist use is reduced in the subject.

38-43. (canceled)

44. The method of claim 1, wherein one or more asthma-associated parameter(s) are improved in the subject.

45. The method of claim 44, wherein the asthma-associated parameter is selected from the group consisting of forced expiratory volume in 1 second (FEV1), peak expiratory flow (PEF), forced vital capacity (FVC), forced expiratory flow (FEF) 25%-75%, and reduction of the frequency or the dosage of short-acting inhaled β2 agonist use in the subject; or
wherein pre-bronchodilator FEV1 is improved in the subject.

46. (canceled)

47. The method of claim 1, wherein:
(a) blood eosinophil levels are reduced in the subject; or
(b) one or both of asthma control questionnaire 5-question version (ACQ-5) score and asthma quality of life questionnaire with standardized activities (AQLQ) score are improved in the subject; or
(c) the frequency or the dosage of SABA use in the subject is reduced in the subject; or
(d) bronchial allergen challenge (BAC)-induced lung inflammation is reduced in the subject; or
(e) a type 2 cytokine level is decreased in the subject, optionally wherein the type 2 cytokine is one or both of IL-13 and IL-5; or
(f) a cytokine level or a chemokine level is decreased in the subject, wherein the cytokine or the chemokine is selected from the group consisting of tumor necrosis factor-alpha (TNFα), thymus and activation-regulated chemokine (TARC),pulmonary and activation-regulated chemokine (PARC), CCL1, CCL26, FCER2, SIGLEC8, CCL17, and eotaxin-3; or
(g) early allergen response (EAR) or late allergen response (LAR) is reduced in the subject; or
(h) FEV1 is improved in the subject by at least 20%, 30%, 40%, 50%, 60%, or 70%; or
(i) FeNO levels are reduced in the subject; or
(j) serum levels of sST2, IL-33, calcitonin, or matrix metalloproteinase-12 (MMP12) are reduced in the subject; or
(k) serum levels of CCL26, CCL17, or SIGLEC8 are reduced in the subject; or
(l) serum levels of ASAP1-IT1, AX747757, BC042385, PABPC1P2, AB209315, AX748268, TCEAL5, CCL13, CLC, CACNG8, GPR82, GATA1, PRSS33, FFAR3, LGALS12, ASB2, PTGDR2, IL-13, IL-5, PTGDS, or RD3 are reduced in the subject.

48-59. (canceled)

60. A method for reducing a cytokine level or a chemokine level in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for reducing expression of one or more allergic asthma signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for reducing expression of any combination of type 2 inflammatory cytokine and type 2 chemokine signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for reducing expression of one or more eosinophil signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for reducing expression of one or more type 2 inflammatory signature genes in a subject having allergic asthma, comprising administering to the subject an antibody or antigen-binding fragment thereof that specifically binds interleukin-33 (IL-33) and comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16; or
a method for reducing a cytokine level or a chemokine level in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26; or
a method for reducing expression of one or more allergic asthma signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26; or
a method for reducing expression of any combination of type 2 inflammatory cytokine and type 2 chemokine signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26; or
a method for reducing expression of one or more eosinophil signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26; or
a method for reducing expression of one or more type 2 inflammatory signature genes in a subject having allergic asthma, comprising administering to the subject an antibody or antigen-binding fragment thereof that specifically binds interleukin-4 receptor (IL-4R) and comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26.

61. The method of claim 60, wherein the cytokine is one or both of IL-13 and IL-5; or
the cytokine or chemokine is selected from the group consisting of TNFα, TARC, PARC, CCL1, CCL26, FCER2, SIGLEC8, CCL17 and eotaxin-3.

62. (canceled)

63. The method of claim 60, wherein serum levels of sST2, IL-33, calcitonin or MMP12 are reduced in the subject; or
serum levels of CCL26, CCL17 or SIGLEC8 are reduced in the subject.

64. (canceled)

65. (canceled)

66. The method of claim 60, wherein the one or more allergic asthma signature genes are selected from the group consisting of BC042385, AB209315, LOC100607117, BC035084, LOC145474, AX747853, TIMP1, NT5DC2, LOC541471, AREG, PTPN7, RUNDC3, XXYLT1, FAM159A, PTGDS, TESC, ITGB2-AS1, D0574721, CLDN9, LOC100132052, AGAP7, NBEAL2, NTNG2, FLJ45445, KCNH3, POU51P3, OUG1, KIF21B, HSPA7, GAPT, BX6485Q2, PRR52, PIK3R6, LTC4S, CLEC11A, TRABD2A, DLGAP3, VDR, DKFZp686M11215, SIGLEC12, BC016361, BC052769, and RHOH; or
the one or more allergic asthma signature genes are selected from the group consisting of ASAP1-IT1, AX747757, BC042385, PABPC1P2, AB209315, AX748268, TCEAL5, CCL17, CCL13, CCL26, CLC, CACNG8, GPR82, GATA1, PRSS33, FFAR3, LGALS12, ASB2, PTGDR2, SIGLEC8, IL13, IL5, PTGDS and RD3.

67. (canceled)

68. (canceled)

69. The method of claim 60, wherein the type 2 inflammatory cytokine and chemokine signature genes are selected from the group consisting of IL-5, CCL1, IL-13, GATA2, CCL26, FCER2, CACNG8, CLC, GATA1, LGALS12, SIGLEC8, GGT5, CCL17 and MMP10.

70. (canceled)

71. (canceled)

72. The method of claim 60, wherein the one or more one or more eosinophil signature genes are selected from the group consisting of II1RL1, ADARB1, SIGLEC8, ASB2, VSTM1, SYNE1, CLC, PTPN7 and HDC.

73. (canceled)

74. The method of claim 60, wherein the one or more type 2 inflammatory signature genes are selected from the group consisting of IL-4, IL-13, CCL26, CCL13, CCL17. CCL11, POSTN, IL-5 and IL-9.

75. The method of claim 60,wherein:
the anti-IL-33 antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10; or
the anti-IL-33 antibody or antigen-binding fragment thereof comprises a heavy chain comprising the amino acid sequence of SEQ ID NO; 18 and a light chain comprising the amino acid sequence of SEQ ID NO: 20; or
the anti-IL4R antibody or antigen binding fragment thereof comprises a heavy chain variable region (HCVR) comprising the amino acid sequence of SEQ ID NO: 27 and a light chain variable region (LCVR) comprising the amino acid sequence of SEQ ID NO: 28; or
the anti-IL4R antibody or antigen-binding fragment thereof comprises dupilumab.

76-93. (canceled)

94. The method of claim 60, further comprising administering to the subject an antibody or antigen-binding fragment thereof that specifically binds IL-4R, wherein the antibody or antigen-binding fragment thereof comprises three heavy chain complementarity determining region (HCDR) sequences comprising SEQ ID NOs: 21, 22 and 23, and three light chain complementarity determining region (LCDR) sequences comprising SEQ ID NOs: 24, 25 and 26; or
further comprising administering to the subject an antibody or antigen-binding fragment thereof that specifically binds IL-33, wherein the antibody or antigen-binding fragment thereof comprises three heavy chain complementarity determining region (HCDR) sequences comprising SEQ ID NOs: 4, 6 and 8, and three light chain complementarity determining region (LCDR) sequences comprising SEQ ID NOs: 12, 14 and 16.

95. (canceled)
